(19) United States
(12) Patent Application Publication
(10) Pub. No.: US 2022/0339277 A1
(43) Pub. Date: Oct. 27, 2022

(54) IMMUNOGENIC COMPOSITIONS

(71) Applicant: GLAXOSMITHKLINE BIOLOGICALS SA, Rixensart (BE)

(72) Inventors: Nathalie Isabelle DEVOS, Rixensart (BE); Steven Clement SIJMONS, Rixensart (BE)

(73) Assignee: GLAXOSMITHKLINE BIOLOGICALS SA, Rixensart (BE)

(21) Appl. No.: 17/763,354

(22) PCT Filed: Sep. 30, 2020
(86) PCT No.: PCT/EP2020/077416

§ 371 Date: Mar. 24, 2022

(30) Foreign Application Priority Data
Oct. 1, 2019 (EP) 19200878.7

(51) Int. Cl. A61K 39/02; A61P 31/04

(52) U.S. Cl. CPC A61K 39/099; A61P 31/04; A61K 2039/552

(57) ABSTRACT

The invention relates to bacterial strains, particularly for use in the field of vaccines, in particular to the field of the prevention or treatment of infections caused by bacterium of the Bordetella genus.

### TECHNICAL FIELD

The present invention relates to the field of vaccines, in particular to the prevention or treatment of infections caused by bacteria of the Bordetella genus, particularly Bordetella pertussis.

### BACKGROUND TO THE INVENTION

Pertussis, caused by Bordetella pertussis, is a highly contagious airway infection. Acute infection can cause severe illness characterized by respiratory failure, pulmonary hypertension, leucocytosis, and death. Pertussis is a vaccine-preventable disease, with either acellular pertussis (aP) or whole-cell pertussis (wP) vaccines being used worldwide. However, the disease has persisted in vaccinated populations, and epidemiological data has reported a worldwide increase in pertussis incidence in recent years.

Several hypotheses have been postulated, including the evolution of pertussis strains and the decreased duration of protection from aP vaccines compared to wP vaccines. One of the fields of research aimed at controlling the re-emergence of pertussis is directed towards the development of new vaccines.

Vesicles derived from pathogens have been used in the development of immunogenic compositions such as vaccines [1]. The use of outer membrane vesicles (OMVs) or their derivatives could potentially deliver a broad spectrum of antigens in their native form.

OMVs comprise the bacterial lipopolysaccharide (LPS), which is composed of a highly variable O antigen, a less variable core oligosaccharide and a highly conserved lipid moiety designated lipid A. Bacterial lipooligosaccharides (LOS) share similar lipid A structures with an identical array of functional activities as LPSs, but they lack O-antigen units. In the art, the term “LPS” is often used to refer to both LPS and LOS bacterial saccharides.

However, the presence of LPS/LOS in OMVs may present issues from a clinical and regulatory point of view. For example, when LPS/LOS is degraded in the human body, the production of excessive proinflammatory cytokines can be elicited. Through their interaction with human TLR4 they can potentially cause a number of adverse effects (reactogenicity) such as fever, chills, shock, and a variety of other symptoms depending on the particular organism and the condition of the patient.

The endotoxic activity of the LPS/LOS is mainly determined by the composition of its lipid A moiety, which consists of a glucosamine disaccharide substituted with one or two phosphate groups and a variable number of acyl chains [2].

WO2018/167061 [3] describes the use of LpxA variants derived from either Pseudomonas aeruginosa (LpxA_{Pa}) or Neisseria meningitidis (LpxA_{Nm}) to reduce the length of the C3′ acyl chain in Bordetella pertussis. The exogenous LpxA genes were expressed from a plasmid, whilst the genomic, endogenous LpxA gene was knocked out. Episomal expression of LpxA_{Pa} had the effect of reducing the length of some acyl chains and the authors noted that TLR4 stimulation was reduced. However, the strains exhibited strong growth defects. In addition, expression of LpxA_{Nm} was lethal. Similar strong growth defects were observed when an LpxD variant derived from Pseudomonas aeruginosa was episomally expressed in B. pertussis. Whilst the results confirm that reactogenicity can be modified by engineering of lipid A, the growth defect of the strains is a major impediment to the use of such strains in vaccine manufacturing.

Thus, there remains a need for improvements suitable for the manufacture of immunogenic compositions such as vaccines.

### SUMMARY OF THE INVENTION

The Applicant has developed novel recombinant Bordetella pertussis bacterial strains that overcome the issues observed in the art and are particularly suitable for the preparation of whole-cell antigens and/or outer membrane vesicle components. These components may be used in immunogenic compositions such as vaccines.

In a First Aspect, the invention relates to a recombinant Bordetella pertussis bacterium. In particular, the invention relates to a recombinant Bordetella pertussis bacterium which comprises: at least one genomic LpxA gene encoding an LpxA protein, wherein the LpxA protein comprises a mutation at position 170 and/or a mutation at position 229 relative to SEQ ID NO: 1; and/or at least one genomic insertion of a heterologous LpxD gene.

In this First Aspect, the invention preferably relates to a recombinant Bordetella pertussis bacterium which comprises at least one genomic LpxA gene encoding an LpxA protein, wherein the LpxA protein comprises a mutation at position 170 and/or a mutation at position 229 relative to SEQ ID NO: 1.

The amino acid sequence of the wild-type LpxA protein from Bordetella pertussis (LpxA_{Bpe}) is provided as SEQ ID NO: 1. The amino acid sequence of the LpxA protein from Bordetella parapertussis (LpxA_{Bpa}) is provided as SEQ ID NO: 2.

The invention may relate to a recombinant Bordetella pertussis bacterium which comprises at least one genomic LpxA gene encoding an LpxA protein, wherein the LpxA gene is derived from Bordetella parapertussis (LpxA_{Bpa}).

In such an embodiment, the invention relates to a recombinant Bordetella pertussis bacterium which comprises at least one genomic LpxA gene encoding an LpxA protein, wherein the LpxA protein has SEQ ID NO: 2.

The term “mutation” as used herein, refers to deletion, addition, or substitution of amino acid residues in the amino acid sequence of a protein or polypeptide as compared to the amino acid sequence of a reference protein or polypeptide, particularly a wild-type protein or polypeptide.

The term “substitution” when referring to an amino acid sequence, refers to a change in an amino acid for a different amino acid or amino-acid moiety. For example, when referring to LpxA, a substitution refers to a change in an amino acid relative to SEQ ID NO: 1. Substitution of an amino acid at one particular location in the protein sequence is referred to using the following annotation “(amino acid residue in wild type protein)(amino acid position)(amino acid residue in mutated protein)”. For example, G229A refers to a substitution of a Glycine (G) residue at the 229th position of the amino acid sequence of the reference protein (here SEQ ID NO:1) by an Alanine (A) residue (in the mutant).

Particularly, the mutation at position 170 is a substitution relative to SEQ ID NO:1. Yet more particularly, the mutation at position 170 is a substitution of Glycine. Still yet more particularly, the mutation at position 170 is a substitution of Glycine with Serine (G170S).

Particularly, the mutation at position 229 is a substitution relative to SEQ ID NO:1. Yet more particularly, the mutation at position 229 is a substitution of Glycine. Still yet more particularly, the mutation at position 229 is a substitution of Glycine with Alanine (G229A).

Particularly the LpxA protein comprises a mutation at position 170 and a mutation at position 229 relative to SEQ ID NO:1. Yet more particularly, the mutation at position 170 is a substitution of Glycine and the mutation at position 229 is a substitution of Glycine. Still yet more particularly, the mutation at position 170 is a substitution of Glycine with Serine (G170S) and the mutation at position 229 is a substitution of Glycine with Alanine (G229A). Still yet even more particularly, the LpxA protein comprises a Serine residue at position 170, and an Alanine residue at position 229. Particularly, and in addition to the mutations at positions 170 and/or 229, the LpxA protein has at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity to either SEQ ID NO: 1 or SEQ ID NO: 2. In some embodiments, the LpxA protein has 90% sequence identity to SEQ ID NO: 2. In some embodiments, the LpxA protein has the amino acid sequence of SEQ ID NO: 2 (i.e. it has 100% sequence identity).

The amino acid sequence of an LpxD protein from Comamonas testosteroni (LpxD_{Ct}) is provided as SEQ ID NO: 3. The amino acid sequence of an LpxD protein from P. aeruginosa (LpxD_{Pa}) is provided as SEQ ID NO: 4.

Particularly, the heterologous LpxD gene encodes an LpxD protein having at least 85%, at least 86%, at least 87%, at least 88%, at least 89%, at least 90%, at least 91%, at least 92%, at least 93%, at least 94%, at least 95%, at least 96%, at least 97%, at least 98% or at least 99% amino acid sequence identity with either SEQ ID NO:3 or SEQ ID NO: 4. More particularly, the heterologous LpxD gene encodes an LpxD protein having at least 95% amino acid sequence identity with either SEQ ID NO:3 or SEQ ID NO: 4. Yet more particularly, the heterologous LpxD gene encodes an LpxD protein having 100% sequence identity with an amino acid sequence selected from the group consisting of SEQ ID NO:3 or SEQ ID NO: 4. In some embodiments, the heterologous LpxD gene encodes an LpxD protein having the amino acid sequence of SEQ ID NO:3. In some embodiments, the heterologous LpxD gene encodes an LpxD protein having the amino acid sequence of SEQ ID NO: 4.

The term “heterologous LpxD gene” refers to an LpxD gene or nucleotide sequence from a different species than the species of the host organism it is introduced into, in the context of the present invention, the heterologous LpxD gene or nucleotide sequence is not normally found in wild-type strains of Bordetella pertussis. In certain embodiments, the nucleotide sequence of the heterologous LpxD gene may be codon optimised for expression in Bordetella pertussis.

The term “genomic” refers to the bacterial cell genome, i.e. chromosomal DNA, whilst “genomic insertion” is used to refer to stable integration into the bacterial cell genome thereby excluding transient or episomal expression, such as expression of genes in a plasmid.

In the First Aspect of the invention, the expression or activity of the endogenous LpxA gene in the recombinant Bordetella pertussis bacterium is modified, reduced, suppressed or inactivated relative to a wild-type Bordetella pertussis bacterium. In some embodiments of the invention, expression or activity of the endogenous LpxD gene in the recombinant Bordetella pertussis bacterium may be modified, reduced, suppressed or inactivated relative to expression or activity seen in a wild-type Bordetella pertussis bacterium. In some embodiments of the invention, expression or activity of both the endogenous LpxA gene and endogenous LpxD gene in the recombinant Bordetella pertussis bacterium is modified, reduced, suppressed or inactivated relative to a wild-type Bordetella pertussis bacterium.

In some embodiments, the expression or activity of the endogenous LpxA gene is reduced, supressed or inactivated by knocking out the endogenous LpxA gene. In some embodiments, the expression or activity of the endogenous LpxA gene is modified, reduced, suppressed or inactivated by replacing the endogenous LpxA gene via, for example, a knock-in. In some embodiments, the expression or activity of the endogenous LpxA gene is modified by mutating the endogenous LpxA gene, particularly by mutation at positions 170 and/or 229. In some embodiments, the expression or activity of the endogenous LpxD gene is reduced, supressed or inactivated by knocking out the endogenous LpxD gene. In some embodiments, the expression or activity of the endogenous LpxD gene is modified, reduced, suppressed or inactivated by replacing the endogenous LpxD gene via, for example, a knock-in. By way of non-limiting example, a knock-out may be achieved by removing, for example, deleting, part or all of the endogenous gene or by inactivating the endogenous promoter which activates expression of the respective endogenous gene.

Surprisingly, recombinant Bordetella pertussis bacterial strains of the invention do not exhibit growth abnormalities observed in the art.

Particularly, the growth profile of a population of recombinant Bordetella pertussis bacteria of the invention is comparable to that of a population of wild type, parental strain of Bordetella pertussis bacteria. More particularly, the growth curve of a population of the recombinant Bordetella pertussis bacteria is comparable to that of a population of wild type, parental strain of Bordetella pertussis bacteria. For example, the values of the elapsed time and growth rate in standard fermentation processes of the population of recombinant Bordetella pertussis bacteria and a population of the wild type gram-negative bacteria are comparable. More particularly, the values of elapsed time and growth rate for the population of recombinant Bordetella pertussis bacteria and the population of wild type Bordetella pertussis bacteria vary by less than 20%, for example, less than 15%, less than 10% or less than 5%. Yet more particularly, the values of elapsed time and growth rate for the population of recombinant Bordetella pertussis bacteria and a population of wild-type Bordetella pertussis bacteria are substantially similar.

The recombinant Bordetella pertussis bacterial strains of the present invention express Lipid A that exhibits reduced endotoxic activity when compared to Lipid A expressed by the wild-type Bordetella pertussis bacterium, particularly when measured using TLR4 stimulation assays, particularly human TLR4 stimulation assays, for example, through NF-kB induction of a recombinant HEK-TLR4 reporter cell line. One skilled in the art will be aware of other suitable assays.

Particularly, recombinant Bordetella pertussis bacterial strains according to the present invention are derived from the Tohama I parental strain, and more particularly a Tohama I PTg strain, a strain of Tohama I that expresses genetically detoxified pertussis toxoid. As used herein, the term “parental strain” takes its general meaning in the art to refer to the strain of Bordetella pertussis used as the starting microorganism to construct the recombinant strain. The parental strain is generally the wild-type or comparator strain against which characteristics of the recombinant strains of the invention are compared. Recombinant Bordetella pertussis bacteria of the invention will generally be isogenic with its parent strain, except for the genetic modification or modifications of the invention described herein.

In a Second Aspect of the invention, there is provided an outer membrane vesicle (OMV), particularly a population of outer membrane vesicles, derived from the recombinant Bordetella pertussis bacterium of the First Aspect. There is also provided a whole-cell antigen derived from the recombinant Bordetella pertussis bacterium of the First Aspect.

In a Third Aspect of the invention, there is provided an immunogenic composition comprising at least one OMV or a population of OMVs according to the Second Aspect and a pharmaceutically acceptable excipient. Particularly, immunogenic compositions may also comprise an adjuvant. In some embodiments, immunogenic compositions of the invention may comprise at least one antigen in addition to those present in the OMV or OMVs. In preferred embodiments of the invention, the at least one additional antigen is selected from the group consisting of (1) pertussis toxoid (PT), (2) FHA, (3) pertactin (PRN), (4) FIM2/FIM3, (5) adenylate cyclase, (6) diphtheria toxoid (DT), (7) tetanus toxoid (TT), (8) inactivated polio virus (IPV), (9) hepatitis B surface antigen and (10) Hib PRP.

In a Fourth Aspect of the invention, there is provided the recombinant Bordetella pertussis bacterium according to the First Aspect, at least one OMV or population of OMVs of the Second Aspect or the immunogenic composition of the Third Aspect for use in inducing an immune response in a suitable mammal.

Particularly, there is provided the recombinant Bordetella pertussis bacterium according to the First Aspect, at least one OMV or population of OMVs of the Second Aspect or the immunogenic composition of the Third Aspect for use in therapy and/or prophylaxis, for example, for use as a vaccine.

Particularly, there is provided a method for inducing an immune response in a suitable mammal comprising administering to the suitable mammal the recombinant Bordetella pertussis bacterium according to the First Aspect, at least one OMV or population of OMVs of the Second Aspect or the immunogenic composition of the Third Aspect.

Particularly, there is provided the recombinant Bordetella pertussis bacterium according to the First Aspect, at least one OMV or population of OMVs of the Second Aspect or the immunogenic composition of the Third Aspect for use in the manufacture of a medicament, for example, for therapy and/or prophylaxis, for example, for use as a vaccine. In one embodiment, there is provided the recombinant Bordetella pertussis bacterium according to the First Aspect, at least one OMV or population of OMVs of the Second Aspect or the immunogenic composition of the Third Aspect for use in the manufacture of a medicament for the treatment and/or prophylaxis of disease caused by Bordetella pertussis.

In a Fifth Aspect of the invention, there is provided a method of modulating the reactogenicity of the Lipid A of a Bordetella pertussis bacterium, comprising stably integrating into the genome of the bacterium at least one gene selected from the group consisting of LpxA and LpxD, wherein:

(i) the LpxA gene encodes an LpxA protein having at least 80% sequence identity with SEQ ID NO: 1 or 2 and wherein the protein comprises Serine at position 170 (S170) and/or Alanine at position 229 (A229) when numbered in accordance with SEQ ID NO: 1 or 2; and/or

(ii) the LpxD gene encodes an LpxD protein having at least 90% amino acid sequence identity with SEQ ID NO:3 or SEQ ID NO: 4.

Preferably, in this Fifth Aspect of the invention, the method of modulating the reactogenicity of the Lipid A of a Bordetella pertussis bacterium, comprises stably integrating into the genome of the bacterium an LpxA gene, wherein the LpxA gene encodes an LpxA protein having at least 80% sequence identity with SEQ ID NO: 1 or 2 and wherein the protein comprises Serine at position 170 (S170) and/or Alanine at position 229 (A229) when numbered in accordance with SEQ ID NO: 1 or 2.

In one embodiment, the method comprises stably integrating into the genome of the bacterium an LpxA gene is derived from Bordetella parapertussis (LpxA_{Bpa}).

### BRIEF DESCRIPTION OF THE DRAWINGS

FIG. 1(A). Structure of lipid A from Bordetella pertussis; and FIG. 1(B). Structure of lipid A from Bordetella parapertussis, where the dotted lines show naturally occurring variant structures, in particular, one and two secondary C16 acyl chains that may additionally be present, and a minority variant with loss of the C10 chain.

FIG. 2. Alignment of the LpxA amino acid sequences from the Bordetella genus.

FIG. 3. Fermentation profile of B. pertussis Tomaha I PTg. Curve 1: pH regulated at 7.2 by addition of acetic acid (Curve 3) to avoid the basification of cell broth resulting in stopping the culture; Curve 2: dissolved oxygen concentration; Curve 4: stirring speed manipulated to keep dissolved oxygen concentration (DO) at 25% to avoid oxygen limitation (DO/speed regulation); Curve 5: temperature regulated at 35° C.

FIG. 4. Fermentation profile of a recombinant B. pertussis strain of the invention expressing LpxA from B. parapertussis (ALpxA_{Bpe}/LpxA_{Bpa}). Curve 2: stirring speed manipulated to keep dissolved oxygen concentration (DO) at 25% to avoid oxygen limitation (DO/speed regulation); Curve 3: temperature regulated at 35° C.; Curve 4: pH regulated at 7.2 by addition of acetic acid (Curve 1) to avoid the basification of cell broth resulting in stopping the culture; Curve 5: dissolved oxygen concentration. NB: Due to clogging of the exhaust filter, the DO and stirring speed dropped to 0 during the filter replacement process for about 20 min. This did not affect the results of the fermentation.

FIG. 5. Stirring speed fermentation profiles of ALpxA_{Bpe}/LpxA_{Bpa} (dashed) and the parental strain, Tohama I PTg (black).

FIG. 6(A). Structural analysis of LOS by LC-MS—OMVs from B. pertussis of ALpxA_{Bpe}/LpxA_{Bpa}, expressing genomically integrated LpxA_{Bpa}. FIG. 6(B). The LOS structure as determined by LC-MS. The solid lines show the structure of this LOS. The dotted lines illustrate the difference between this LOS structure and the LOS structure found in wild type B. pertussis strain Tohama 1.

FIG. 7(A). Structural analysis of LOS by LC-MS—OMVs from B. pertussis, episomally expressing LpxA_{Bpa}. FIG. 7(B). A minority variant LOS structure as determined by LC-MS (where the dotted lines illustrate the difference between this LOS and the LOS structure found in wild type B. pertussis strain Tohama I).

FIG. 8(A). Structural analysis of LOS by LC-MS—OMVs from B. pertussis ΔArnT/ΔLpxA_{Bpe}/LpxA_{Bpa}, an ArnT and LpxA_{Bpe} double knock-out variant expressing genomically integrated LpxA_{Bpa}. FIG. 8(B). The LOS structure as determined by LC-MS. The solid lines show the structure of this LOS. The dotted lines illustrate the difference between this LOS structure and the LOS structure found in wild type B. pertussis strain Tohama 1.

FIG. 9(A). Structural analysis of LOS by LC-MS—OMVs from B. pertussis ΔLpxA_{Bpe}/LpxA_{Bpa}/ADNT/LpxD_{Pa}, a LpxA_{Bpe} and DNT double knock-out variant expressing genomically integrated LpxA_{Bpa} and LpxD_{Pa}. FIG. 9(B). An LOS structure as determined by LC-MS. The solid lines show the structure of this LOS. The dotted lines illustrate the difference between this LOS structure and the LOS structure found in wild type B. pertussis strain Tohama 1.

FIG. 10(B). Structural analysis of LOS by LC-MS—OMVs from B. pertussis ΔLpxA_{Bpe}/LpxA_{Bpa}/ADNT/LpxD_{Pa}/ΔLpxD_{Bpe}, a LpxA_{Bpe}, DNT and LpxD_{Bpe} triple knock-out variant expressing genomically integrated LpxA_{Bpa} and LpxD_{Pa}. FIG. 10(B). The LOS structure as determined by LC-MS. The solid lines show the structure of this LOS. The dotted lines illustrate the difference between this LOS structure and the LOS structure found in wild type B. pertussis strain Tohama 1.

FIG. 11(A). Structural analysis of LOS by LC-MS—OMVs from B. pertussis LpxD_{Pa}, episomally expressing LpxD_{Pa}. FIG. 11(B). An LOS structure as determined by LC-MS. The solid lines show the structure of this LOS. The dotted lines illustrate the difference between this LOS structure and the LOS structure found in wild type B. pertussis strain Tohama 1.

FIG. 12(A). Structural analysis of LOS by LC-MS—OMVs from B. pertussis ΔArnT/PagL_{Bbr}, an ArnT knock-out variant expressing PagL_{Bbr}. FIG. 12(B). The LOS structure as determined by LC-MS. The solid lines show the structure of this LOS. The dotted lines illustrate the difference between this LOS structure and the LOS structure found in wild type B. pertussis strain Tohama 1.

FIG. 13(A). Structural analysis of LOS by LC-MS—OMVs from wild type B. pertussis Tomaha I PTg. FIG. 13(B). The LOS structure as determined by LC-MS.

FIG. 14. In vitro activation of TLR4 signalling in HEK-hTLR4 cells. HEK-TLR4 cells were stimulated based on protein content with OMV from recombinant B. pertussis strains; OMV from B. pertussis Tohama I PTg strain (wild type control); or based on equivalent fraction of human dose (vaccine controls) with BEXSERO, QUINVAXEM or INFANRIX HEXA vaccines. Non-stimulated cells were included as a negative control (data not shown).

FIG. 15 In vitro activation of TLR4 signalling in HEK-hTLR4 cells. HEK-TLR4 cells were stimulated based on LOS content with OMV from recombinant B. pertussis strains; OMV from B. pertussis Tohama I PTg strain (wild type control); purified LPS from B. pertussis Tomaha I PTg; or a detoxified purified LPS from Neisseria meningitis B DMsbB strain (MsbB). Non-stimulated cells were included as a negative control.

FIG. 16. In vitro activation of TLR4 signalling in HEK-hTLR4 cells. HEK-TLR4 cells were stimulated based on protein content with OMV from recombinant B. pertussis strains; OMV from B. pertussis Tohama I PTg strain (wild type control); OR BEXSERO, QUINVAXEM and INFANRIX HEXA vaccines. Non-stimulated cells were included as a negative control.

FIG. 17. In vitro activation of TLR4 signalling in HEK-hTLR4 cells. HEK-TLR4 cells were stimulated based on LOS content with OMV from recombinant B. pertussis strains; OMV from B. pertussis Tohama I PTg strain (wild type control); purified LPS from B. pertussis; or OMV from a detoxified Neisseria meningitis B □3MsbB strain (□MsbB). Stimulations were normalized by LOS content. Non-stimulated cells were included as a negative control.

FIG. 18(A). Structural analysis of LOS by LC-MS—OMVs from B. pertussis, episomally expressing LpxE_{Fn}. FIG. 18(B). A variant of the LOS structure as determined by LC-MS, where the variant structure has a phosphate removed (position indicated by the black arrow).

FIG. 19(A). Structural analysis of LOS by LC-MS—OMVs from B. pertussis, episomally expressing LpxE_{Rc}. FIG. 19(B). A variant of the LOS structure as determined by LC-MS, where the variant structure has a phosphate removed (position indicated by the black arrow).

FIG. 20. Structural analysis of LOS by LC-MS—OMVs from B. pertussis, episomally expressing LpxF_{Fn}. FIG. 20(B). The LOS structure as determined by LC-MS showing no identifiable impact on LOS structure.

FIG. 21 in vitro activation of TLR4 signalling in HEK-hTLR4 cells. HEK-TLR4 cells were stimulated based on LOS content with OMV from recombinant B. pertussis strains expressing LpxE_{Fn}. OMV from B. pertussis Tohama I PTg strain (WT PTg) were used as a positive control, non-stimulated cells were included as a negative control.

FIG. 22. In vitro activation of TLR4 signalling in HEK-hTLR4 cells. HEK-TLR4 cells were stimulated based on protein content with OMV from recombinant B. pertussis strains expressing LpxE_{Fn}. OMV from B. pertussis Tohama I PTg strain (WT PTg) were used as a positive control, non-stimulated cells were included as a negative control.

FIG. 23. Individual antibody titers (anti-PRN IgG) and geometric mean with 95% confidence interval measured by ELISA from the study conducted in the nasopharynx colonisation model in Balb/c mice.

FIG. 24. Individual values and geometric means of the bacterial load as defined by the mean of number of colony-forming unit (CFU) per nasal cavity, shown by group and by day, from the study conducted in the nasopharynx colonisation model in Balb/c mice.

FIG. 25. Individual anti-PRN IgG titers measured by ELISA at day 28 and geometric mean titres (with error bars that represent the associated 95% confidence intervals) from the study conducted in the lung clearance model in Balb/c mice.

FIG. 26. Individual colony-forming unit (CFU) counts (small dots) and geometric means (large connected dots) by group as function of day from the study conducted in the lung clearance model in Balb/c mice.

FIG. 27(A) to (F). Six cytokines (i.e.: IL1a, IL6, TNFa, IL1b, IL10, MIP1a) known to be associated with reactogenicity mechanisms measured in culture supernatants for the different serial dilutions.

### DETAILED DESCRIPTION OF THE INVENTION

The invention relates to recombinant Bordetella pertussis strains comprising LPS that exhibits reduced endotoxicity when compared to LPS of wild type Bordetella pertussis bacterium. References in this application to LPS encompass LOS as well. The endotoxicity of both LPS and LOS is mainly determined by the composition of the lipid A moiety

Surprisingly, the present inventors have discovered that the bacterium of the invention also exhibits a bacterial growth profile comparable to that of wild-type, non-recombinant, Bordetella pertussis bacterium.

Use of the term “wild-type” in relation to a bacterium refers to the typical strain as it occurs in nature. Such wild-type strains lack the modifications of the invention described herein. Thus, the term “wild type” is used to refer to microorganisms of the same strain as the microorganism of the invention but which lack the genetic modifications of the invention, for example the LpxA and/or LpxD genetic modifications described herein. The wild-type strain may also be referred to as the parental strain. As used herein, “endogenous” gene or protein is used to refer to the unmodified gene (nucleic acid sequence) or protein (amino acid sequence) found in the wild-type, parental, strain of Bordetella pertussis bacterium from which the recombinant bacterium was originally derived.

On the other hand, the term “exogenous” is used to refer to genes (nucleic acid sequences) or proteins (amino acid sequences) that are not naturally found in the wild-type Bordetella pertussis bacterial strain from which the recombinant bacterium is derived. The term “exogenous” may be used interchangeably with the term “heterologous”.

The present invention relates to recombinant Bordetella pertussis strains which comprise: at least one genomic LpxA gene encoding an LpxA protein, wherein the LpxA protein comprises a mutation at position 170 and/or a mutation at position 229 relative to SEQ ID NO: 1; and/or at least one genomic insertion of a heterologous LpxD gene.

The Bacterium

The recombinant Bordetella pertussis bacteria of the invention can be derived from various parental strains of Bordetella pertussis including, by way of non-limiting example: Tohama I, for example Bordetella pertussis Moreno-Lopez (ATCC: BAA-589), BP165, BPW28, 10536, 18323, 1135, BAA-2706, BAA-2705 or PT/28G [W28] (ATCC: 53894). Preferably, the recombinant Bordetella pertussis bacterium of the present invention is derived from a strain that expresses a genetically detoxified pertussis toxoid, particularly the genetically detoxified pertussis toxoid referred to as PT-9K/129G. Recombinant Bordetella pertussis strains expressing the PT-9K/129G genetically detoxified pertussis toxoid comprise two amino acid substitutions within the S1 subunit of PT, specifically R9K and E129G (see for example EP0396964). Particularly, the strain of Bordetella pertussis from which the recombinant bacterium is derived is Tohama I. More particularly the recombinant Bordetella pertussis bacterium is derived from a strain of Tohama I that expresses a genetically detoxified pertussis toxoid. Yet more particularly, the recombinant Bordetella pertussis bacterium is derived from a strain of Tohama I that expresses the genetically detoxified pertussis toxoid PT-9K/129G. Still yet more particularly, the recombinant Bordetella pertussis bacterium comprises an S1 gene that has been modified to include the mutations R9K and E129G and expresses the genetically detoxified pertussis toxoid PT-9K/129G.

Whilst pertussis toxin is secreted by Bordetella pertussis, it may still be present in isolated whole-cells and outer membrane vesicles. Therefore, the use of strains that express genetically detoxified PT is advantageous. Preferably, the recombinant Bordetella pertussis bacterium of the invention does not express the dermonecrotic toxin (DNT) gene.

Bacterial Lipopolysaccharide and Lipid A

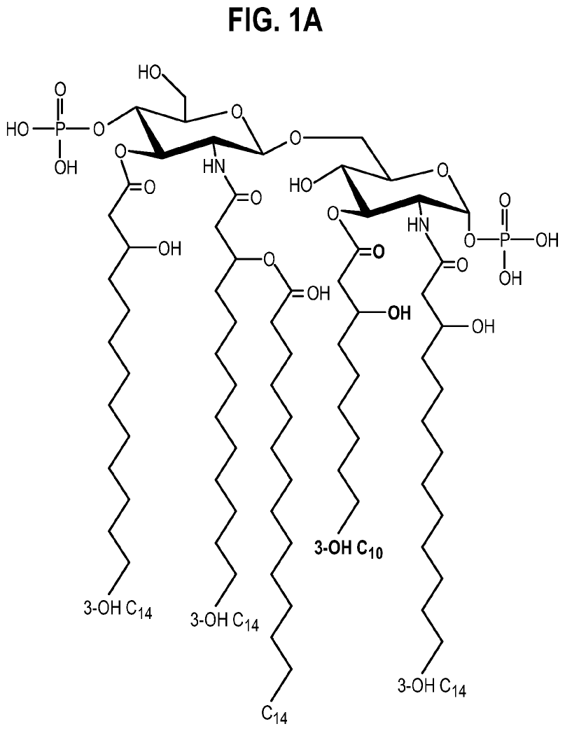

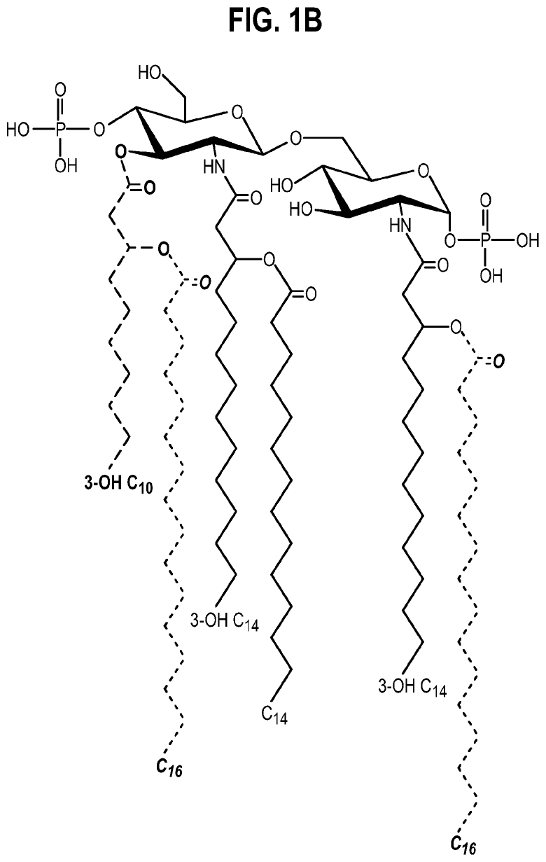

Bacterial lipopolysaccharide (LPS) is composed of a highly variable O antigen, a less variable core oligosaccharide and a highly conserved lipid moiety designated lipid A. Lipid A consists of a glucosamine disaccharide substituted with one or two phosphate groups and a number of acyl chains of varying length (FIG. 1). The endotoxic activity of the LPS is mainly determined by the composition of the lipid A moiety.

FIG. 1(A) shows the structure of lipid A from wild-type Bordetella pertussis. The present invention is based, in part, on the modification of the length of the C2, C2′ and/or C3′ acyl chains of lipid A in LPS of Bordetella pertussis. In a preferred embodiment, the C3′ acyl chains of lipid A in LPS of Bordetella pertussis are modified. In such an embodiment, the length of the C2 and C2′ acyl chains of lipid A in LPS of Bordetella pertussis may be modified as well. To this end, the lipid A biosynthetic pathway of Bordetella pertussis has been engineered.

LpxA Acyltransferases

Acyltransferase LpxA (referred to as ‘LpxA’) catalyses the transfer of ester-linked primary p-hydroxy-myristate to a monosaccharide precursor (UDP-glucosamine) at lipid A 3 and 3′ positions, determining the length of the acyl chains incorporated at both positions, which varies among gram-negative bacteria [4, 5, 6].

The endotoxic activity of the LPS is mainly determined by the composition of its lipid A moiety. Whilst variation in the number of acyl chains in lipid A can impact signalling through TLR4, the mechanism by which changes in these chains differentially affects TLR4 signalling has not been fully elucidated [2].

Thus, expression of heterologous LpxA genes might reduce the endotoxicity of LPS LpxA from Pseudomonas aeruginosa (LpxA_{Pa}) and Neisseria meningitidis (LpxA_{Nm}) catalyse the transfer of acyl chains with a length of 10 (C₁₀) or 12 (C₁₂) carbon atoms, respectively. However, in previous studies, such as those described in WO2018/167061, LpxA_{Pa} or LpxA_{Nm} genes were expressed episomally in B. pertussis, and the endogenous LpxA gene was inactivated. Episomal expression of LpxA_{Pa}, reduced the length of the C3′ acyl chain from C₁₄ to C₁₀ but the resulting recombinant strains exhibited strong growth defects making them unsuitable for large-scale fermentation and use. On the other hand, episomal expression of LpxA_{Nm} in Bordetella pertussis was lethal.

Surprisingly, the Inventors have found that, in Bordetella pertussis, genomic expression of LpxA from B. parapertussis (LpxA_{Bpa}) overcomes the fitness/growth issues observed in the art whilst also reducing endotoxicity of LPS.

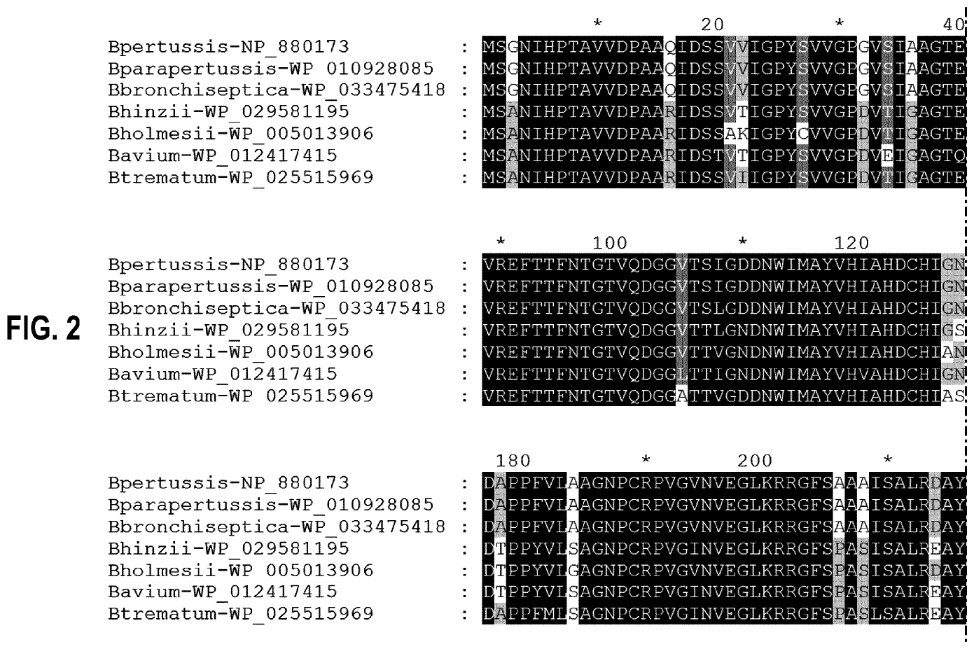

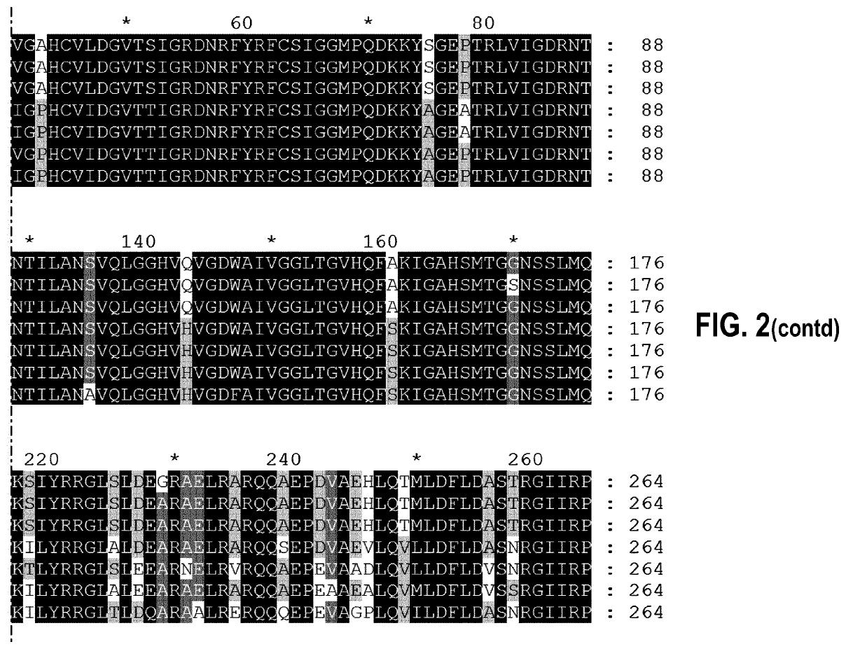

The amino acid sequence of LpxA from Bordetella parapertussis is provided as SEQ ID NO: 2. The amino acid sequence of LpxA from Bordetella pertussis is provided as SEQ ID NO: 1. The amino acid sequence of LpxA from B. pertussis differs from that of B. parapertussis in two residues, corresponding to amino acids at positions 170 and 229 of SEQ ID NOs: 1 and 2 (FIG. 2). The presence of particular substitutions at amino acid position 170 and/or position 229 is believed to alter the specificity of LpxA to selectively incorporate C₁₀ acyl chains at position C3′ of Lipid A. Thus, recombinant Bordetella pertussis strains of the invention may express an LpxA gene which encodes an LpxA amino acid sequence in which one or both of the amino acids at positions 170 and 229 (when numbered by reference to SEQ ID NO:1) have been substituted by an amino acid residue selected from the group consisting of Alanine (A), Serine (S), Threonine (T) and Cysteine (C). Particularly, the recombinant Bordetella pertussis strain may express an LpxA gene that encodes an LpxA amino acid sequence in which G170 has been substituted by Serine (G170S) and/or in which G229 has been substituted by Alanine (G229A). Particularly, the recombinant Bordetella pertussis strain may express an LpxA gene that encodes an LpxA amino acid sequence that comprises a Serine residue at position 170 and/or an Alanine residue at position 229 (when numbered by reference to SEQ ID NO:1).

Amino acid substitutions or mutations may be introduced by standard molecular biology techniques such as random mutagenesis, site-directed mutagenesis, directed evolution, gene replacement and the like.

Preferably, the LpxA gene, in addition to the mutation(s) described above, encodes an amino acid sequence having at least 80% sequence identity with SEQ ID NO: 2, for example, 81%, at least 82%, at least 83%, at least 84%, at least 8%, at least 86%, at least 87%, at least 88%, at least 89% or 90%, at least 91%, at least 92%, at least 93%, at least 94%, at least 95%, at least 96%, at least 97%, at least 98%, at least 99% or 100% sequence identity.

Particularly, sequence identity of two sequences may be determined using the GENEPAST algorithm provided by GenomeQuest, Inc. The algorithm was formerly called “Kerr” and performs an arithmetical determination of percent sequence identity without any biological weighing (Dufresne et al, Nature Biotechnology 20, 1269-1271 (2002), or Andree et al, World Patent Information, 2008, vol. 30, issue 4, pages 300-308). Generally, sequence identity is calculated with respect to and along the entire (i.e. full) length of the longest of the sequences being compared.

LpxD Acyltransferase

LpxD acyltransferase (LpxD) catalyses the transfer of amide-linked p-hydroxy-myristate at the 2 and 2′ positions of lipid A, determining the length of the acyl chains incorporated at both positions, which again varies among gram-negative bacteria [6].

The expression of exogenous LpxD variants, may improve the endotoxic profile of the bacterial LPS. Thus, recombinant Bordetella pertussis strains of the invention may comprise at least one heterologous LpxD gene. Particularly, the heterologous LpxD gene is integrated into the bacterial genome.

A suitable amino acid sequence of LpxD from C. testosteroni (LpxD_{Ct}) is provided as SEQ ID NO: 3. A suitable amino acid sequence of LpxD from P. aeruginosa (LpxD_{Pa}) is provided as SEQ ID NO: 4.

Thus, in some embodiments the at least one heterologous LpxD gene is an LpxD gene from P. aeruginosa. In some embodiments the at least one heterologous LpxD gene is an LpxD gene from C. testosteroni. In some embodiments, the heterologous LpxD gene replaces the locus of the dermonecrotic toxin. In some embodiments, the heterologous LpxD gene replaces the endogenous LpxD gene. In some embodiments, the heterologous LpxD gene is under the control of the BP0840 promoter (outer membrane porin promoter). Even more particularly, the wild-type copy of the LpxD gene (LpxD_{Bp}) is knocked-out.

In some embodiments the amino acid sequence encoded by the at least one heterologous LpxD gene has at least 90% identity with SEQ ID NO:3.

Thus, in some embodiments, the amino acid sequence encoded by the at least one LpxD gene has at least 90%, at least 95% or 100% identity with SEQ ID NO:3. More particularly, the LpxD amino acid sequence has at least 90%, at least 91%, at least 92%, at least 93%, at least 94%, at least 95%, at least 96%, at least 97%, at least 98%, at least 99% or 100% identity with SEQ ID NO:3.

In some embodiments the amino acid sequence encoded by the at least one heterologous LpxD gene has at least 90% identity with SEQ ID NO:4.

Thus, in some embodiments, the amino acid sequence encoded by the at least one LpxD gene has at least 90%, at least 95% or 100% identity with SEQ ID NO:4. More particularly, the LpxD amino acid sequence has at least 90%, at least 91%, at least 92%, at least 93%, at least 94%, at least 95%, at least 96%, at least 97%, at least 98%, at least 99% or 100% identity with SEQ ID NO:4.

Particularly, the recombinant Bordetella pertussis bacterium of the invention expresses both a heterologous LpxD gene and an LpxA gene that encodes an LpxA amino acid sequence comprising a Serine residue at position 170 and an Alanine residue at position 229. More particularly, the B. pertussis bacterium expresses LpxA_{Bpa} and LpxD_{Pa}.

In some embodiments, the at least one LpxA gene and the at least one LpxD genes are integrated into the bacterial genome. In other embodiments, the LpxA gene is integrated into the bacterial genome whereas the LpxD gene may be expressed episomally.

The endogenous LpxA and/or LpxD genes of the recombinant bacterium may be inactivated by means well known to the person skilled in the art. For example, the genes may be knocked out.

Generally, the endogenous LpxA gene is mutated but it may be inactivated or replaced, for example with SEQ ID NO: 2. Generally, the endogenous LpxD gene is inactivated. In some embodiments, both the endogenous LpxA and endogenous LpxD genes are inactivated.

In some embodiments, the recombinant Bordetella pertussis bacterium of the invention comprises additional genetic modifications, such as expression of other exogenous genes and/or inactivation of other endogenous genes. By way of non-limiting example, (1) the endogenous ArnT gene (SEQ ID NO: 18) may be inactivated, for example, deleted or knocked-out; (2) the endogenous dermonecrotic toxin (DNT) gene (SEQ ID NO: 19) is inactivated, for example, deleted or knocked-out; (3) the bacterium may express PagL (SEQ ID NO: 16) from Bordetella bronchiseptica; (4) the bacterium may express an LpxE protein from a heterologous gram-negative bacterium (SEQ ID NO: 6 or 7); and/or (5) the bacterium may express an LpxF protein from a heterologous gram-negative bacterium (SEQ ID NO: 8).

LpxE and LpxF Phosphate Phosphatases

LpxE phosphate phosphatase (LpxE) and LpxF phosphate phosphatase (LpxF) are hydrolases that may act to remove a phosphate group from a lipid comprising a phosphate group. If one phosphate group is removed, the lipid A is “monophosphoryl lipid A”. With regard to Lipid A, the activity of LpxE and LpxF can result in the removal of phosphate groups at C1 and C4′ of the core disaccharide respectively.

LpxE phosphatases from Rhizobium leguminosarum (LpxE_{R}I) and Francisella novicida (LpxE_{Fn}) are disclosed in references [65] and [66]. LpxF phosphate phosphatase from Francisella novicida (LpxF_{Fn}) is disclosed in reference [67]. The authors further disclose heterologous expression of LpxE_{Rl}, LpxE_{Fn} and LpxF_{Fn} in E. coli [68] and [69]. However, these references do not disclose the use of LpxE or LpxF in Bordetella pertussis.

The present Inventors have now tested expression of exogenous LpxE and LpxF in Bordetella pertussis and demonstrated that the use of heterologous LpxE and/or LpxF alters the structure of Lipid A and may also change characteristics of lipid A, for example the endotoxic profile of the B. pertussis LPS.

Thus, recombinant Bordetella pertussis strains of the invention may comprise at least one heterologous LpxE gene and/or at least one heterologous LpxF gene. Particularly, the at least one heterologous LpxE gene and/or the at least one heterologous LpxF gene is/are integrated into the bacterial genome.

The LpxE is a phosphate phosphatase that specifically dephosphorylates the C1 position of the core disaccharide. Preferably, the LpxE is a heterologous LpxE gene from a bacterium selected from the group consisting of Rhizobium leguminosarum (LpxE_{Rl}) and Francisella novicida (LpxE_{Fn}). Suitable amino acid sequences of LpxE from Rhizobium leguminosarum and Francisella novicida are provided as SEQ ID NO: 6 and SEQ ID NO: 7 respectively.

In some embodiments, the heterologous LpxE gene replaces the locus of the ArnT gene (ΔArnT) encoding a glycosyltransferase. In some embodiments, the heterologous LpxE gene is under the control of the BP0840 promoter (outer membrane porin promoter). In some embodiments the amino acid sequence encoded by the at least one heterologous LpxE gene has at least 90% identity, at least 95% or 100% identity with SEQ ID NO:6 or SEQ ID NO:7. In one particular embodiment, the heterologous LpxE gene is LpxE_{Fn} under the control of the BP0840 promoter (outer membrane porin promoter) inserted into the bacterial genome and replacing the locus of the ArnT gene (ΔArnT).

In one embodiment, the recombinant Bordetella pertussis bacterium of the invention expresses a heterologous LpxE gene and an LpxA gene that encodes an LpxA amino acid sequence comprising a Serine residue at position 170 and an Alanine residue at position 229. More particularly, the B. pertussis bacterium expresses LpxABpa and an LpxE gene selected from the group consisting of LpxE_{Rl} and LpxE_{Fn}. In some embodiments, the LpxA and LpxE genes are integrated into the bacterial genome. In other embodiments, the LpxA gene is integrated into the bacterial genome but the LpxE gene is expressed episomally.

The LpxF is a phosphate phosphatase that specifically dephosphorylates the C4′ position of the core disaccharide. Preferably, the LpxF is a heterologous LpxF gene from Francisella novicida (LpxF_{Fn}). A suitable amino acid sequences of LpxF from Francisella novicida is provided as SEQ ID NO: 8.

In some embodiments, the at least one heterologous LpxF gene is an LpxF gene from Francisella novicida. In some embodiments, the heterologous LpxF gene replaces the locus of the ArnT gene encoding a glycosyltransferase. In some embodiments, the heterologous LpxF gene is under the control of the BP0840 promoter (outer membrane porin promoter). In some embodiments the amino acid sequence encoded by the at least one heterologous LpxF gene has at least 90% identity, at least 95% or 100% identity with SEQ ID NO:8.

Lipid a Acyl Chains

Generally, Lipid A of gram-negative bacteria consists of a β-(1→6)-linked disaccharide acylated with up to eight fatty acids of different lengths and complexities as well as charged substituents such as phosphate groups, phosphoethanolamine residues, or positively charged sugar residues and there are many variations within lipid A of different bacteria species. The acylation at positions 2 and 2′ of the disaccharide (reducing and terminal sugar residue, respectively) leads to amide-linked lipids, which is also the case for the 3- and 3′-positions when the 3-amino-3-deoxy-d-glucosamine is acylated. The hydroxyl groups at positions 3 and 3′ as well as the 3-hydroxyl group of the fatty acyl chains are also acylated, resulting in ester-linked substituents. The extent of acylation with 4-8 acyl groups attached to the β-(1→6)-linked disaccharide varies between species and more than a single form is often present in the lipid A from a certain bacterial species.

Generally, the length of the acyl chains at positions C2, C2′ and C3′ of Lipid A in wild-type Bordetella pertussis is C₁₄. As used herein, ‘C’ followed by an integer in subscript denotes a molecule based upon a carbon chain of a length denoted by the integer. Thus, C₁₄ and C₁₀ would denote molecules having a 14-carbon atom chain and a 10-carbon atom chain respectively.

The recombinant bacterium of the invention produces lipid A in which the length of the acyl chain at position C2 and/or C2′ and/or C3′ is reduced in comparison to the length of the corresponding acyl chains of lipid A produced by the wild-type gram-negative bacterium.

In a preferred embodiment of the recombinant bacterium of the invention, the bacterium produces lipid A in which the length of the acyl chain at position C3′ is reduced in comparison to the length of the corresponding acyl chains of lipid A produced by the wild-type gram-negative bacterium. In some embodiments, the bacterium produces a lipid A in which the length of the acyl chain at position C2 and/or C2′ is also reduced in comparison to the length of the corresponding acyl chains of lipid A produced by the wild-type gram-negative bacterium.

In a preferred embodiment, the length of the acyl chain at position C3′ is less than C₁₄, such as C₆ to C₁₂, C₈ to C₁₂ or C₁₀ to C₁₂, particularly C₁₀ or C₁₂, for example, C₆, C₈, C₁₀ or C₁₂. In a particularly preferred embodiment, the length of the acyl chain at position C3′ is Cao.

Particularly, the length of the acyl chain at position C2 is less than C₁₄, such as C₆ to C₁₂, C₈ to C₁₂ or C₁₀ to C₁₂, particularly C₁₀ or C₁₂, for example, C₆, C, C₁₀ or C₁₂. Particularly, the length of the acyl chain at position C2′ is less than C₁₄, such as C₆ to C₁₂, C to C₁₂ or C₁₀ to C₁₂, particularly C₁₀ or C₁₂, for example, C₆, C, C₁₀ or C₁₂. Particularly, the length of the acyl chain at position C3′ is less than C₁₄, such as C₆ to C₁₂, C₈ to C₁₂ or C₁₀ to C₁₂, particularly Cao or C₁₂, for example, C₆, C8, C₁₀ or C12.

In some embodiments, the length of the acyl chains at both position C2 and position C2′ are less than C₁₄, such as C₆ to C₁₂, C₈ to C₁₂ or C₁₀ to C₁₂, particularly C₁₀ or C₁₂, for example, C₆, C8, C₁₀ or C₁₂. In some embodiments, the length of the acyl chains at both position C2 and position C3′ are less than C₁₄, such as C₆ to C₁₂, C₈ to C₁₂ or C₁₀ to C₁₂, particularly C₁₀ or C₁₂, for example, C₆, C₈, C₁₀ or C₁₂. In some embodiments, the length of the acyl chains at both position C2′ and position C3′ are less than C₁₄, such as C₆ to C₁₂, C₈ to C₁₂ or C₁₀ to C₁₂, particularly C₁₀ or C₁₂, for example, C₆, C₈, C₁₀ or C₁₂. In some embodiments, the length of the acyl chains at position C2, position C2′ and position C3′ are less than C₁₄, such as C₆ to C₁₂, C₈ to C₁₂ or C₁₀ to C₁₂, particularly C₁₀ or C₁₂, for example, C₆, C8, C₁₀ or C₁₂.

Lipid A produced by the recombinant bacterium of the invention may exist as a mixture of species having acyl chains of differing lengths.

Thus, considering the total population of lipid A in the cell, at least 10% of the acyl chains at the C2 position are C₁₀ or C₁₂ and/or at least 10% of the acyl chains at the C2′ position are Cao or C₁₂ and/or at least 10% of the acyl chains at the C3′ position are C₁₀ or C₁₂. Particularly, at least 10% may be C₁₀ or C₁₂, at least 12%, at least 15%, at least 20%, at least 24%, at least 25%, at least 30%, at least 35%, at least 40%, at least 50%, at least 60%, at least 70%, at least 80%, at least 85%, at least 86%, at least 87%, at least 88%, at least 89%, at least 90%, at least 95%, at least 96%, at least 97%, at least 98%, at least 99% or 100% may be C₁₀ or C₁₂. Preferably at least 10% is C₁₀ or C₁₂, at least 80%, more preferably at least 90% is C₁₀ or C₁₂. Yet more particularly about 100% is C₁₀ or C₁₂.

Bacterial Growth

Recombinant strains of the invention are stable whilst also being suitable for growth at large scale, for example, in manufacturing. Surprisingly, the growth profile of recombinant Bordetella pertussis bacteria of the invention is comparable to that of the wild-type, parental, strain indicating that neither the LpxA mutation(s) nor the LpxD mutation(s) exemplified are detrimental to culture growth.

The recombinant bacterial strains of the invention are thus particularly suitable, for example, for the preparation of whole-cell bacterial antigens and/or OMVs, which may then be used as components of immunogenic compositions such as vaccines.

Thus, the growth rate of a culture of the recombinant gram-negative bacterium of the invention is comparable to that of a culture of the wild type, parental, strain. Particularly, in standard fermentation processes, for example in 10 litre fermentation volumes, the values of the elapsed time and growth rate are substantially similar, for example, vary by less than 20%, less than 15%, less than 10% or less than 5%.

Outer Membrane Vesicles (OMVs)

In a second aspect, the present invention relates to an outer membrane vesicle or population of outer membrane vesicles derived from the recombinant Bordetella pertussis bacterium of the first aspect.

OMVs are lipid bilayer nanoscale spherical particles (10-500 nm in diameter) naturally and constitutively released by Gram negative bacteria during growth. OMVs are generated through a “budding out” of the bacterial outer membrane and, consistent with this, they have a composition similar to that of the bacterial outer membrane, including lipopolysaccharide (LPS), glycerophospholipids, outer membrane proteins, and periplasmic components. Typically, outer membrane vesicles are prepared artificially from bacteria, and may be prepared using detergent treatment (e.g. with deoxycholate), or by non-detergent means.

Techniques for forming OMVs include treating bacteria with a bile acid salt detergent (e.g. salts of lithocholic acid, chenodeoxycholic acid, ursodeoxycholic acid, deoxycholic acid, cholic acid, ursocholic acid, etc.) or with sodium deoxycholate [7 and 8]. Other techniques may be performed substantially in the absence of detergent [9 and 10] using techniques such as sonication, homogenisation, microfluidisation, cavitation, osmotic shock, grinding, French press, blending, etc. OMVs used with the invention may be prepared using an OMV extraction buffer having about 0.5% deoxycholate or lower, for example, about 0.4%, about 0.3%, about 0.2%, about 0.1%, <0.05% or even zero.

OMVs may also form spontaneously during bacterial growth and are released into culture medium. Thus, OMVs can be obtained by culturing bacteria of the invention in culture medium, separating whole cells from the smaller OMVs in the culture medium. Bacterial vesicles can conveniently be separated from whole bacteria by filtration e.g. through a 0.22 μm filter. Bacterial filtrates may be clarified by centrifugation, for example high speed centrifugation (e.g. 20,000×g for about 2 hours). Another useful process for OMV preparation is described in [11] and involves ultrafiltration of crude OMVs, instead of high speed centrifugation. The process may involve a step of ultracentrifugation after the ultrafiltration takes place. A simple process for purifying bacterial vesicles is described in [12], comprising: (i) a first filtration step in which the vesicles are separated from the bacteria based on their different sizes, with the vesicles passing into the filtrate e.g. using a 0.22 μm microfiltration; and (ii) a second filtration step in which the vesicles are retained in the retentate e.g. using a 0.1 μm microfiltration. The two steps can both use tangential flow filtration.

OMVs of the invention will include LOS (also known as LPS). The pyrogenic effect of LOS in OMVs may be lower than that seen with the same amount of purified LOS. Therefore, preferably when looking at pyrogenicity, generally OMVs from bacteria of the invention will be compared with OMVs derived from the parental strain. LOS levels are expressed in International Units (IU) of endotoxin and can be tested by the LAL assay (limulus amebocyte lysate). LOS may be present at less than 2000 IU per μg of OMV protein.

In some embodiments of the invention, the OMV is a detergent extracted OMV (dOMV). The term “dOMV” encompasses a variety of proteoliposomic vesicles obtained by disruption of the outer membrane of a Gram-negative bacterium typically by a detergent extraction process to form vesicles therefrom. Preferably, the OMV is a detergent extracted OMV (dOMV). Particularly, OMVs of the invention are dOMVs prepared using deoxycholate. More particularly about 0.5% deoxycholate or lower, for example, about 0.4%, about 0.3%, about 0.2%, about 0.1% or about 0.05%.

dOMVs of the invention may have a size distribution of between from about 20 to about 500 nm as measured by Dynamic Light Scattering DLS technique. Particularly, dOMVs of the invention may have a size distribution of between from about 50 to about 500 nm. More particularly, the dOMVs may have a size distribution of between from about 75 to about 250 nm, for example, from about 75 to about 150 nm. Even more particularly, the dOMVs of the invention may have an average diameter of about 20, 25, 30, 35, 40, 45, 50, 55, 60, 65, 70, 75, 80, 85, 90, 95, 100, 105, 110, 115, 120, 125, 130, 135, 140, 145 or 150 nm.

In some embodiments, bacteria of the invention can be modified to have up-regulated antigens or expression of heterologous antigens (i.e. antigens not native to the particular bacterial strain). As a result of this modification, vesicles prepared from such modified bacteria contain higher levels of the up-regulated or heterologous antigen(s). The increase in expression in the vesicles (measured relative to a corresponding wild-type strain) of the up-regulated antigen is usefully at least 10%, measured in mass of the relevant antigen per unit mass of vesicle, and is more usefully at least 20%, 30%, 40%, 50%, 75%, 100% or more.

Whole-Cell Pertussis Antigens

Recombinant Bordetella pertussis bacteria of the invention may be used as whole-cell pertussis (wP) antigens, in the form of inactivated B. pertussis cells. Preparation of cellular pertussis antigens is well documented in the art and may be obtained by heat inactivation of phase I culture of B. pertussis cells. Quantities of wP antigens can be expressed in international units (IU). For example, the NIBSC supplies the ‘International Standard For Pertussis Vaccine’ (NIBSC code: 94/532), which contains 40 IU per ampoule.

When used in immunogenic compositions of the invention, the cellular pertussis antigen is typically present in an amount that is capable of eliciting an immune response when administered. Ideally, the cellular pertussis antigen can elicit a protective immune response. The amount of wP antigen in immunogenic compositions of the invention is typically at least 4 IU/dose. The wP antigen may be adsorbed onto or mixed with an aluminium adjuvant, for example, an aluminium phosphate adjuvant.

TLR4 Signalling Cascade

The bacterium of the invention produces lipid A with reduced endotoxic activity when compared to that of lipid A from the wild-type strain, particularly when measured using a TLR4 stimulation assay.

TLR4 stimulation assays are well known to the skilled person. The stimulation of the TLR4 signalling cascade can lead to the activation of NF-κB and/or activator protein 1 (AP-1) transcription factors that are contributing to the expression of several pro-inflammatory cytokines, e.g. IL-1, IL-6, IL-8 and TNF-α and thus play a key role in inflammation. Determination of transcription activity in certain lines, for example HEK-Blue™-hTLR4 cells (Invivogen), can be used to assess stimulation of the TLR4 signalling cascade. More specifically, the inducible reporter gene secreting embryonic alkaline phosphatase (SEAP) of the HEK-Blue™-hTLR4 cells is under control of the IL-12 p40 minimal promoter fused to five NF-κB and AP-1 binding sites. Upon stimulation with a TLR4 ligand, activation of NF-kB and AP-1 induces the secretion of alkaline phosphatase that can be quantified in the supernatant by measuring optical density at 655 nm (OD₆₅₅).

Endotoxic activity of bacteria of the invention, for example whole-cell antigens or OMVs derived from bacteria of the invention may be determined using a TLR4 stimulation assay, particularly a human TLR4 stimulation assay, more particularly a human TLR4 stimulation assay with optical density (OD) measured at 655 nm. Particularly, the endotoxic activity of whole-cells or OMVs of the invention is compared to that of the wild-type parental strain. The capacity to activate the TLR4 signalling by OMVs derived from the bacterium of the invention may be compared to that of OMVs derived from wild type strains as well as to those from other bacteria or commercial immunogenic compositions, for example, BEXSERO (Meningitis B vaccine containing OMVs from Neisseria) or QUINVAXEM (whole-cell (wP) pertussis vaccine).

More particularly, endotoxic activity of OMVs of the invention exhibit a reduced or decreased activation of the TLR4 signalling cascade compared to the endotoxic activity of OMVs derived from the wild-type bacterium. Specifically, the OD₆₅₅ values obtained using the TLR4 stimulation assay when testing the LpxA and/or LpxD mutants should be similar, for example about ±0.5 OD₆₅₅, to the OD₆₅₅ values obtained using the non-stimulated cells and/or to the OD₆₅₅ values observed with cells stimulated with an acellular pertussis (aP) vaccine.

In some embodiments, the TLR4 stimulation assays show a partial decrease in activation of the TLR4 signalling cascade for the OMVs of the invention in comparison to OMVs derived from wild-type bacteria. Specifically, the OD₆₅₅ values observed with the LpxA and/or LpxD bacteria or OMVs of the invention may be higher (≥0.5 OD₆₅₅) than the OD₆₅₅ values observed in the non-stimulated cells and/or to the OD₆₅₅ values observed with cells stimulated with aP vaccine, but lower than the OD₆₅₅ values observed with the cells stimulated with OMV derived from wild type bacterium and/or with a wP vaccine and/or the OMV-containing MenB vaccine.

Immunogenic Compositions

In a third aspect, the invention relates to immunogenic compositions comprising at least one outer membrane vesicle derived from the recombinant Bordetella pertussis bacterium of the invention and a pharmaceutically acceptable excipient.

OMVs of the invention are useful as active ingredients in immunogenic compositions. Similarly, whole-cell antigens comprising whole recombinant Bordetella pertussis cells of the invention which have been killed and deactivated (e.g. by treatment with formalin and/or heat), are also useful as active ingredients in immunogenic compositions.

The term “immunogenic composition” broadly refers to any composition that may be administered to elicit an immune response, such as an antibody or cellular immune response, against an antigen or antigens present in the composition. Thus, compositions of the invention are immunogenic.

When the immunogenic compositions prevent, ameliorate, palliate or eliminate disease from the subject, then such compositions may be referred to as a vaccine. Vaccines according to the invention may either be prophylactic (i.e. to prevent infection) or therapeutic (i.e. to treat infection), but will typically be prophylactic. In certain embodiments, the immunogenic composition is a vaccine. Prophylactic vaccines do not guarantee complete protection from disease because even if the patient develops antibodies, there may be a lag or delay before the immune system is able to fight off the infection. Therefore, and for the avoidance of doubt, the term prophylactic vaccine may also refer to vaccines that ameliorate the effects of a future infection, for example by reducing the severity or duration of such an infection.

The terms “protection against infection” and/or “provide protective immunity” means that the immune system of a subject has been primed (e.g by vaccination) to trigger an immune response and repel infection. Particularly, the immune response triggered is capable of repelling infection against Bordetella pertussis. A vaccinated subject may thus get infected, but is better able to repel the infection than a control subject. Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. Commonly, the desired result is the production of an antigen (e.g., pathogen)-specific immune response that is capable of or contributes to protecting the subject against the pathogen. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

The term “antigen” refers to a substance that, when administered to a subject, elicits an immune response directed against the substance. In the context of the present invention, OMVs of the invention are antigens. Particularly, when administered to a subject the immunogenic composition will elicit an immune response directed against Bordetella, for example, Bordetella pertussis. Particularly the immune response directed against Bordetella is protective, that is, it can prevent or reduce infection or colonisation caused by Bordetella, particularly Bordetella pertussis. Compositions may thus be pharmaceutically acceptable. Generally, immunogenic compositions include components in addition to the antigens e.g. they typically include one or more pharmaceutical carrier(s) and/or excipient(s). A thorough discussion of such components is available in reference [13].

A “pharmaceutically acceptable carrier” is a carrier that does not itself induce the production of antibodies. Such carriers are well known to those of ordinary skill in the art and include, by way of non-limiting example, polysaccharides, polylactic acids, polyglycolic acids, amino acid copolymers, sucrose, trehalose, lactose, and lipid aggregates (such as oil droplets or liposomes). Immunogenic compositions may also contain diluents, such as water, saline, glycerol, and the like. Sterile pyrogen-free, phosphate-buffered physiologic saline is a typical diluent. Such compositions may also include, auxiliary substances, such as wetting or emulsifying agents, pH buffering substances, etc.

Compositions will generally be administered to a subject, for example a patient, particularly a suitable mammal, such as a human, in aqueous form. Prior to administration, however, the composition may have been in a non-aqueous form. For instance, although some vaccines are manufactured in aqueous form, then filled and distributed and administered also in aqueous form, other vaccines are lyophilised during manufacture and are reconstituted into an aqueous form at the time of use. Thus, a composition of the invention may be dried, such as a lyophilised formulation.

The composition may include a preservative such as thiomersal or 2-phenoxyethanol. It is preferred, however, that the vaccine should be substantially free from (i.e. less than 5 μg/ml) mercurial material e.g. thiomersal-free. Vaccines containing no mercury are more preferred. Thiomersal-free vaccines are particularly preferred.

To control tonicity, a physiological salt, such as a sodium salt may be included. Sodium chloride (NaCl) may be used, which may be present at between 1 and 20 mg/ml e.g. about 10±2 mg/ml NaCl.

Compositions will generally have an osmolality of between 200 mOsm/kg and 400 mOsm/kg, particularly between 240-360 mOsm/kg, and more particularly within the range of 290-310 mOsm/kg.

Compositions may include one or more buffers. Typical buffers include: a phosphate buffer; a Tris buffer; a borate buffer; a succinate buffer; a histidine buffer; or a citrate buffer. Buffers will typically be included in the 5-20 mM range.

The pH of a composition will generally be between 5.0 and 8.1, and more typically between 6.0 and 8.0 e.g. 6.5 and 7.5, or between 7.0 and 7.8.

The composition is preferably sterile. Particularly the composition is non-pyrogenic e.g. containing <1 EU (endotoxin unit, a standard measure) per dose, for example, <0.1 EU per dose. The composition may be gluten free.

The composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Immunogenic compositions, for example human vaccines, are typically administered in a dosage volume of about 0.5 ml, although a half dose (i.e. about 0.25 ml) may be administered, for example, to children.

Immunogenic compositions of the invention may also comprise one or more immunoregulatory agents. Preferably, one or more of the immunoregulatory agents include one or more adjuvants. As used herein, “adjuvant” means a compound or substance (or combination of compounds or substances) that, when administered to a subject in conjunction with an antigen or antigens, for example as part of an immunogenic composition or vaccine, increases or enhances the subject's immune response to the administered antigen or antigens (compared to the immune response obtained in the absence of adjuvant). The adjuvants may include a TH1 adjuvant and/or a TH2 adjuvant, further discussed below.

Adjuvants which may be used in compositions of the invention include, mineral containing compositions such as aluminium salts and calcium salts. The compositions of the invention may include mineral salts such as hydroxides (e.g. oxyhydroxides), phosphates (e.g. hydroxyphosphates, orthophosphates), sulphates, etc. [e.g. see chapters 8 & 9 of ref.14], or mixtures of different mineral compounds, with the compounds taking any suitable form (e.g. gel, crystalline, amorphous, etc.), and with adsorption being preferred. The mineral containing compositions may also be formulated as a particle of metal salt.

The adjuvants known as “aluminium hydroxide” are typically aluminium oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm⁻¹ and a strong shoulder at 3090-3100 cm⁻¹ [chapter 9 of ref. 14] The degree of crystallinity of an aluminium hydroxide adjuvant is reflected by the width of the diffraction band at half height (WHH), with poorly-crystalline particles showing greater line broadening due to smaller crystallite sizes. The surface area increases as WHH increases, and adjuvants with higher WHH values have been seen to have greater capacity for antigen adsorption. A fibrous morphology (e.g. as seen in transmission electron micrographs) is typical for aluminium hydroxide adjuvants. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

The adjuvants known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 1.2.

Hydroxyphosphates can be distinguished from strict AlPO₄ by the presence of hydroxyl groups. For example, an IR spectrum band at 3164 cm⁻¹ (e.g. when heated to 200° C.) indicates the presence of structural hydroxyls [chapter 9 of ref. 14].

The PO₄/Al³⁺ molar ratio of an aluminium phosphate adjuvant may be between 0.3 and 1.2, particularly between 0.8 and 1.2, and more particularly 0.95±0.1. The aluminium phosphate may be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate-like morphology as seen in transmission electron micrographs). Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention may have a PZC of between 4.0 and 7.0, more particularly between 5.0 and 6.5 e.g. about 5.7.

Suspensions of aluminium salts used to prepare compositions of the invention may contain a buffer (e.g. a phosphate or a histidine or a Tris buffer), but this is not always necessary. The suspensions are preferably sterile and pyrogen-free. A suspension may include free aqueous phosphate ions e.g. present at a concentration between 1.0 and 20 mM, between 5 and 15 mM, particularly about 10 mM. The suspensions may also comprise sodium chloride.

In some embodiments, an adjuvant component includes a mixture of both an aluminium hydroxide and an aluminium phosphate. In this case there may be more aluminium phosphate than hydroxide e.g. a weight ratio of at least 2:1 e.g. ≥5:1, ≥6:1, ≥7:1, ≥8:1, ≥9:1, etc. In some embodiments, an adjuvant component includes aluminium phosphate. In some embodiments, an adjuvant component includes aluminium hydroxyphosphate sulfate.

The concentration of Al⁺⁺⁺ in a composition for administration to a patient may be between from 10 mg/ml to 0.01 mg/ml, for example, less than 5 mg/ml, less than 4 mg/ml, less than 3 mg/ml, less than 2 mg/ml, less than 1 mg/ml, for example, about 5 mg/ml, about 4 mg/ml, about 3 mg/ml, about 2 mg/ml, about 1 mg/ml, about 0.3 mg/ml, about 0.05 mg/ml or about 0.01 mg/ml. A particular range is between from about 0.3 mg/ml to about 1 mg/ml. In some embodiments, a maximum of 0.85 mg/dose is preferred, for example about 0.5 mg/I or about 0.3 mg/ml.

Oil emulsion compositions suitable for use as adjuvants in the invention include squalene-water emulsions, such as MF59 [Chapter 10 of ref. 14; see also ref. 15] (5% Squalene, 0.5% Tween 80, and 0.5% Span 85, formulated into submicron particles using a microfluidizer). Complete Freund's adjuvant (CFA) and incomplete Freund's adjuvant (IFA) may also be used.

Saponin formulations may also be used as adjuvants in the invention. Saponins are a heterogeneous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™.

AS01 is an Adjuvant System containing MPL (3-O-desacyl-4′-monophosphoryl lipid A), QS21 ((Quillaja saponaria Molina, fraction 21) Antigenics, New York, N.Y., USA) and liposomes. AS01B is an Adjuvant System containing MPL, QS21 and liposomes (50 μg MPL and 50 μg QS21). AS01E is an Adjuvant System containing MPL, QS21 and liposomes (25 μg MPL and 25 μg QS21). In one embodiment, the immunogenic composition or vaccine comprises AS01. In another embodiment, the immunogenic composition or vaccine comprises AS01 B or AS01 E. In a particular embodiment, the immunogenic composition or vaccine comprises AS01E. AS02 is an Adjuvant system containing MPL and QS21 in an oil/water emulsion. AS02V is an Adjuvant System containing MPL and QS21 in an oil/water emulsion (50 μg MPL and 50 μg QS21). AS03 is an Adjuvant System containing α-Tocopherol and squalene in an oil/water (o/w) emulsion. AS03A is an Adjuvant System containing α-Tocopherol and squalene in an o/w emulsion (11.86 mg tocopherol). AS03B is an Adjuvant System containing α-Tocopherol and squalene in an o/w emulsion (5.93 mg tocopherol). AS03C is an Adjuvant System containing α-Tocopherol and squalene in an o/w emulsion (2.97 mg tocopherol). In one embodiment, the immunogenic composition or vaccine comprises AS03. AS04 is an Adjuvant System containing MPL (50 μg MPL) adsorbed on an aluminium salt (500 μg A13+). In one embodiment, the immunogenic composition or vaccine comprises AS04. A system involving the use of QS21 and 3D-MPL is disclosed in WO 94/00153. A composition wherein the QS21 is quenched with cholesterol is disclosed in WO 96/33739. An additional adjuvant formulation involving QS21, 3D-MPL and tocopherol in an oil in water emulsion is described in WO 95/17210. In one embodiment the immunogenic composition additionally comprises a saponin, which may be QS21. The formulation may also comprise an oil in water emulsion and tocopherol (WO 95/17210). Unmethylated CpG containing oligonucleotides (WO 96/02555) and other immunomodulatory oligonucleotides (WO 0226757 and WO 03507822) are also preferential inducers of a TH1 response and are suitable for use in the present invention.

Additional adjuvants include Toll like receptor agonists, (in particular Toll like receptor 2 agonist, Toll like receptor 3 agonist, Toll like receptor 4 agonist, Toll like receptor 7 agonist, Toll like receptor 8 agonist and Toll like receptor 9 agonist). Compositions of the invention may include one or more small molecule immunopotentiators. For example, the composition may include a TLR2 agonist (e.g. Pam3CSK4), a TLR4 agonist (e.g. an aminoalkyl glucosaminide phosphate, such as E6020), a TLR7 agonist (e.g. imiquimod), a TLR8 agonist (e.g. resiquimod (also a TLR7 agonist)) and/or a TLR9 agonist (e.g. IC31). Any such agonist ideally has a molecular weight of <2000 Da.

TLR7 agonists used with the invention ideally includes at least one adsorptive moiety. The inclusion of such moieties in TLR agonists allows them to adsorb to insoluble aluminium salts (e.g. by ligand exchange or any other suitable mechanism) and improves their immunological behaviour. Phosphorus-containing adsorptive moieties are particularly useful, and so an adsorptive moiety may comprise a phosphate, a phosphonate, a phosphinate, a phosphonite, a phosphinite, etc. The TLR agonist may include at least one phosphonate group. In particular embodiments, a composition of the invention may include a TLR7 agonist which includes a phosphonate group. This phosphonate group can allow adsorption of the agonist to an insoluble aluminium salt. In some embodiments, the TLR agonist is 3-(5-amino-2-(2-methyl-4-(2-(2-(2-phosphonoethoxy)ethoxy)ethoxy)phenethyl)benzo[f]-[1,7]naphthyridin-8-yl)propanoic acid, as shown below.

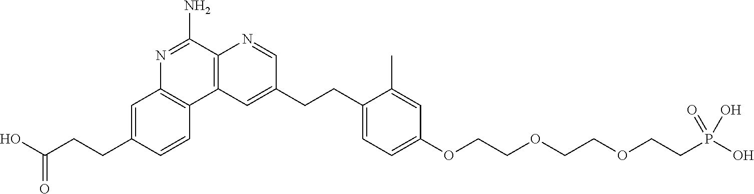 [SMILES: Cc1cc(OCCOCCOCCP(=O)(O)O)ccc1CCc1cnc2c(N)nc3cc(CCC(=O)O)ccc3c2c1]

Preferred TLR agonists are water-soluble. Thus, they can form a homogenous solution when mixed in an aqueous buffer with water at pH 7 at 25° C. and 1 atmosphere pressure to give a solution which has a concentration of at least 50 μg/ml. The term “water-soluble” thus excludes substances that are only sparingly soluble under these conditions.

Compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The composition may be prepared for topical administration e.g. as an ointment, cream or powder. The composition may be prepared for pulmonary administration e.g. as an inhaler, using a fine powder or a spray. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilised antigens.

Where a composition is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Combination Vaccines

Immunogenic compositions of the invention will generally be combination vaccines and, in addition to OMVs of the invention, may include further protective antigen(s) from Bordetella pertussis and at least one pathogen other than Bordetella pertussis.

The additional protective antigen(s) can be viral and/or bacterial. In addition to Bordetella pertussis, typical bacterial pathogens include, but are not limited to: Corynebacterium diphtheriae; Clostridium tetani; Haemophilus influenzae type b. Typical viral pathogens include, but are not limited to: poliovirus and hepatitis B virus. For the avoidance of doubt, reference to additional antigens is intended to refer to further antigenic components included in the immunogenic compositions of the invention beyond the constituents of the OMVs. For example, small amounts of pertussis toxoid may be found as a constituent of OMVs but reference to pertussis toxoid as a further antigen refers to an amount of pertussis toxoid that is specifically added over and above the OMV constituent, for example, as an isolated protein antigen.

Acellular Pertussis Antigens

In addition to OMVs of the invention, immunogenic compositions of the invention may further comprise one or more acellular pertussis (aP) antigens, particularly selected from the following well-known and well-characterized B. pertussis antigens: (1) detoxified pertussis toxin (pertussis toxoid, or ‘PT’); (2) filamentous hemagglutinin (‘FHA’); (3) pertactin (also known as ‘PRN’ or the ‘69 kiloDalton outer membrane protein’); (4) fimbriae type 2 (‘FIM2’); (5) fimbriae type 3 (‘FIM 3’). It is most preferred that both detoxified PT and FHA are used. In some embodiments PT, FHA and PRN are used. These antigens are preferably prepared by isolation from B. pertussis culture grown in modified Stainer-Scholte liquid medium. PT and FHA can be isolated from the fermentation broth (e.g. by adsorption on hydroxyapatite gel), whereas pertactin can be extracted from the cells by heat treatment and flocculation (e.g. using barium chloride). The antigens can be purified in successive chromatographic and/or precipitation steps. PT and FHA can be purified by hydrophobic chromatography, affinity chromatography and size exclusion chromatography. Pertactin can be purified by ion exchange chromatography, hydrophobic chromatography and size exclusion chromatography.

FHA and pertactin may be treated with formaldehyde prior to use according to the invention. PT may be detoxified by treatment with formaldehyde and/or glutaraldehyde. As an alternative to this chemical detoxification procedure preferably the PT may be a mutant PT in which enzymatic activity has been reduced by mutagenesis, for example, genetically detoxified PT, preferably the PT-9K/129G.

In some embodiments, immunogenic compositions of the invention further comprise the fimbrial antigens FIM2 and FIM3.

The aP antigen(s) may be used in an unadsorbed state, or may be adsorbed onto one or more aluminium salt adjuvant(s) before being used. Typically, the aP antigens are substantially free from mercurial preservatives such as thimerosal.

The acellular pertussis antigens may be present in the immunogenic compositions of the invention in an amount that is capable of eliciting an immune response when administered.

Ideally, the acellular pertussis antigens can elicit a protective immune response. Quantities of acellular pertussis antigens are typically expressed in micrograms. The concentration of PT in a vaccine is usually between 5 and 50 μg/ml. Typical PT concentrations are 5 μg/ml, 16 μg/ml, 20 μg/ml or 50 μg/ml, for example, about 20 μg per dose or about 25 μg per dose. The concentration of FHA in a vaccine is usually between 10 and 50 μg/ml. Typical FHA concentrations are 10 μg/ml, 16 μg/ml or 50 μg/ml, for example, about 20 μg per dose or about 25 μg per dose. The concentration of pertactin in a vaccine is usually between 5 and 16 μg/ml. Typical pertactin concentrations are 5 μg/ml, 6 μg/ml or 16 μg/ml, for example, about 3 μg per dose or about 8 μg per dose. FIM2 and FIM3 may be present at a concentration 5 and 16 μg/ml. Typical concentrations of FIM2 and FIM3 are 5 μg/ml, 10 μg/ml or 20 μg/ml, for example, for example, about 5 μg per dose or about 10 μg per dose. Booster vaccines for adolescents and adults typically contain 2.5 to 8 μg PT, between 4 and 8 μg FHA and between 2.5 and 8 μg pertactin per 0.5 ml dose. Typically, a booster vaccine comprises 4 μg PT, 4 μg FHA and 8 μg pertactin, more preferably 5 μg PT, 2.5 μg FHA and 2.5 μg pertactin, per 0.5 ml dose. A paediatric vaccine usually comprises 7 μg PT, 10 μg FHA and 10 μg pertactin, per 0.5 ml dose.

Where the aqueous component includes each of PT, FHA and pertactin, their weight ratios can vary, but may be e.g. about 16:16:5, about 5:10:6, about 20:20:3, about 25:25:8, or about 10:5:3 (PT:FHA:PRN).

Diphtheria

Corynebacterium diphtheriae causes diphtheria. Diphtheria toxin can be treated (e.g. using formalin or formaldehyde) to remove toxicity while retaining the ability to induce specific anti-toxin antibodies after injection. The diphtheria toxoids used in diphtheria vaccines are well known in the art. Preferred diphtheria toxoids are those prepared by formaldehyde treatment. The diphtheria toxoid can be obtained by growing C. diphtheriae in growth medium (e.g. Fenton medium, or Linggoud & Fenton medium), which may be supplemented with bovine extract, followed by formaldehyde treatment, ultrafiltration and precipitation. Particularly the growth medium for growing C. diphtheriae is free from animal-derived components. The toxoided material may then be treated by a process comprising sterile filtration and/or dialysis. Alternatively, genetically detoxified diphtheria toxin (e.g., CRM197) may be used which may be formaldehyde-treated to maintain long-term stability during storage.

The diphtheria toxoid may be adsorbed onto an adjuvant such as an aluminium salt adjuvant.

Quantities of diphtheria toxin and/or toxoid in a composition are generally measured in the ‘Lf’ unit (“flocculating units”, or the “limes flocculating dose”, or the “limit of flocculation”), defined as the amount of toxin/toxoid which, when mixed with one International Unit of antitoxin, produces an optimally flocculating mixture [16,17]. For example, the NIBSC supplies ‘Diphtheria Toxoid, Plain’ [18], which contains 300 LF per ampoule, and also supplies ‘The 2nd International Reference Reagent For Diphtheria Toxoid For Flocculation Test’ (NIBSC Code: 02/176) which contains 900 Lf per ampoule. The concentration of diphtheria toxin or toxoid in a composition can readily be determined using a flocculation assay by comparison with a reference material calibrated against such reference reagents.

The immunizing potency of diphtheria toxoid in a composition is generally expressed in international units (IU). The potency can be assessed by comparing the protection afforded by a composition in laboratory animals (typically guinea pigs) with a reference vaccine that has been calibrated in IUs. NIBSC supplies the ‘4th WHO International Standard for Diphtheria Toxoid (Adsorbed)’(NIBSC code: 07/216) which contains 213 IU per ampoule, and is suitable for calibrating such assays.

A three-dilution assay can be used to determine the potency of the compositions of the invention. After immunization, guinea-pigs are bled or challenged either by the subcutaneous or by the intradermal route. In an alternative embodiment, mice are used in place of guinea pigs. When guinea pigs or mice are bled, the antitoxin levels of the individual animals are titrated by means of toxin neutralization tests performed using in vivo or in vitro serological methods that have been validated on vaccines of the types being tested. In one embodiment, diphtheria toxoids produced in fermentation medium comprising animal-derived components are used for validation. The potency of the composition of the invention is calculated using appropriate statistical methods. For three-dilution assays, the limits of the 95% confidence intervals of the estimate of potency is within 50-200% of the estimated potency unless the lower limit of the 95% confidence interval of the estimated potency is greater than 30 IU per single human dose. When one-dilution tests are performed, the potency of the test vaccine is demonstrated to be significantly greater than 30 IU per human dose.

By IU measurements, compositions generally include at least 30 IU/dose. Compositions typically include between 20 and 80 Lf/ml of diphtheria toxoid, typically about 50 Lf/ml. Booster vaccines for adolescents and adults typically contain between 4 Lf/ml and 8 Lf/ml of diphtheria toxoid, e.g., 2.5 Lf, preferably 4 Lf, per 0.5 ml dose. Paediatric vaccines typically contain between 20 and 50 Lf/ml of diphtheria toxoid, e.g. 10 Lf or 25 Lf per 0.5 ml dose.

Purity of a protein preparation can be expressed by the ratio of specific protein to total protein. The purity of diphtheria toxoid in a composition is generally expressed in units of Lf diphtheria toxoid per unit mass of protein (nondialysable) nitrogen. For instance, a very pure toxin/toxoid might have a purity of more than 1700 Lf/mg N, indicating that most or all of the protein in the composition is diphtheria toxin/toxoid [19].

Tetanus

Clostridium tetani causes tetanus. Tetanus toxin can be treated to give a protective toxoid. The toxoids are used in tetanus vaccines and are well known in the art. Thus, a combination vaccine of the invention can include a tetanus toxoid. Preferred tetanus toxoids are those prepared by formaldehyde treatment. The tetanus toxoid can be obtained by growing C. tetani in growth medium (e.g. a Latham medium derived from bovine casein), followed by formaldehyde treatment, ultrafiltration and precipitation. The growth medium for growing C. tetani may be free from animal-derived components. The material may then be treated by a process comprising sterile filtration and/or dialysis.

The tetanus toxoid may be adsorbed onto an adjuvant, for example, an aluminium salt adjuvant.

Quantities of tetanus toxoid can be expressed in ‘Lf’ units (see below), defined as the amount of toxoid which, when mixed with one International Unit of antitoxin, produces an optimally flocculating mixture [14]. The NIBSC supplies ‘The 2nd International Reference Reagent for Tetanus Toxoid For Flocculation Test’ (NIBSC Code: 04/150) which contains 690 LF per ampoule, by which measurements can be calibrated.

Booster vaccines for adolescents and adults typically contain 5 Lf of tetanus toxoid per 0.5 ml dose. Paediatric vaccines typically contain between 5 and 10 Lf of tetanus toxoid per 0.5 ml dose.

The immunizing potency of tetanus toxoid is measured in international units (IU), assessed by comparing the protection afforded by a composition in laboratory animals (typically guinea pigs) with a reference vaccine e.g. using NIBSC's ‘Tetanus Toxoid Adsorbed Third International Standard 2000’ [20,21], which contains 469 IU per ampoule. The potency of tetanus toxoid in a composition of the invention should be at least 35 U per dose e.g. at least 70 IU/ml. More particularly, the potency of tetanus toxoid in a composition of the invention is at least 40 IU per dose. However, in booster vaccines for adults and adolescents, a reduced potency of 20 IU/dose may be acceptable because of the reduced antigen content in comparison to paediatric vaccine intended for primary immunization.

A multiple dilution assay can be used to determine the potency of the compositions of the invention. After immunization, guinea-pigs are bled or challenged either by the subcutaneous or by the intradermal route. As an alternative, mice may be used in place of guinea pigs. When guinea pigs or mice are bled, the antitoxin levels of the individual animals are titrated by means of toxin neutralization tests performed using in vivo or in vitro serological methods that have been validated on vaccines of the types being tested. The potency of the composition of the invention is calculated using appropriate statistical methods. Where multiple dilution assays are used, the lower and upper limits of 95% confidence interval should be within 50-200% of the estimated potency respectively. The lower 95% confidence limit of the estimated potency of a tetanus vaccine used for the primary immunization of children should not be less than 40 IU per single human dose.

The purity of tetanus toxoid in a composition is generally expressed in units of Lf tetanus toxoid per unit mass of protein (non-dialyzable) nitrogen. The tetanus toxoid should have a purity of at least 1000 Lf/mg N.

Hib

Haemophilus influenzae type b (‘Hib’) causes bacterial meningitis. Hib vaccines are typically based on the capsular saccharide antigen the preparation of which is well documented in the art. The H. influenzae bacteria can be cultured in the absence of animal-derived components. The Hib saccharide is conjugated to a carrier protein in order to enhance its immunogenicity, especially in children. Typical carrier proteins in these conjugates are tetanus toxoid, diphtheria toxoid, the CRM197 derivative of diphtheria toxin, or an outer membrane protein complex (OMPC) from serogroup B. meningococcus. Thus, a combination vaccine of the invention can include a Hib capsular saccharide conjugated to a carrier protein.

Any suitable activation chemistry and/or linker chemistry can be used in the conjugation of Hib saccharides. The saccharide will typically be activated or functionalised prior to conjugation. Activation may involve, for example, cyanylating reagents such as CDAP (e.g. 1-cyano-4-dimethylamino pyridinium tetrafluoroborate [30, 31]). Other suitable techniques use carbodiimides, hydrazides, active esters, norborane, p-nitrobenzoic acid, N-hydroxysuccinimide, S-NHS, EDC, TSTU; see also the introduction to reference 32).

Linkages via a linker group may be made using any known procedure, for example, the procedures described in references 33 and 34. One type of linkage involves reductive amination of the polysaccharide, coupling the resulting amino group with one end of an adipic acid linker group, and then coupling a protein to the other end of the adipic acid linker group [35, 36, 37]. Other linkers include B-propionamido [38], nitrophenyl-ethylamine [39], haloacyl halides [40], glycosidic linkages [41, 42, 43], 6-aminocaproic acid [44], ADH [45], C₄ to C₁₂ moieties [46] etc. As an alternative to using a linker, direct linkage can be used. Direct linkages to the protein may comprise oxidation of the polysaccharide followed by reductive amination with the protein, as described in, for example, references 46 and 47.

Tetanus toxoid may be used in the conjugate commonly referred to as ‘PRP-T’. PRP-T can be made by activating a Hib capsular polysaccharide using cyanogen bromide, coupling the activated saccharide to an adipic acid linker (such as (1-ethyl-3-(3-dimethylaminopropyl) carbodiimide), typically the hydrochloride salt), and then reacting the linker-saccharide entity with a tetanus toxoid carrier protein.

The CRM197 diphtheria toxoid is another preferred Hib carrier protein [47,48,49]. A preferred conjugate comprises the Hib saccharide covalently linked to CRM197 via adipic acid succinic diester [50,51].

The saccharide moiety of the conjugate may comprise full-length polyribosylribitol phosphate (PRP) as prepared from Hib bacteria, and/or fragments of full-length PRP. Conjugates with a saccharide:protein ratio (w/w) of between 1:5 (i.e. excess protein) and 5:1 (i.e. excess saccharide) may be used e.g. ratios between 1:2 and 5:1 and ratios between 1:1.25 and 1:2.5.

In preferred vaccines, however, the weight ratio of saccharide to carrier protein is between 1:2.5 and 1:3.5. In vaccines where tetanus toxoid is present both as an antigen and as a carrier protein then the weight ratio of saccharide to carrier protein in the conjugate may be between 1:0.3 and 1:2 [52].

Quantities of Hib antigens are typically expressed in μg of saccharide. The concentration of saccharide in a vaccine is typically between from 3 to 30 μg/ml e.g. 20 μg/ml. Administration of the Hib conjugate preferably results in an anti-PRP antibody concentration of ≥0.15 μg/ml, and more preferably ≥1 μg/ml, and these are the standard response thresholds. In some embodiments of the invention, the Hib polyribosylribitol phosphate is synthetic, for example, as described in [53 or 54].

Hepatitis B Virus

Hepatitis B virus (HBV) is a cause of viral hepatitis. The HBV virion consists of an inner core surrounded by an outer protein coat or capsid, and the core contains the viral DNA genome. The major component of the capsid is a protein known as HBV surface antigen or, more commonly, ‘HBsAg’, which is typically a 226-amino acid polypeptide with a molecular weight of ˜24 kDa. All existing hepatitis B vaccines contain HBsAg, and when this antigen is administered to a normal patient, it stimulates the production of anti-HBsAg antibodies which protect against HBV infection. Thus, immunogenic compositions of the invention can include HBsAg.

For vaccine manufacture, HBsAg can be made in a number of ways. For example, by expressing the protein by recombinant DNA methods. HBsAg for use with the method of the invention should be recombinantly expressed, e.g. in yeast cells. Suitable yeasts include Saccharomyces (such as S. cerevisiae), Hanensula (such as H. polymorpha) or Pichia hosts. The yeasts can be cultured in the absence of animal-derived components. Yeast-expressed HBsAg is generally non-glycosylated, and this is the most preferred form of HBsAg for use in immunogenic compositions of the invention. Yeast-expressed HBsAg is highly immunogenic and can be prepared without the risk of blood product contamination. Many methods for purifying HBsAg from recombinant yeast are known in the art.

The HBsAg will generally be in the form of substantially-spherical particles (average diameter of about 20 nm), including a lipid matrix comprising phospholipids. Yeast-expressed HBsAg particles may include phosphatidylinositol, which is not found in natural HBV virions. The particles may also include a non-toxic amount of LPS in order to stimulate the immune system [55]. The particles may retain non-ionic surfactant (e.g. polysorbate 20) if this was used during disruption of yeast [56].

The HBsAg is preferably from HBV subtype adw2.

A preferred method for HBsAg purification involves, after cell disruption: ultrafiltration; size exclusion chromatography; anion exchange chromatography; ultracentrifugation; desalting; and sterile filtration. Lysates may be precipitated after cell disruption (e.g. using a polyethylene glycol), leaving HBsAg in solution, ready for ultrafiltration.

After purification HBsAg may be subjected to dialysis (e.g. with cysteine), which can be used to remove any mercurial preservatives such as thimerosal that may have been used during HBsAg preparation [57].

Quantities of HBsAg are typically expressed in micrograms. Combination vaccines containing HBsAg usually include between 5 and 60 μg/ml. The concentration of HBsAg in a composition of the invention is preferably less than 60 μg/ml e.g. ≤55 μg/ml, ≤50 μg/ml, ≤45 μg/ml, ≤40 μg/ml, etc. A concentration of about 20 μg/ml is typical e.g. 10 μg per dose. In some embodiments of the invention, a composition includes a ‘low dose’ of HBsAg. This means that the concentration of HBsAg in the composition is ≤5 μg/ml e.g. <4, <3, <2.5, <2, <1, etc. In a typical 0.5 ml unit dose volume, therefore, the amount of HBsAg is less than 2.5 μg e.g. <2, <1.5, <1, <0.5, etc.

Poliovirus

Poliovirus causes poliomyelitis. Inactivated polio virus vaccine (IPV), as disclosed in more detail in chapter 24 of reference 1, has been known for many years. Thus, a combination vaccine of the invention can include an inactivated poliovirus antigen.

Polioviruses may be grown in cell culture, and a preferred culture uses a Vero cell line, derived from monkey kidney. Vero cells can conveniently be cultured microcarriers. After growth, virions may be purified using techniques such as ultrafiltration, diafiltration, and chromatography. Where animal (and particularly bovine) materials are used in the culture of cells, they should be obtained from sources that are free from transmissible spongiform encephalopathies (TSEs), and in particular free from bovine spongiform encephalopathy (BSE). Preferably, polioviruses are grown in cells cultured in medium free of animal-derived components.

Prior to administration to patients, polioviruses must be inactivated, and this can be achieved by treatment with formaldehyde (or, preferably, a non-aldehyde agent). Poliomyelitis can be caused by one of three types of poliovirus. The three types are similar and cause identical symptoms, but they are antigenically very different and infection by one type does not protect against infection by others. It is therefore preferred to use three poliovirus antigens with the invention: poliovirus Type 1 (e.g. Mahoney strain), poliovirus Type 2 (e.g. MEF-1 strain), and poliovirus Type 3 (e.g. Saukett strain). Other strains of poliovirus Type 1, Type 2 and Type 3 are known in the art and may also be used. The viruses are preferably grown, purified and inactivated individually, and are then combined to give a bulk trivalent mixture for use with the invention.

Quantities of IPV are typically expressed in the ‘DU’ unit (the “D-antigen unit” [58]). Combination vaccine usually comprise between 1-100 DU per polioviral type per dose e.g., about 40 DU of type 1 poliovirus, about 8 DU of type 2 poliovirus, and about 32 DU of type 3 poliovirus, but it is possible to use lower doses than these [59,60] e.g. 10-20 DU for type 1, 2-4 DU for type 2, and 8-20 DU for type 3. A combination vaccine of the invention can include a ‘low dose’ of a poliovirus. For a Type 1 poliovirus this means that the concentration of the virus in the composition is ≤20 DU/ml e.g. <18, <16, <14, <12, <10, etc. For a Type 2 poliovirus this means that the concentration of the virus in the composition is ≤4 DU/ml e.g. <3, <2, <1, <0.5, etc. For a Type 3 poliovirus this means that the concentration of the virus in the composition is ≤16 DU/ml e.g. <14, <12, <10, <8, <6, etc. Where all three of Types 1, 2 and 3 polio virus are present the three antigens can be present at a DU ratio of 5:1:4 respectively, or at any other suitable ratio e.g. a ratio of 15:32:45 when using Sabin strains [61]. A low dose of antigen from Sabin strains is particularly useful, with ≤10 DU type 1, ≤20 DU type 2, and ≤30 DU type 3 (per unit dose, typically 0.5 ml).

Where an IPV component is used, and the polioviruses were grown on Vero cells, a vaccine composition preferably contains less than 10 ng/ml, preferably ≤1 ng/ml e.g. ≤500 μg/ml or ≤50 μg/ml of Vero cell DNA e.g. less than 10 ng/ml of Vero cell DNA that is ≥50 base pairs long.

Preparing a Combination Vaccine

Antigenic components from these pathogens for use in vaccines are commonly referred to by abbreviated names: ‘D’ for diphtheria toxoid; ‘T’ for tetanus toxoid; ‘P’ for pertussis antigens, with ‘aP’ being acellular (e.g. including at least OMVs of the invention, PT and FHA and optionally pertactin and/or FIM2/FIM3); HBsAg for hepatitis B surface antigen; ‘Hib’ for conjugated H. influenzae b capsular saccharide; and ‘IPV’ for 3-valent inactivated poliovirus.

Embodiments of the invention include, but are not limited to combination vaccines comprising the following components:
D, T, aP
D, T, aP, IPV
D, T, aP, HBsAg
D, T, aP, Hib
D, T, aP, Hib, IPV
D, T, aP, HBsAg, Hib
D, T, aP, HBsAg, IPV
D, T, aP, HBsAg, IPV, Hib

These combination vaccines may consist essentially of only the antigens listed, or may further include antigens from additional pathogens. Particularly, these combination vaccines contain only the antigens listed as active ingredients but may further comprise excipients such as adjuvants, buffers and the like. In some embodiments the aP component consists of OMVs of the invention, PT (preferably genetically detoxified PT) and FHA. In some embodiments the aP component consists of OMVs of the invention, PT (preferably genetically detoxified PT), FHA and PRN. In some embodiments the aP component consists of OMVs of the invention, PT (preferably genetically detoxified PT), FHA and FIM2/FIM3. In some embodiments the aP component consists of OMVs of the invention, PT (preferably genetically detoxified PT), FHA, PRN and FIM2/FIM3.

For paediatric combination vaccines, the ratio of D:T is typically greater than 1 (i.e. paediatric vaccines usually have excess D in Lf units) and generally between 2:1 and 3:1 (measured in Lf units), e.g. 2.5:1. In contrast, for booster vaccine that are administered to adolescents or adults (who usually have received at least one paediatric combination vaccine comprising D and T), the ratio of T:D is typically greater than 1 (i.e. booster vaccines usually have excess T in Lf units) and generally between 1.5:1 and 2.5:1, e.g. 2:1.

One useful vaccine includes OMVs of the invention and, per unit dose, 2Lf D, 5Lf T, 4 μg PT-9K/129G, 4 μg FHA and 8 μg pertactin. Another useful vaccine includes OMVs of the invention and, per unit dose, 25Lf D, 10Lf T, 25 μg PT-9K/129G, 25 μg FHA and 8 μg pertactin.

When combining antigenic components to prepare a multivalent composition, the antigens can be added individually, or they can be pre-mixed. Where a combination vaccine comprises D and T antigens and additional antigens, a pre-mixed D-T component can be used in the preparation of the combination vaccine. This bivalent component can be combined with further antigens. Where a D-T mixture is used for preparing a combination vaccine, the ratio of diphtheria toxoid to tetanus toxoid in the mixture can be between 2:1 and 3:1 (measured in Lf units), preferably between 2.4:1 and 2.6:1, e.g. preferably 2.5:1.

Methods of Treatment

In a fourth aspect of the invention, the OMVs or immunogenic composition may be used to induce an immune response in a mammal, particularly a suitable mammal. The suitable mammal is preferably a human; the immune response is preferably protective and preferably involves antibodies.

Immunogenic and pharmaceutical compositions of the invention are for in vivo use for eliciting an immune response against an immunogen of interest. As disclosed herein, methods for raising an immune response in a mammal comprising the step of administering an effective amount of an immunogenic composition or pharmaceutical composition of the invention are provided. The immune response is preferably protective and preferably involves antibodies and/or cell mediated immunity. The method may raise a booster response.

The invention also provides an immunogenic composition or pharmaceutical composition of the invention for use in a method for raising an immune response in a mammal.

The invention also provides the use of an OMV or population of OMVs of the invention in the manufacture of a medicament for raising an immune response in a mammal.

By raising an immune response in the mammal by these uses and methods, the mammal can be protected against various diseases and/or infections e.g. against bacterial and/or viral diseases as discussed above. The OMVs and compositions are immunogenic, and are more preferably vaccine compositions. Vaccines according to the invention may either be prophylactic (i.e. to prevent infection) or therapeutic (i.e. to treat infection), but will typically be prophylactic.

The mammal, particularly a suitable mammal may be a human or a large veterinary mammal (e.g. horses, cattle, deer, goats, and pigs). Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc.

Vaccines prepared according to the invention may be used to treat both children and adults. Thus, a human patient may be less than 1 year old, less than 5 years old, 1-5 years old, 5-15 years old, 15-55 years old, or at least 55 years old. In some embodiments, the child is an individual under one year of age, for example, less than one day old, about 1 week old, about 2 weeks old, about 3 weeks old, about 4 weeks old, about 2 months old, about 3 months, about 4 months, about 5 months, about 6 months, about 7 months, about 8 months old, about 9 months old, about 10 months old, about 11 months old, less than about 12 months old).

Particular patient groups for receiving the vaccines are the elderly (e.g. 20 ˜50 years old, ˜60 years old, and preferably ˜65 years), the young (e.g. ˜5 years old or infants less than about 12 months old), hospitalised patients, healthcare workers, armed service and military personnel, pregnant women, the chronically ill, or immunodeficient patients. The vaccines are not suitable solely for these groups, however, and may be used more generally in a population.

Compositions of the invention will generally be administered directly to a patient. Direct delivery may be accomplished by parenteral injection (e.g. subcutaneously, intraperitoneally, intravenously, intramuscularly, intradermally, or to the interstitial space of a tissue. Alternative delivery routes include rectal, oral (e.g. tablet, spray), buccal, sublingual, vaginal, topical, transdermal or transcutaneous, intranasal, ocular, aural, pulmonary or other mucosal administration. Intradermal and intramuscular administration are two preferred routes. Injection may be via a needle (e.g. a hypodermic needle), but needle-free injection may alternatively be used. A typical intramuscular dose is 0.5 mL.

The invention may be used to elicit systemic and/or mucosal immunity, preferably to elicit an enhanced systemic and/or mucosal immunity.

These uses and methods are preferably for the prevention and/or treatment of a disease caused by Bordetella pertussis and optionally one or more of Corynebacterium diphtheriae, Clostridium tetani, Haemophilus influenzae serotype b, polio virus and hepatitis B virus. In one preferred embodiment, these uses and methods are for the prevention of a disease caused by Bordetella pertussis.

The subject in which disease is prevented may not be the same as the subject that receives the immunogenic composition of the invention. For instance, an immunogenic composition may be administered to a female (before or during pregnancy) in order to protect offspring (so-called ‘maternal immunization’). The immunization of the pregnant female provides antibody-mediated immunity to the infant through passive immunity resulting from transferred maternal antibody (‘passive maternal immunity’). The passive immunity occurs naturally when maternal antibodies are transferred to the foetus through the placenta. Passive immunity is especially important to infants because they are born without any actively acquired immunity. Administration of compositions of the invention to a pregnant female enhances immunity in the female, and antibodies are passed to the new-born through the placenta, conferring passive maternal immunity on the infant. However, passive immunity in infants is only temporary and starts to decrease after the first few weeks, or months of life. As passive immunity is only temporary, it may be important for the infant to receive administration of a composition of the invention, to induce active immunity in the infant, before the passive immunity diminishes. Administration of a second immunogenic composition to the infant after birth induces active immunity in the infant, and extends the immunity passed on from the mother during pregnancy.

Dosage can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.). In one embodiment, multiple doses may be administered approximately 6 weeks, 10 weeks and 14 weeks after birth, e.g. at an age of 6 weeks, 10 weeks and 14 weeks, as often used in the World Health Organisation's Expanded Program on Immunisation (“EPI”). In an alternative embodiment, two primary doses are administered about two months apart, e.g. about 7, 8 or 9 weeks apart, followed by one or more booster doses about 6 months to 1 year after the second primary dose, e.g. about 6, 8, 10 or 12 months after the second primary dose. In a further embodiment, three primary doses are administered about two months apart, e.g. about 7, 8 or 9 weeks apart, followed by one or more booster doses about 6 months to 1 year after the third primary dose, e.g. about 6, 8, 10, or 12 months after the third primary dose.

Methods to Modulate Reactogenicity of Lipid A

In a fifth aspect, the invention relates to a method of modulating the reactogenicity of the Lipid A of a Bordetella pertussis bacterium, comprising stably integrating into the genome of the bacterium at least one gene selected from the group consisting of LpxA and LpxD, wherein:

(i) the LpxA gene encodes an LpxA protein having at least 80% sequence identity with SEQ ID NO: 1 or 2 and wherein the protein comprises Serine at position 170 (S170) and/or Alanine at position 229 (A229) when numbered in accordance with SEQ ID NO: 1 or 2; and/or

(ii) the LpxD gene encodes an LpxD protein having at least 90% amino acid sequence identity with SEQ ID NO:3 or SEQ ID NO: 4. As described above, the reactogenicity of lipid A of a gram-negative bacterium, particularly a B. pertussis bacterium, can be modulated by expression of exogenous enzymes from the lipid A biosynthetic pathway, for example acetyltransferases LpxA and LpxD.

Genomic integration of the LpxA and LpxD genes allows for the stable expression of the exogenous DNA without the need for antibiotic selection. In addition, surprisingly the Inventors discovered that such strains do not suffer growth defects seen in the art.

### Definitions

As used in the present disclosure and claims, the singular forms “a,” “an,” and “the” include plural forms unless the context clearly dictates otherwise; i.e., “a” means “one or more” unless indicated otherwise.

The term “and/or” as used in a phrase such as “A and/or B” is intended to include “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “comprising” encompasses “including” e.g. a composition “comprising” X may include something additional e.g. X+Y. The word “substantially” does not exclude “completely” e.g. a composition which is “substantially free” from Y may be completely free from Y. In some implementations, the term “comprising” refers to the inclusion of the indicated active agent, such as recited polypeptides, as well as inclusion of other active agents, and pharmaceutically acceptable carriers, excipients, emollients, stabilizers, etc., as are known in the pharmaceutical industry. In some implementations, the term “consisting essentially of” refers to a composition, whose only active ingredient is the indicated active ingredient(s), for example antigens, however, other compounds may be included which are for stabilizing, preserving, etc. the formulation, but are not involved directly in the therapeutic effect of the indicated active ingredient. Use of the transitional phrase “consisting essentially” means that the scope of a claim is to be interpreted to encompass the specified materials or steps recited in the claim, and those that do not materially affect the basic and novel characteristic(s) of the claimed invention. See, In re Herz, 537 F.2d 549, 551-52, 190 USPQ 461, 463 (CCPA 1976) (emphasis in the original); see also MPEP § 2111.03. Thus, the term “consisting essentially of” when used in a claim of this invention is not intended to be interpreted to be equivalent to “comprising”.

The term “consisting of” and variations thereof means “limited to” unless expressly specified otherwise. In certain territories, the term “comprising an active ingredient consisting of” may be used in place of “consisting essentially”.

The term “about” in relation to a numerical value x means within an acceptable contextual error range for the particular value as determined by one of ordinary skill in the art, which will depend in part on how the value is measured or determined, i.e. the limitations of the measurement system or the degree of precision required for a particular purpose, for example, x±10%, x±5%, x±4%, x±3%, x±2%, x±1%.

The word “substantially” does not exclude “completely” e.g. a composition which is “substantially free” from Y may be completely free from Y but may encompass conditions that function in all important aspects as free conditions but where the numerical values indicate the presence of some impurities or substances. Where necessary, the word “substantially” may be omitted from the definition of the invention. Where particular values are used in the specification and in the claims, unless otherwise stated, the term “substantially” means with an acceptable error range for the particular value.

Where methods refer to steps of administration, for example as (a), (b), (c), etc., these are intended to be sequential, i.e., step (c) follows step (b) which is preceded by step (a).

Antibodies will generally be specific for their target, i.e., they will have a higher affinity for the target than for an irrelevant control protein, such as bovine serum albumin.

All references, patents or patent applications cited within this patent specification are incorporated by reference herein.

### SPECIFIC EMBODIMENTS OF THE INVENTION

Embodiment 1. A recombinant Bordetella pertussis bacterium which comprises:

(i) at least one genomic LpxA gene encoding an LpxA protein, wherein the LpxA protein comprises a mutation at position 170 and/or a mutation at position 229 relative to SEQ ID NO: 1; and/or

(ii) at least one genomic insertion of a heterologous LpxD gene.

Embodiment 2. The recombinant Bordetella pertussis bacterium of Embodiment 1 which comprises at least one genomic LpxA gene encoding an LpxA protein, wherein the LpxA protein comprises a substitution at position 170 and/or a substitution at position 229 relative to SEQ ID NO: 1.

Embodiment 3. The recombinant Bordetella pertussis bacterium of Embodiment 1 or 2 wherein the LpxA protein comprises a Serine residue at position 170 and/or an Alanine residue at position 229 relative to SEQ ID NO: 1.

Embodiment 4. The recombinant Bordetella pertussis bacterium of Embodiment 3, wherein the LpxA protein comprises (i) a Serine residue at position 170 and (ii) an Alanine residue at position 229 when numbered in accordance with SEQ ID NO:1.

Embodiment 5. The recombinant Bordetella pertussis bacterium of Embodiment 4 which comprises an LpxA gene that encodes an LpxA protein having at least 80% amino acid sequence identity to SEQ ID NO: 1 or 2.

Embodiment 6. The recombinant Bordetella pertussis bacterium of any preceding Embodiment which comprises an LpxA gene encoding an LpxA protein, wherein the LpxA gene is derived from Bordetella parapertussis (LpxA_{Bpa}).

Embodiment 7. The recombinant Bordetella pertussis bacterium of any preceding Embodiment which comprises an LpxA gene encoding an LpxA protein, wherein the LpxA protein has SEQ ID NO: 2.

Embodiment 8. The recombinant Bordetella pertussis bacterium of any preceding Embodiment, wherein the heterologous LpxD gene encodes an LpxD protein having at least 90% amino acid sequence identity with SEQ ID NO:3 or SEQ ID NO: 4.

Embodiment 9. The recombinant Bordetella pertussis bacterium of Embodiment 8, wherein the heterologous LpxD gene encodes an LpxD protein having at least 95% amino acid sequence identity with SEQ ID NO:3 or SEQ ID NO: 4.

Embodiment 10. The recombinant Bordetella pertussis bacterium of Embodiment 9, wherein the heterologous LpxD gene encodes an LpxD protein having an amino acid sequence selected from the group consisting of SEQ ID NO:3 or SEQ ID NO: 4.

Embodiment 11. The recombinant Bordetella pertussis bacterium of any preceding Embodiment, wherein the endogenous LpxA gene is inactivated and/or the endogenous LpxD gene is inactivated.

Embodiment 12. The recombinant Bordetella pertussis bacterium of any one of the preceding Embodiments which produces lipid A wherein (i) at least 30% of the C3′ acyl chains are from about C10 to about C12 in length; and/or (ii) at least 30% of the C2′ acyl chains are from about C10 to about C12 in length; and/or (iii) at least 30% of the C2 acyl chains are from about C10 to about C12 in length.

Embodiment 13. The recombinant Bordetella pertussis bacterium of any one of the preceding Embodiments which produces lipid A wherein (i) at least 30% of the C3′ acyl chains are from C10 to about C12 in length.

Embodiment 14. The recombinant Bordetella pertussis bacterium of any one of the preceding Embodiments which produces lipid A wherein (i) at least 30% of the C3′ acyl chains are C10 in length.

Embodiment 15. The recombinant Bordetella pertussis bacterium of any one of the preceding Embodiments which produces lipid A wherein substantially all of C3′ acyl chains of the Lipid A have a length of C10.

Embodiment 16. The recombinant Bordetella pertussis bacterium of any one of the preceding Embodiments that produces Lipid A with reduced endotoxic activity compared to that of the Lipid A produced by the parental strain, particularly when measured using TLR4 stimulation assays, particularly human TLR4 stimulation assays.

Embodiment 17. The recombinant Bordetella pertussis bacterium of any one of the preceding Embodiments, wherein the growth curve of a bacterial population of said bacteria is comparable to the growth curve of a population of the parental strain.

Embodiment 18. The recombinant Bordetella pertussis bacterium of any one of the preceding Embodiments wherein the bacterium is derived from the Tohama I strain and expresses the genetically detoxified pertussis toxoid PT-9K/129G.

Embodiment 19. An isolated outer membrane vesicle (OMV) derived from the recombinant Bordetella pertussis bacterium of any one of Embodiments 1 to 18 which comprises modified Lipid A incorporated into the membrane wherein substantially all of C3′ acyl chains of the Lipid A have a length of C10 and/or wherein substantially all of C2 and C2′ acyl chains of the Lipid A have a length of C12.

Embodiment 20. The isolated outer membrane vesicle according to Embodiment 19 which comprises modified Lipid A incorporated into the membrane wherein substantially all of C3′ acyl chains of the Lipid A have a length of C10.

Embodiment 21. The isolated outer membrane vesicle according to Embodiment 19 or 20 which is substantially free of dermonecrotic toxin and/or which contains endogenous genetically detoxified pertussis toxoid.

Embodiment 22. An immunogenic composition comprising at least one isolated OMV according to Embodiment 19 to 21 and a pharmaceutically acceptable excipient.

Embodiment 23. The immunogenic composition of Embodiment 22, further comprising at least one additional antigen selected from the group consisting of (1) pertussis toxoid (PT), (2) FHA, (3) pertactin (PRN), (4) FIM2/FIM3, (5) adenylate cyclase, (6) diphtheria toxoid (DT), (7) tetanus toxoid (TT), (8) inactivated polio virus (IPV), (9) hepatitis B surface antigen and (10) Hib PRP.

Embodiment 24. The OMV of Embodiment 19 to 21 or the immunogenic composition of Embodiment 22 or 23 for use in inducing an immune response in a suitable mammal, for example a human.

Embodiment 25. The OMV of Embodiment 19 to 21 or the immunogenic composition of Embodiment 22 or 23 for use in prophylaxis or as a vaccine, for example, for use in prophylaxis of disease caused by Bordetella pertussis

Embodiment 26. The OMV of Embodiment 19 to 21 or the immunogenic composition of Embodiment 22 or 23 for use in prophylaxis of disease caused by Bordetella pertussis.

Embodiment 27. A method of modulating the reactogenicity of the Lipid A of a Bordetella pertussis bacterium, comprising stably integrating into the genome of the bacterium at least one gene selected from the group consisting of LpxA and LpxD, wherein:

(i) the LpxA gene encodes an LpxA protein having at least 80% sequence identity with SEQ ID NO: 1 or 2 and wherein the protein comprises Serine at position 170 (S170) and/or Alanine at position 229 (A229) when numbered in accordance with SEQ ID NO: 1 or 2; and/or

(ii) the LpxD gene encodes an LpxD protein having at least 90% amino acid sequence identity with SEQ ID NO:3 or SEQ ID NO: 4.

Embodiment 28. A recombinant Bordetella pertussis bacterium which comprises at least one genomic LpxA gene encoding an LpxA protein, wherein the LpxA protein comprises a Serine residue at position 170 and an Alanine residue at position 229 (relative to SEQ ID NO: 1) and wherein the LpxA gene encodes an amino acid sequence having at least 80% identity to SEQ ID NO: 1 or 2.

Embodiment 29. A recombinant Bordetella pertussis bacterium which comprises at least one episomal LpxA gene encoding an LpxA protein, wherein the LpxA protein comprises a Serine residue at position 170 and an Alanine residue at position 229 (relative to SEQ ID NO: 1) and wherein the LpxA gene encodes an amino acid sequence having at least 80% identity to SEQ ID NO: 1 or 2.

Embodiment 30. A recombinant Bordetella pertussis bacterium which comprises (i) at least one genomic LpxA gene encoding an LpxA protein, wherein the LpxA protein comprises a Serine residue at position 170 and an Alanine residue at position 229 (relative to SEQ ID NO: 1) and wherein the LpxA gene encodes an amino acid sequence having at least 80% identity to SEQ ID NO: 1 or 2 and (ii) at least one genomic insertion of a heterologous LpxD gene wherein the heterologous LpxD gene encodes an amino acid sequence having at least 95% sequence identity with SEQ ID NO:3 or SEQ ID NO: 4.

Embodiment 31. A recombinant Bordetella pertussis bacterium which comprises at least one episomal LpxA gene encoding an LpxA protein, wherein the LpxA protein comprises a Serine residue at position 170 and an Alanine residue at position 229 (relative to SEQ ID NO: 1) and wherein the LpxA gene encodes an amino acid sequence having at least 80% identity to SEQ ID NO: 1 or 2 and (ii) at least one genomic insertion of a heterologous LpxD gene wherein the heterologous LpxD gene encodes an amino acid sequence having at least 95% sequence identity with SEQ ID NO:3 or SEQ ID NO: 4.

Embodiment 32. A recombinant Bordetella pertussis bacterium which comprises at least one genomic insertion of a heterologous LpxD gene wherein the heterologous LpxD gene encodes an amino acid sequence having at least 95% sequence identity with SEQ ID NO:3 or SEQ ID NO: 4.

Embodiment 33. A recombinant Bordetella pertussis bacterium which comprises at least one genomic LpxA gene encoding an LpxA protein, wherein the LpxA protein comprises a Serine residue at position 170 and an Alanine residue at position 229 (relative to SEQ ID NO: 1) and wherein the LpxA gene encodes an amino acid sequence having at least 80% identity to SEQ ID NO: 1 or 2 and wherein the bacterium is derived from the Tohama strain and expresses the genetically detoxified pertussis toxoid PT-9K/129G.

Embodiment 34. A recombinant Bordetella pertussis bacterium which comprises at least one episomal LpxA gene encoding an LpxA protein, wherein the LpxA protein comprises a Serine residue at position 170 and an Alanine residue at position 229 (relative to SEQ ID NO: 1) and wherein the LpxA gene encodes an amino acid sequence having at least 80% identity to SEQ ID NO: 1 or 2 and wherein the bacterium is derived from the Tohama I strain and expresses the genetically detoxified pertussis toxoid PT-9K/129G.

Embodiment 35. A recombinant Bordetella pertussis bacterium which comprises (i) at least one genomic LpxA gene encoding an LpxA protein, wherein the LpxA protein comprises a Serine residue at position 170 and an Alanine residue at position 229 (relative to SEQ ID NO: 1) and wherein the LpxA gene encodes an amino acid sequence having at least 80% identity to SEQ ID NO: 1 or 2 and (ii) at least one genomic insertion of a heterologous LpxD gene wherein the heterologous LpxD gene encodes an amino acid sequence having at least 95% sequence identity with SEQ ID NO:3 or SEQ ID NO: 4 and wherein the bacterium is derived from the Tohama I strain and expresses the genetically detoxified pertussis toxoid PT-9K/129G.

Embodiment 36. A recombinant Bordetella pertussis bacterium which comprises at least one episomal LpxA gene encoding an LpxA protein, wherein the LpxA protein comprises a Serine residue at position 170 and an Alanine residue at position 229 (relative to SEQ ID NO: 1) and wherein the LpxA gene encodes an amino acid sequence having at least 80% identity to SEQ ID NO: 1 or 2 and (ii) at least one genomic insertion of a heterologous LpxD gene wherein the heterologous LpxD gene encodes an amino acid sequence having at least 95% sequence identity with SEQ ID NO:3 or SEQ ID NO: 4 and wherein the bacterium is derived from the Tohama I strain and expresses the genetically detoxified pertussis toxoid PT-9K/129G.

Embodiment 37. A recombinant Bordetella pertussis bacterium which comprises at least one genomic insertion of a heterologous LpxD gene wherein the heterologous LpxD gene encodes an amino acid sequence having at least 95% sequence identity with SEQ ID NO:3 or SEQ ID NO: 4 and wherein the bacterium is derived from the Tohama I strain and expresses the genetically detoxified pertussis toxoid PT-9K/129G.

Embodiment 38. A recombinant Bordetella pertussis bacterium which comprises at least one genomic LpxA gene encoding an LpxA protein, wherein the LpxA protein comprises a Serine residue at position 170 and an Alanine residue at position 229 (relative to SEQ ID NO: 1) and wherein the LpxA gene encodes an amino acid sequence having at least 80% identity to SEQ ID NO: 1 or 2 and wherein the bacterium is derived from the Tohama I strain, expresses the genetically detoxified pertussis toxoid PT-9K/129G and does not express the dermonectrotic toxin (DNT) gene (SEQ ID NO: 19).

Embodiment 39. A recombinant Bordetella pertussis bacterium which comprises at least one episomal LpxA gene encoding an LpxA protein, wherein the LpxA protein comprises a Serine residue at position 170 and an Alanine residue at position 229 (relative to SEQ ID NO: 1) and wherein the LpxA gene encodes an amino acid sequence having at least 80% identity to SEQ ID NO: 1 or 2 and wherein the bacterium is derived from the Tohama I strain, expresses the genetically detoxified pertussis toxoid PT-9K/129G and does not express the dermonectrotic toxin (DNT) gene (SEQ ID NO: 19).

Embodiment 40. A recombinant Bordetella pertussis bacterium which comprises (i) at least one genomic LpxA gene encoding an LpxA protein, wherein the LpxA protein comprises a Serine residue at position 170 and an Alanine residue at position 229 (relative to SEQ ID NO: 1) and wherein the LpxA gene encodes an amino acid sequence having at least 80% identity to SEQ ID NO: 1 or 2 and (ii) at least one genomic insertion of a heterologous LpxD gene wherein the heterologous LpxD gene encodes an amino acid sequence having at least 95% sequence identity with SEQ ID NO:3 or SEQ ID NO: 4 and wherein the bacterium is derived from the Tohama I strain, expresses the genetically detoxified pertussis toxoid PT-9K/129G and does not express the dermonectrotic toxin (DNT) gene (SEQ ID NO: 19).

Embodiment 41. A recombinant Bordetella pertussis bacterium which comprises at least one episomal LpxA gene encoding an LpxA protein, wherein the LpxA protein comprises a Serine residue at position 170 and an Alanine residue at position 229 (relative to SEQ ID NO: 1) and wherein the LpxA gene encodes an amino acid sequence having at least 80% identity to SEQ ID NO: 1 or 2 and (ii) at least one genomic insertion of a heterologous LpxD gene wherein the heterologous LpxD gene encodes an amino acid sequence having at least 95% sequence identity with SEQ ID NO:3 or SEQ ID NO: 4 and wherein the bacterium is derived from the Tohama I strain, expresses the genetically detoxified pertussis toxoid PT-9K/129G and does not express the dermonectrotic toxin (DNT) gene (SEQ ID NO: 19).

Embodiment 42. A recombinant Bordetella pertussis bacterium which comprises at least one genomic insertion of a heterologous LpxD gene wherein the heterologous LpxD gene encodes an amino acid sequence having at least 95% sequence identity with SEQ ID NO:3 or SEQ ID NO: 4 and wherein the bacterium is derived from the Tohama I strain, expresses the genetically detoxified pertussis toxoid PT-9K/129G and does not express the dermonectrotic toxin (DNT) gene (SEQ ID NO: 19).

Embodiment 43. An isolated outer membrane vesicle (OMV) derived from the recombinant Bordetella pertussis bacterium of any one of Embodiments 28 to 42 which comprises modified Lipid A incorporated into the membrane wherein substantially all of C3′ acyl chains of the Lipid A have a length of C10 and/or wherein substantially all of C2 and C2′ acyl chains of the Lipid A have a length of C12.

Embodiment 44. An isolated outer membrane vesicle (OMV) derived from the recombinant Bordetella pertussis bacterium of any one of Embodiments 28 to 42 which comprises modified Lipid A incorporated into the membrane wherein substantially all of C3′ acyl chains of the Lipid A have a length of C10.

Embodiment 45. An immunogenic composition, particularly a pharmaceutical composition such as a vaccine, comprising an immunologically effective amount of isolated OMVs according to Embodiment 43 or 44 and a pharmaceutically acceptable carrier.

Embodiment 46. The recombinant Bordetella pertussis bacterium of any one of Embodiments 1 to 18 wherein the bacterium comprises genomic insertion of a heterologous LpxE gene.

Embodiment 47. The recombinant Bordetella pertussis bacterium of Embodiment 46 wherein the heterologous LpxE gene is from Rhizobium leguminosarum (LpxE_{R}i).

Embodiment 48. The recombinant Bordetella pertussis bacterium of Embodiment 47 wherein the amino acid sequence encoded by the heterologous LpxE gene is SEQ ID NO:6.

Embodiment 49. The recombinant Bordetella pertussis bacterium of Embodiment 46 wherein the heterologous LpxE gene is from Francisella novicida (LpxE_{F}n).

Embodiment 50. The recombinant Bordetella pertussis bacterium of Embodiment 49 wherein the amino acid sequence encoded by the heterologous LpxE gene is SEQ ID NO:7.

Embodiment 51. The recombinant Bordetella pertussis bacterium of any one of Embodiments 46 to 50 wherin the heterologous LpxE gene is under the control of the BP0840 promoter.

Embodiment 52. The recombinant Bordetella pertussis bacterium of any one of Embodiments 46 to 51 wherein the heterologous LpxE gene is inserted into the bacterial genome.

Embodiment 53. The recombinant Bordetella pertussis bacterium of Embodiment 52 wherein the heterologous LpxE gene replaces the locus of the ArnT gene (ΔArnT).

Embodiment 54. The recombinant Bordetella pertussis bacterium of any one of Embodiments 1 to 18 wherein the bacterium comprises genomic insertion of a heterologous LpxF gene.

Embodiment 55. The recombinant Bordetella pertussis bacterium of Embodiment 54 wherein the heterologous LpxF gene is from Francisella novicida (LpxF_{Fn}).

Embodiment 56. The recombinant Bordetella pertussis bacterium of Embodiment 55 wherein the amino acid sequence encoded by the heterologous LpxF gene is SEQ ID NO:8.

Embodiment 57. The recombinant Bordetella pertussis bacterium of any one of Embodiments 54 to 56 where the heterologous LpxF gene is under the control of the BP0840 promoter.

Embodiment 58. The recombinant Bordetella pertussis bacterium of any one of Embodiments 54 to 57 wherein the heterologous LpxF gene is inserted into the bacterial genome.

Embodiment 59. The recombinant Bordetella pertussis bacterium of Embodiment 58 wherein the heterologous LpxF gene replaces the locus of the ArnT gene (ΔArnT).

### MODES FOR CARRYING OUT THE INVENTION

Bacterial Strains and Gene Sequences

Tables 1 to 3 list the key bacterial strains, DNA and amino acid sequences used:

TABLE 1
Bacterial strains and description

| Strain | Description |
|---|---|
| Tohama I PTg | Wild type B. pertussis Tomaha I which expresses |
| | the genetically detoxified pertussis toxoid PT- |
| | 9K/129G (Tohama I PTg) |
| ΔLpxA_{Bpe}/LpxA_{Bpa} | Tohama I with genomic integration of LpxA_{Bpa} and |
| | knock-out of endogenous LpxA_{Bpe} genes |
| ΔArnT/ΔLpxA_{Bpe}/ | Tohama I with genomic integration of LpxA_{Bpa} and |
| LpxA_{Bpa} | knock-out of endogenous LpxA_{Bpe} and ArnT |
| ΔLpxA_{Bpe}/LpxA_{Bpa}/ | Tohama I with genomic integration of LpxA_{Bpa} and |
| ΔDNT/LpxD_{Pa} | LpxD_{Pa} and knock-out endogenous LpxA_{Bpe} and |
| | DNT genes |
| LpxA_{Bpa/}ΔLpxA_{Bpe}/ | Tohama I with genomic integration of LpxA_{Bpa} and |
| ΔDNT/LpxD_{Pa}/ | LpxD_{Pa} and knock-out LpxA_{Bpe}, LpxD_{Bpe} and DNT |
| ΔLpxD_{Bpe} | genes |
| ΔArnT/PagL_{Bbr} | Tohama I with genomic integration of PagL and |
| | knock-out of endogenous ArnT gene |
| LpxD_{Pa} | Tohama I with episomal expression of LpxD from |
| | P. aeruginosa |
| LpxA_{Bpa} | Tohama I with episomal expression of LpxA from |
| | B. parapertussis |
| LpxE_{Fn} | Tohama I with episomal expression of LpxE from |
| | Francisella novicida |
| LpxE_{Rl} | Tohama I with episomal expression of LpxE from |
| | Rhizobium leguminosarum |
| LpxE_{Fn}/ΔArnT | Tohama I with genomic insertion and expression of |
| | LpxE from Francisella novicida at the locus of the |
| | endogenous ArnT gene. |
| LpxE_{Fn}/ΔArnT/ | Tohama I with genomic integration of LpxA_{Bpa}, |
| ΔLpxA_{Bpe}/LpxA_{Bpa} | genomic integration of LpxE_{Fn} and knock-out of |
| | endogenous LpxA_{Bpe} and ArnT |
| Tohama I | Wild type B. pertussis Tomaha I (ATCC BAA-589/ |
| | NCTC 13251) |

TABLE 2
Key amino acid sequences

| Sequence | | |
|---|---|---|
| ID | Amino acid sequence | Ref.: ID |
| 1 | LpxA Bordetella pertussis | UniProt: Q7VYB8 |
| 2 | LpxA Bordetella parapertussis | GenBank: BBH97344 |
| 3 | LpxD Comamonas testosteroni | GenBank: WP 012839123 |
| 4 | LpxD Pseudomonas aeruginosa | GenBank: WP 003098585 |
| 5 | PagL Bordetella bronchiseptica | GenBank: CAE35745 |
| 6 | LpxE Francisella novicida | UniProt: Q66RM6 |
| 7 | LpxE Rhizobium leguminosarum | UniProt: A0A2K9Z9T7 |
| 8 | LpxF Francisella novicida | UniProt: A0Q4N6 |

TABLE 3
Key DNA sequences

| Sequence | | |
|---|---|---|
| ID | Nucleic acid sequence | GenBank ID |
| 9 | LpxA Bordetella parapertussis | NC_018828.1: |
| | | 2648346-2649140 |
| 10 | LpxD Comamonas testosteroni | BBQP01000023.1: |
| | | 16517588 |
| 11 | LpxD Pseudomonas aeruginosa | LR590473.1: |
| | | 19088931909954 |
| 12 | LpxD Comamonas testosteroni (Codon | N/A |
| | optimised for expression in B. pertussis) | |
| 13 | LpxD Pseudomonas aeruginosa (Codon | N/A |
| | optimised for expression in B. pertussis) | |
| 14 | N-term insert for genomic integration | N/A |
| | into B. pertussis | |
| 15 | C-term insert for genomic integration | N/A |
| | into B. pertussis | |
| 16 | PagL Bordetella bronchiseptica | BX640448: |
| | | 164131-163595 |
| 17 | Porin promoter (BP0840 promoter) | BX640413.1: |
| | | 174494-174789 |
| 18 | ArnT Bordetella pertussis | |
| 19 | DNT Bordetella pertussis | |
| 20 | Construct for ArnT KO | N/A |
| 21 | Construct for replacing DNT with LpxD | N/A |
| | P. aeruginosa | |
| 22 | LpxE Francisella novicida (Codon | N/A |
| | optimised for expression in B. pertussis) | |
| 23 | LpxE Rhizobium leguminosarum | N/A |
| | (Codon optimised for expression in | |
| | B. pertussis) | |
| 24 | LpxF Francisella novicida (Codon | N/A |
| | optimised for expression in B. pertussis) | |

Production and Culture of Recombinant Bacterial Strains

The heterologous Lpx genes were expressed in Bordetella pertussis either by genomic integration or by episomal (non-genomic) expression.

Genomic Integration

Synthetic sequences containing the LpxA_{Bpa} sequence of interest (SEQ ID NO: 9) and upstream and downstream homology arms to allow for homologous recombination (SEQ ID NO: 14 and 15) were cloned into vector pSORTP1 [62] using flanking EcoRI and HindIII restriction sites. Vector pSORTP1 contains an origin of conjugative transfer, gentamycin and ampicillin selection markers and a copy of the rpsL gene that confers sensitivity to streptomycin.

The pSORTP1 vectors were transformed into E. coli SM10 Apir and recombinant clones were co-cultivated with a nalidixic acid/streptomycin-resistant B. pertussis strain Tohama I to allow conjugative transfer of the vector. The streptomycin resistance of this strain is conferred by a mutation in the rpsL gene. The plasmid cannot replicate in B. pertussis and needs to be integrated into the bacterial genome by homologous recombination in order to confer gentamycin resistance. Recombinant B. pertussis were obtained by concurrent selection on gentamycin (to remove wild-type B. pertussis) and nalidixic acid (to remove E. coli SM10 donor strain). Genomic introduction of the pSORTP1 vector containing a functional copy of the rpsL gene sensitises B. pertussis to streptomycin. Selection on streptomycin forces removal of the vector backbone by a second homologous recombination event on the other side of the transgene to achieve a markerless mutation. Using this approach, we obtained a recombinant B. pertussis Tohama I strain expressing the LpxA sequence from B. parapertussis. A number of additional strains were constructed as follows:

In strain ΔArnT/PagL_{Bbr} the ArnT locus in the B. pertussis genome was replaced with a copy of the B. bronchiseptica PagL gene under the control of the promoter region of B. pertussis BP0840 (outer membrane porin). A recombinant sequence was constructed with ArnT upstream and downstream regions containing the B. bronchiseptica PagL sequence. The recombinant sequence was cloned into vector pSORTP1 using flanking EcoRI and HindIII restriction sites. Following transformation, as described above, the resulting strain combines the knock-out of the ArnT gene and the expression of the PagL gene.

In strain ΔArnT/ΔLpxA_{Bpe}/LpxA_{Bpa} the ArnT locus (SEQ ID NO: 20) in the B. pertussis genome is knocked-out.

In strain ΔLpxA_{Bpe}/LpxA_{Bpa}/ADNT/LpxD_{Pa}/ΔLpxD_{Bpe}, a copy of LpxD_{Pa} was integrated into the genome (SEQ ID NO: 21), replacing the dermonecrotic toxin (DNT) gene (SEQ ID NO: 19). The wild-type LpxD_{Bp} was subsequently knocked out.

In strain LpxE_{Fn}/ΔArnT/ΔLpxA_{Bpe}/LpxA_{Bpa}, the ArnT locus (SEQ ID NO: 20) in the B. pertussis genome was replaced with a copy of the Francisella novicida LpxE gene (SEQ ID NO: 22) under the control of the promoter region of B. pertussis BP0840 (outer membrane porin SEQ ID NO: 17). A recombinant sequence was constructed with ArnT upstream and downstream regions containing the Francisella novicida LpxE sequence. The recombinant sequence was cloned into vector pSORTP1 using flanking EcoRI and HindIII restriction sites. Following transformation, as described above, the resulting strain combines the knock-out of the ArnT gene and the expression of the LpxE gene.

Episomal expression Synthetic sequences were cloned into vector pMMB67EH (ATCC Ref.: 37622) using flanking Acc65I and HindIII sites. Sequences encoding LpxD_{Ct}, LpxD_{Pa}, LpxE_{Fn}, LpxE_{Rl} and LpxF_{Fn} were codon optimized for expression in B. pertussis (PriorityGENE service, GENEWIZ).

The pMMB67EH vectors containing the heterologous LpxD, LpxE or LpxF genes were transformed into E. coli SM10 Apir and recombinant clones were co-cultivated with a nalidixic acid/streptomycin-resistant B. pertussis strain Tohama I to allow conjugative transfer of the vector. Recombinant B. pertussis containing the pMMB67EH plasmid was then selected on ampicillin and nalidixic acid. Stable transformation of B. pertussis Tohama I with the respective pMMB67EH constructs was confirmed by PCR detection of the heterologous Lpx sequence.

The B. pertussis recombinant strains were grown in commercially available media (see 63) in standard conditions; the medium was supplemented with 1 mM IPTG for episomal expression.

Growth Evaluation of Recombinant B. pertussis ΔLpxA_{Bpe}/LPxA_{Bpa}

In order to assess any potential impact on bacterial growth, recombinant strain ΔLpxA_{Bpe}/LpxA_{Bpa} was tested in a 10 L fermentation.

Briefly, two full Bordet-Gengou agar plates were used to inoculate two 30 mL, 24h pre-cultures. The 30 mL pre-cultures were then inoculated into two additional 24h pre-cultures of 1 L; these two second pre-cultures (approx. 1.5 L) were then used to inoculate the 10 L fermenter at OD_{650 nm} of 1.

A drop in the concentration of dissolved oxygen (DO) was observed at approximately 9 hours; this drop is associated with an increase in the oxygen consumption due to the exponential growth rate of the culture. The DO was then controlled by agitation speed.

The end of the fermentation process after 37.5 hours was characterized by a decrease of the stirring speed caused by a diminution of oxygen consumption, evidence of the depletion of the main carbon source (Na-L-glutamate). The optical density (OD_{650 nm}) at the time of harvest was 10.1, with a cell viability of 6.3 10¹⁰ (CFU/ml).

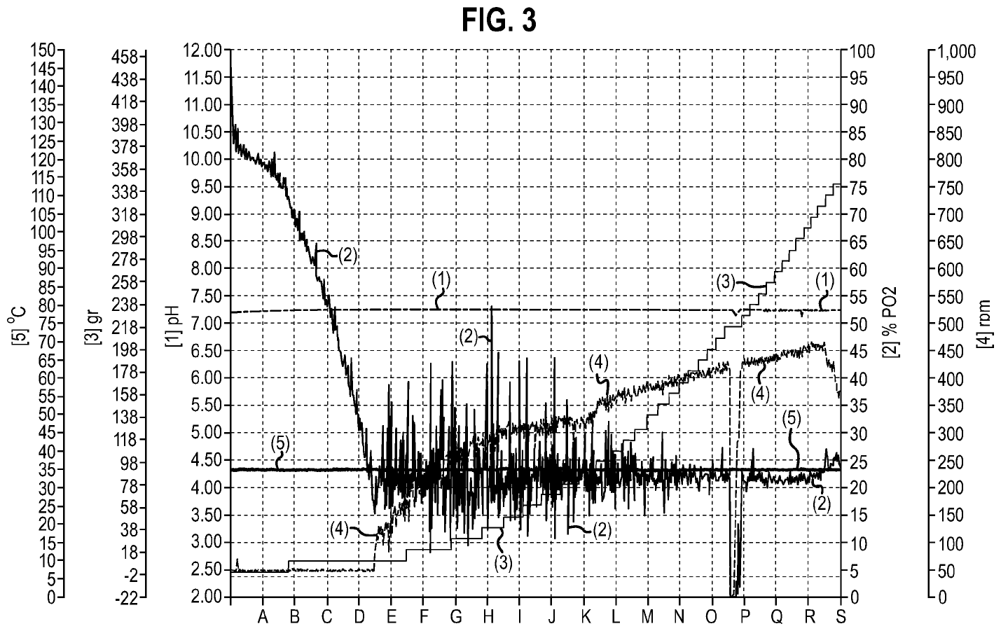

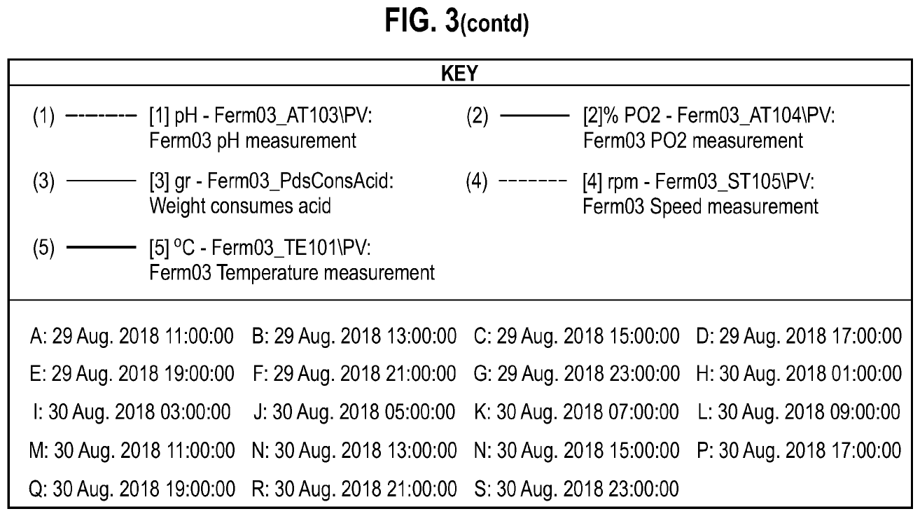

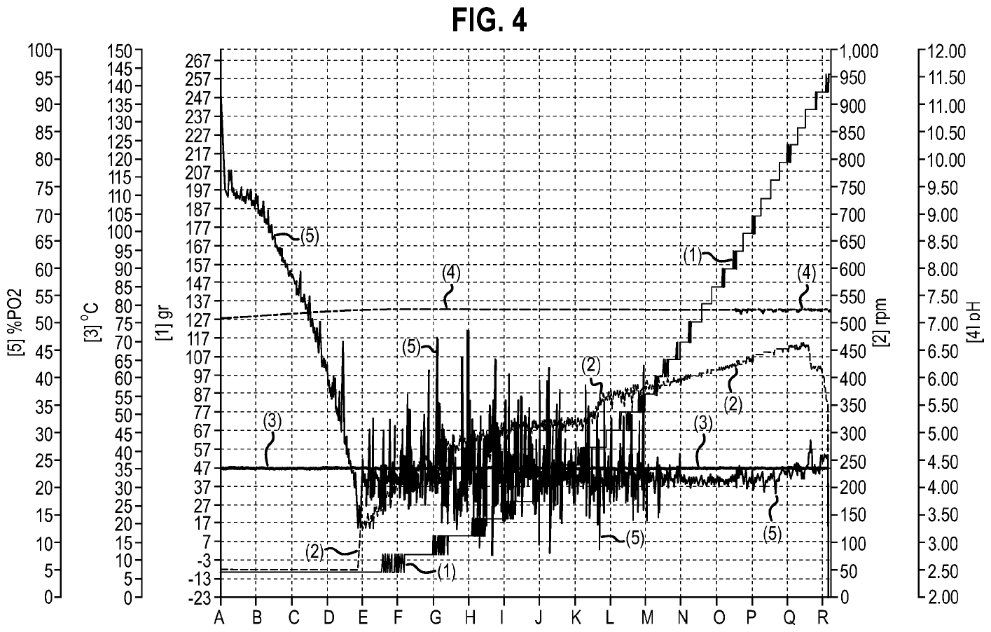

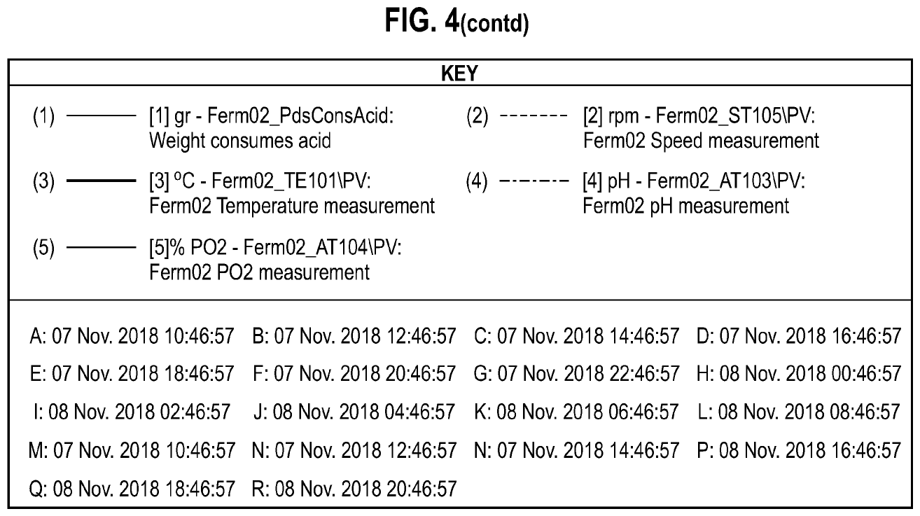

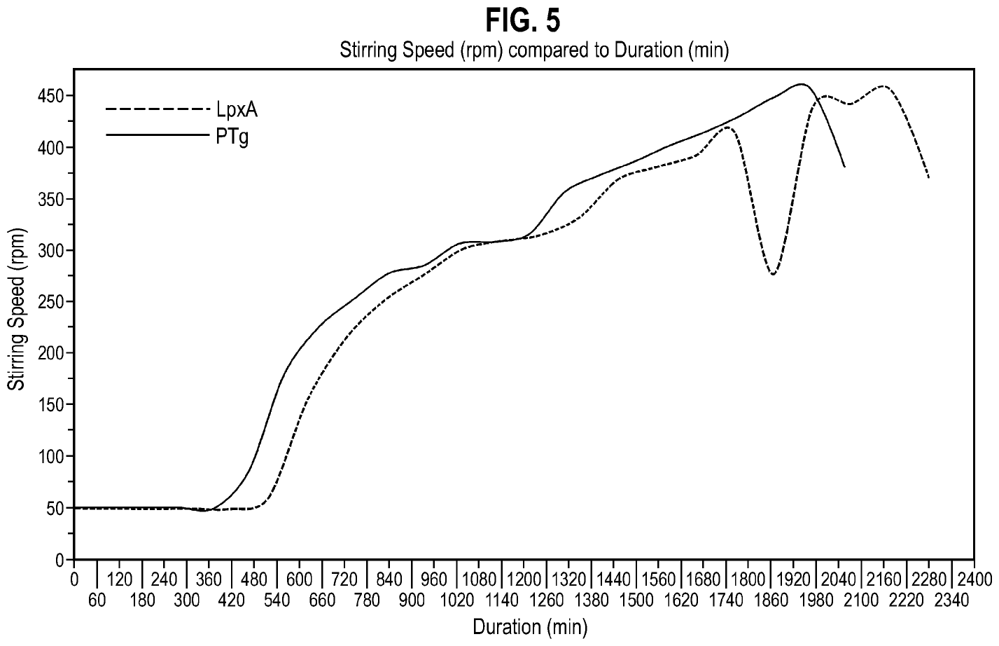

Overall, the fermentation profile of ΔLpxA_{Bpe}/LpxA_{Bpa} was comparable to that of the Tohama I PTg strain, with similar fermentation times and elapsed times to DO (Table 5, FIGS. 3 to 5).

TABLE 5
Fermentation parameters for B. pertussis
ΔLpxA_{Bpe}/LpxA_{Bpa} and Tohama I PTg.

| | | Mean values for |
|---|---|---|
| | ΔLpxA_{Bpe}/ | Tohama I PTg |
| | LpxA_{Bpa} | (n = 3) |
| Time elapsed to DO | 9 | 9.1 +/− 2.1 |
| regulation (h) | | |
| Fermentation duration (h) | 37.5 | 36.7 +/− 3.7 |
| Optical density (OD_{650 nm}) | 10.1 | 9.2 +/− 0.3 |
| Viability (CFU/ml) | 6.3 10⁺¹⁰ | 3.6 10⁺¹⁰ +/− 1.1 10⁺¹⁰ |

OMV Production

Outer membrane vesicles (OMV) were produced from bacterial cultures for mass spectrometric analysis of LPS structure as well as in vitro stimulation of the human TLR4 receptor. OMV were produced by detergent extraction from bacterial pellets after 24h culture.

Briefly, bacterial pellets were resuspended in 20 mM Tris-HCl, 2 mM EDTA and 50 U/mL benzonase, pH 8.6 and incubated for 30 min at room temperature. For detergent extraction of OMV, 0.1% DOC was added and the suspension was incubated for 30 min at 40° C. Cellular debris was removed by centrifugation at 20,000 g for 30 min (4° C.) and supernatant was sterile filtered (0.22 μm). Sterile supernatant was then ultracentrifuged (145,000 g, 2h, 4° C.), resuspended in 1×PBS (with 5 mM EDTA), ultracentrifuged a second time (same conditions), resuspended in 1×PBS (without EDTA) and sterile filtered (0.22 μm).

Characterization of LPS Structure by Mass Spectrometry

The OMV preparations were analysed by Liquid Chromatography Mass Spectrometry (LC-MS) to determine the impact of the genomic mutation of LpxA and/or LpxD (or LpxE) and the episomal expression of heterologous LpxA and/or LpxD, LpxE or LpxF genes on the structure of the LPS. OMV preparations from the wild-type strain Tohama I PTg were used as a control.

Briefly, OMV preparations were precipitated with 95% ethanol, pellets were dried and solubilized in 50% methanol and sonicated. Sonicated material was centrifuged and 5 μL of supernatant was injected on the HPLC column for MS analysis of LOS structure.

LpxA Recombinant Strains

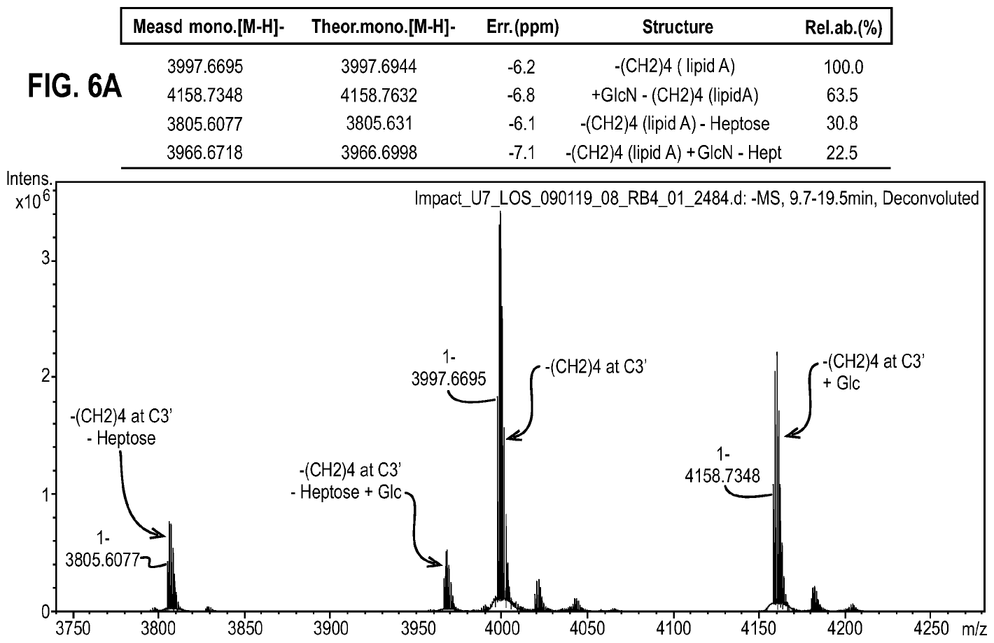

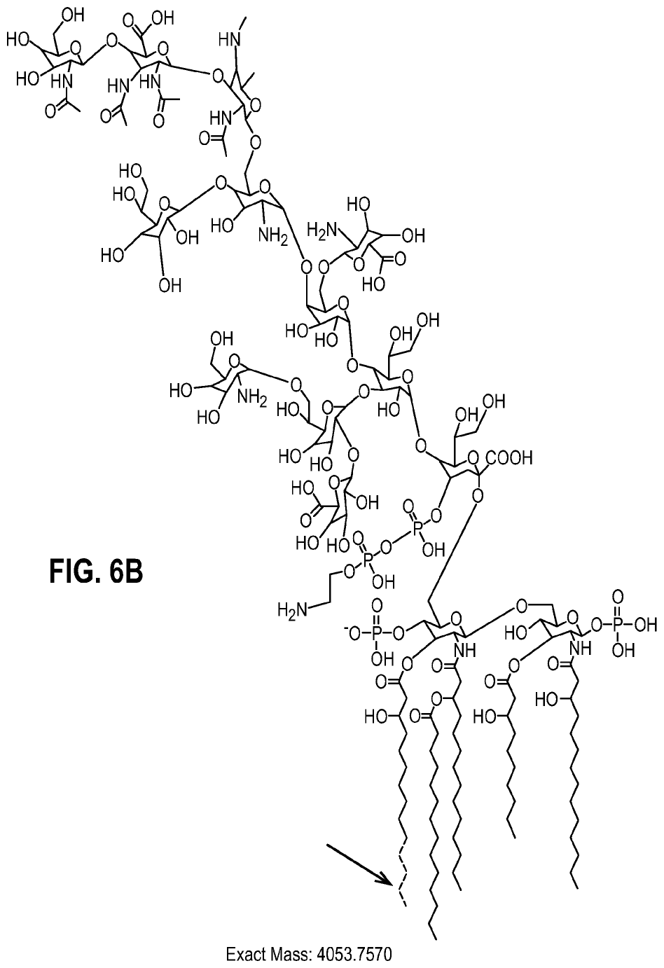

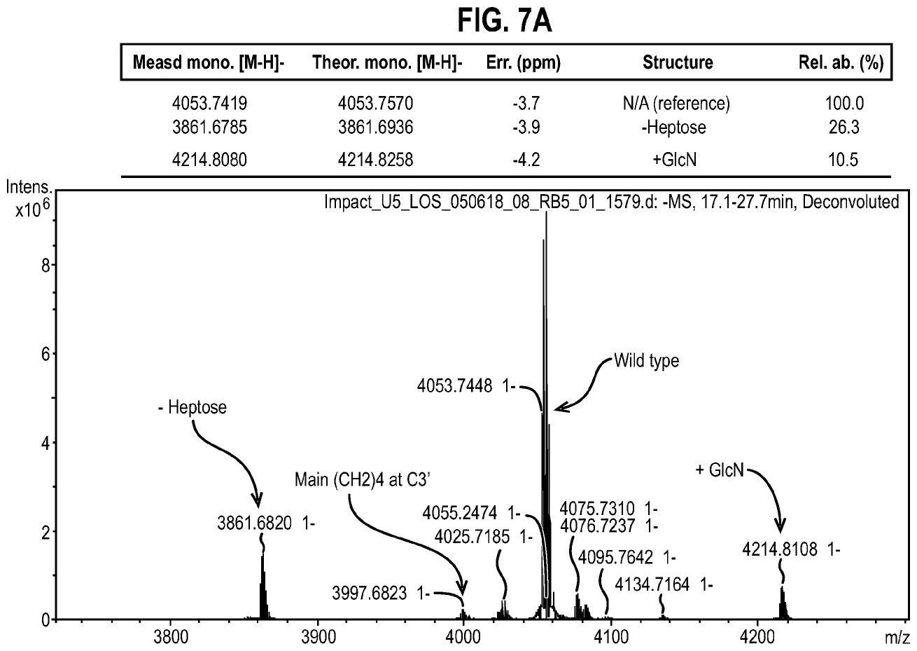

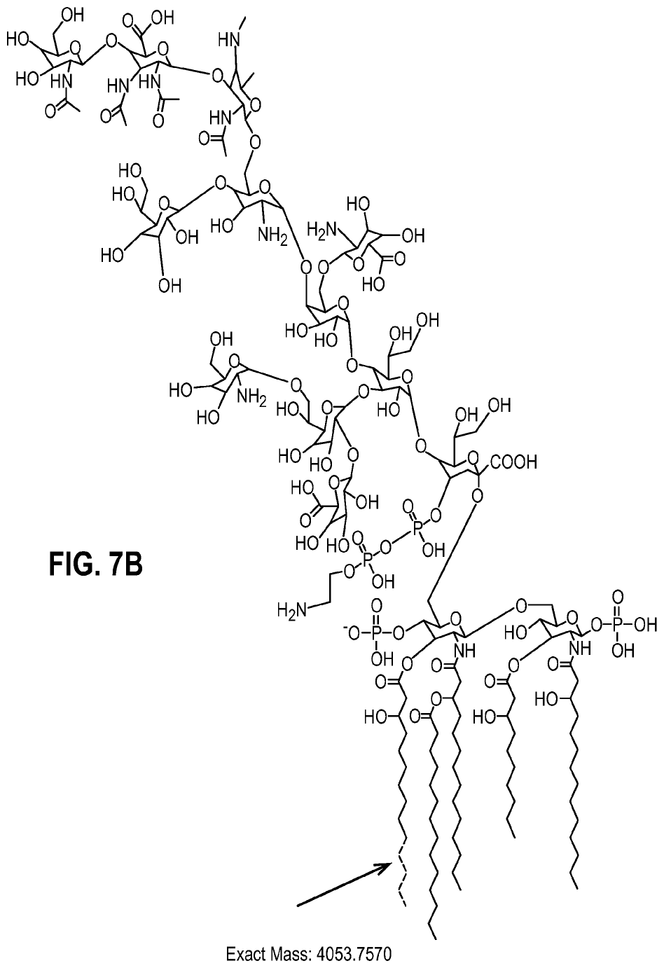

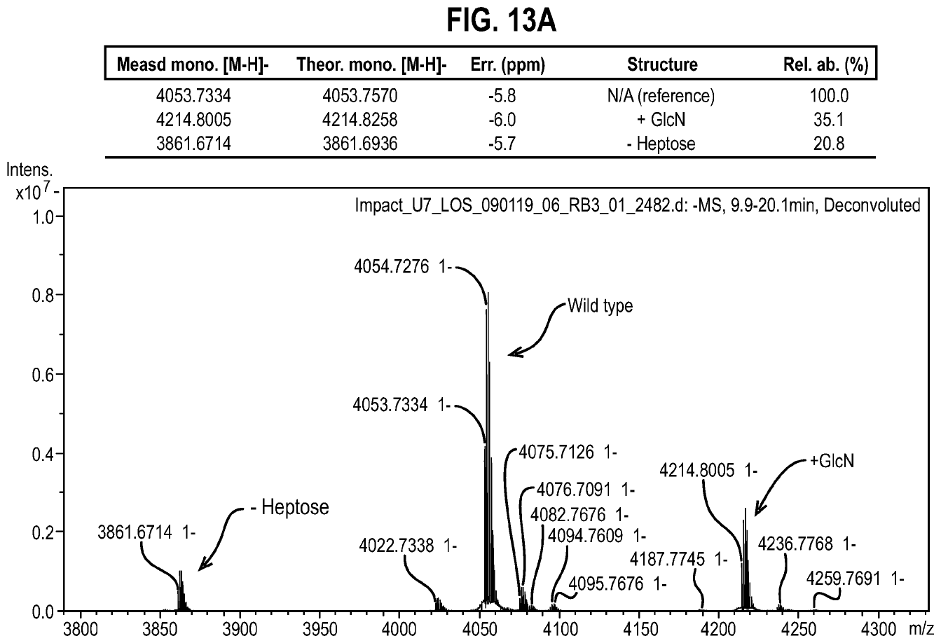

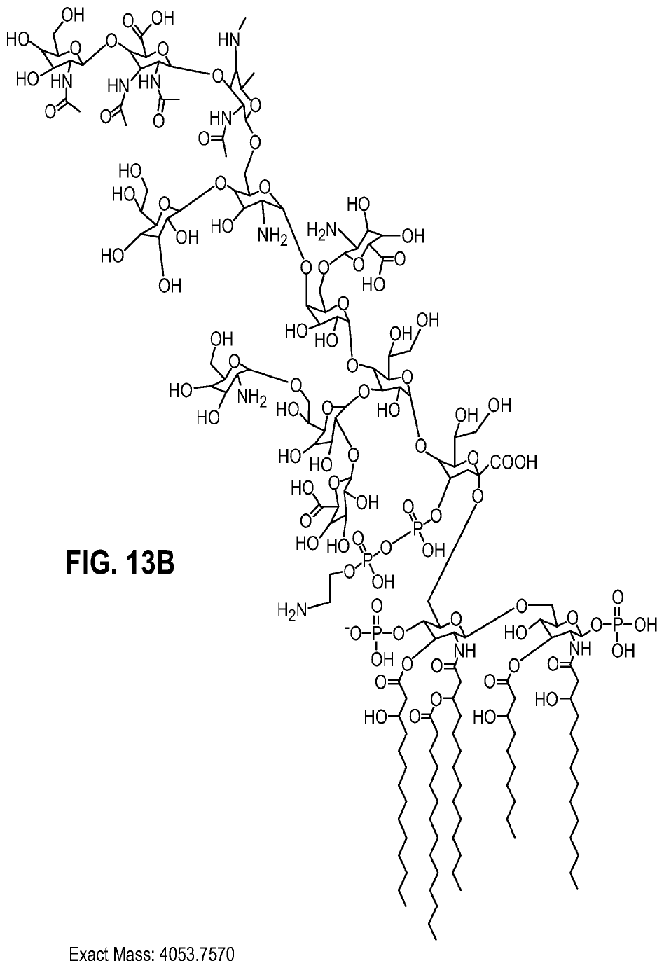

Genomic integration and expression of LpxA_{Bpa} had a significant impact on the LPS structure of the OMV from recombinant B. pertussis ΔLpxA_{Bpe}/LpxA_{Bpa}, with 100% of acyl chains at position C3′ displaying a length reduction from C₁₄ to C₁₀ (FIG. 6(B)—the dotted lines show the C₁₄ to C₁₀ reduction). In comparison, expression of episomal LpxA_{Bpa} in B. pertussis Tomaha I had a very low, unquantifiable impact (strain LpxA_{Bpa}); although the C₁₄ to C₁₀ variant is only present in a minority as for expression of episomal LpxA_{Bpa}, FIG. 7(B) still indicates where it can be different from wild-type (length reduction from C₁₄ to Cao). For reference, the structural analysis of LOS from wild type B. pertussis Tomaha I Ptg is shown in FIG. 13.

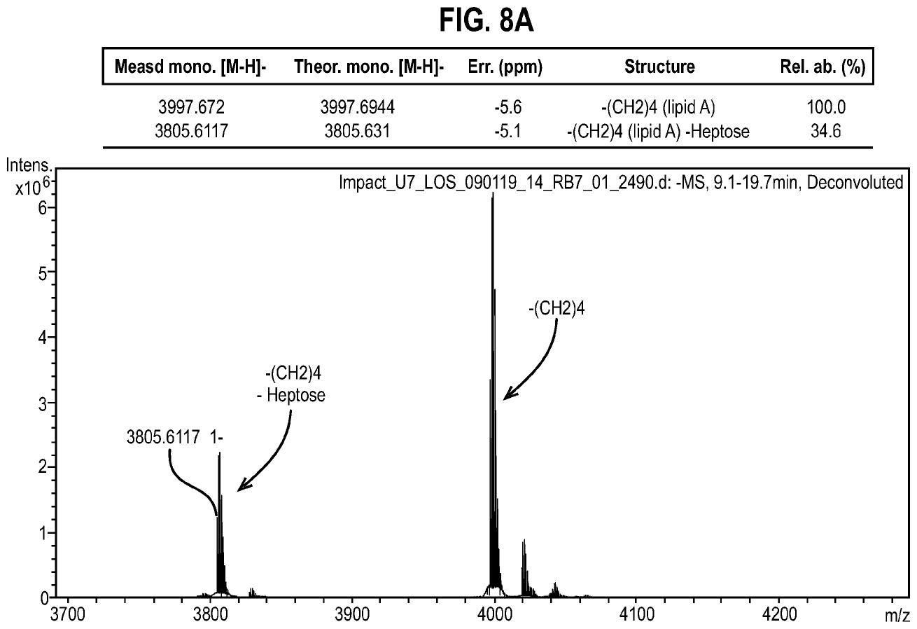

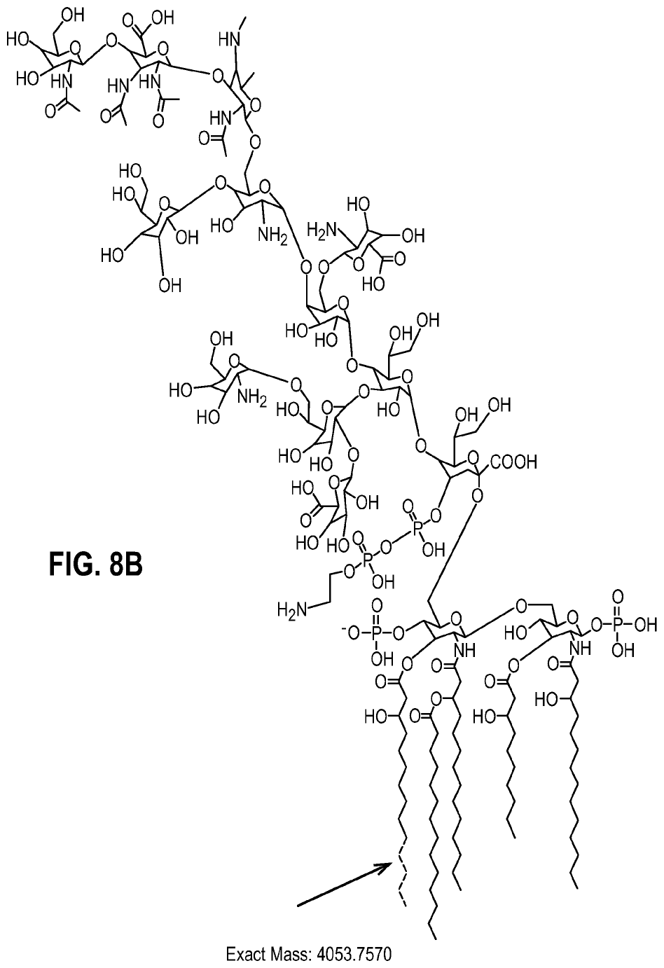

Genomic integration and expression of LpxA_{Bpa} had a similar effect on the LPS structure when combined with ΔArnT, with 100% of acyl chains at position C3′ displaying a length reduction from C₁₄ to C₁₀ (FIG. 8).

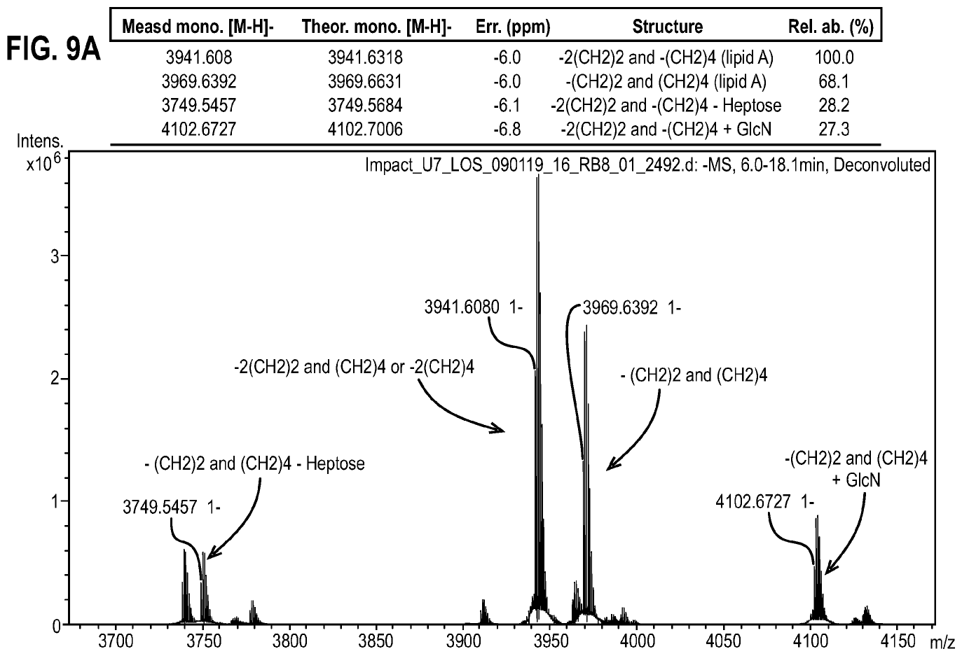

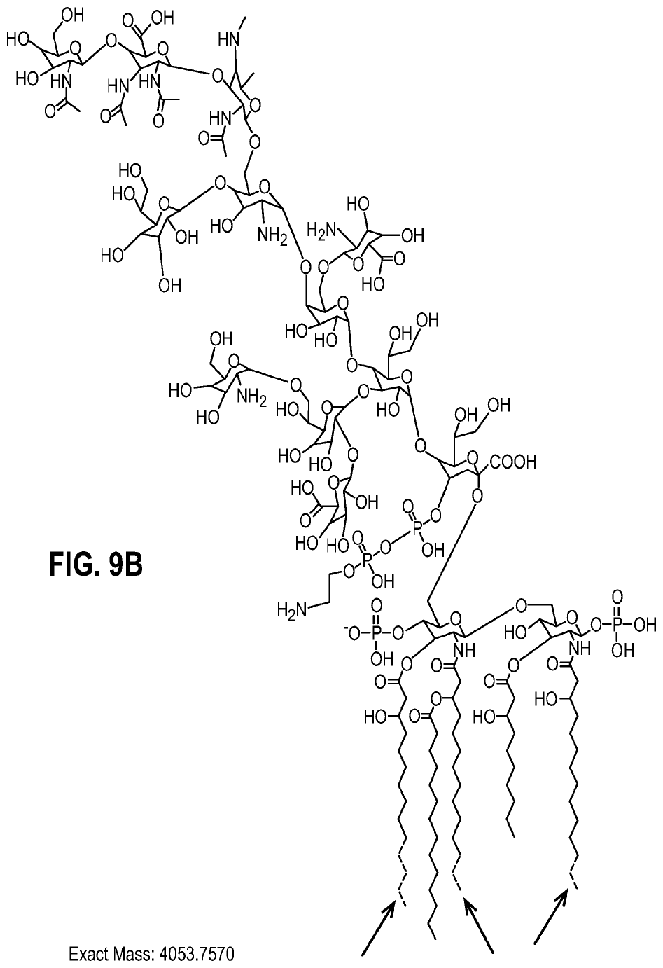

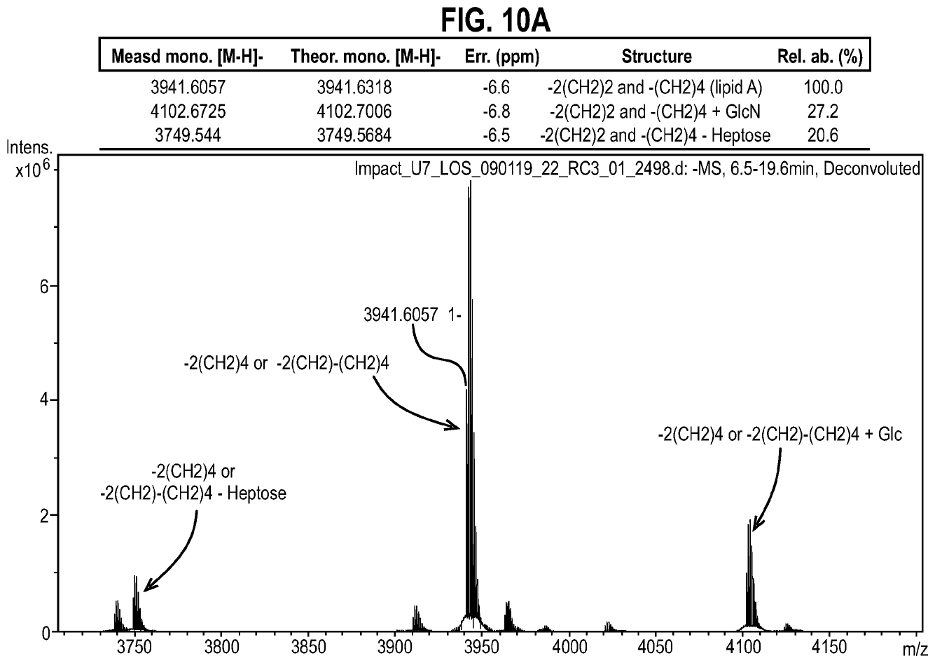

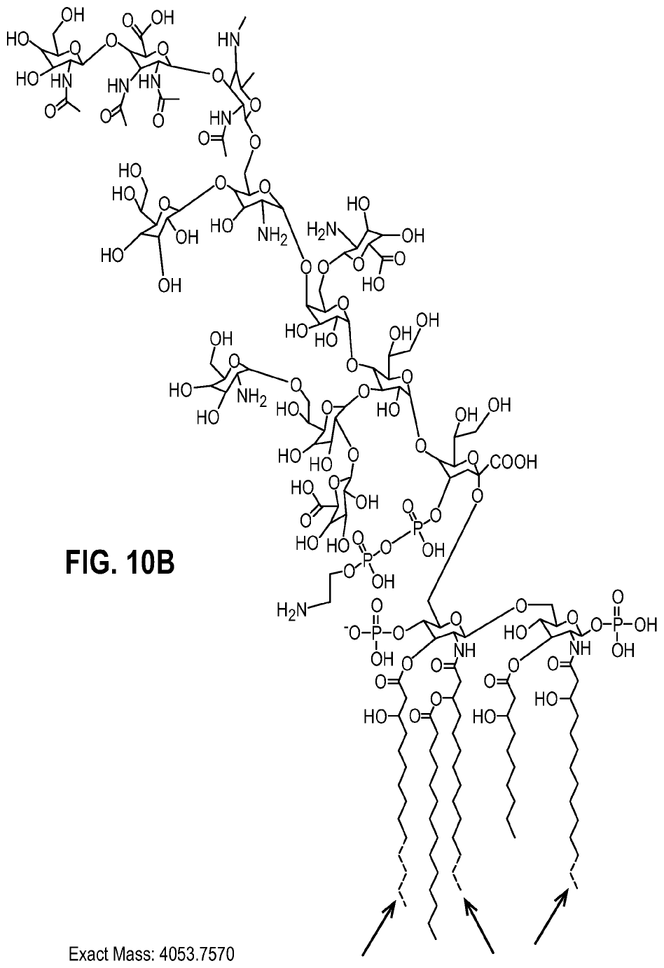

Importantly, genomic integration and expression of LpxA_{Bpa} also had the same effect on the length of the C3′ acyl chain in strains ΔLpxA_{Bpe}/LpxA_{Bpa}/ADNT/LpxD_{Pa} and ΔLpxA_{Bp}e/LpxA_{Bp}a/ADNT/LpxD_{P}a/ΔLpxD_{Bp}e, in which LpxD from Pseudomonas was also expressed (FIGS. 9 and 10). Expression of the exogenous LpxD gene, in combination with the inactivation of the respective endogenous LpxD had no detrimental effect on the activity of the exogenous LpxA gene. Additionally, no significant growth differences were observed.

Similarly, in strains in which LpxEFn was also expressed, the effect of genomic integration and expression of LpxABpa on the length of the C3′ acyl chain was not affected with 100% exhibiting a length of Cao.

LpxD Recombinant Strains

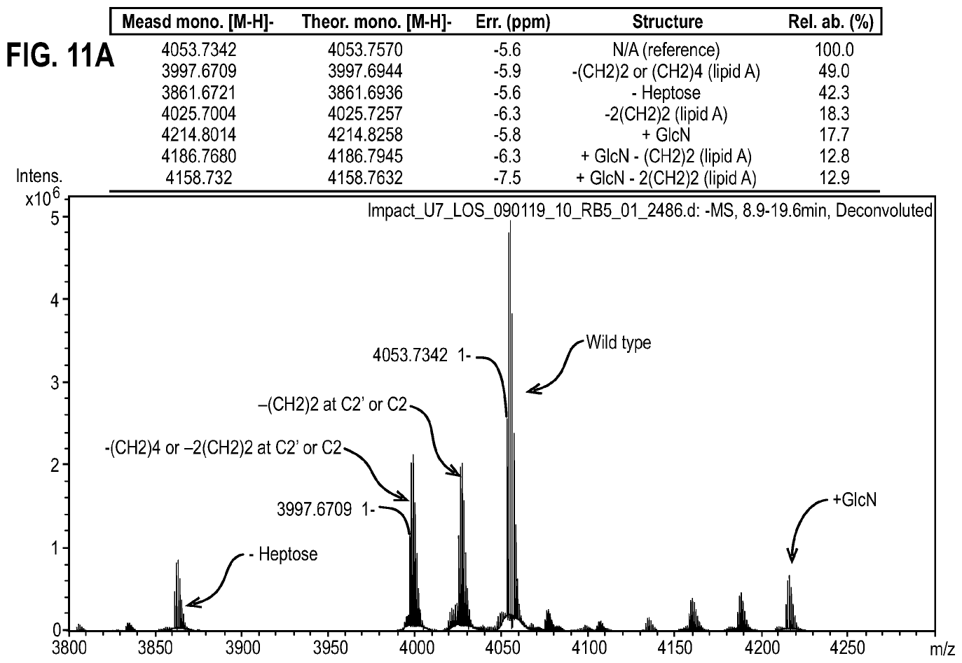

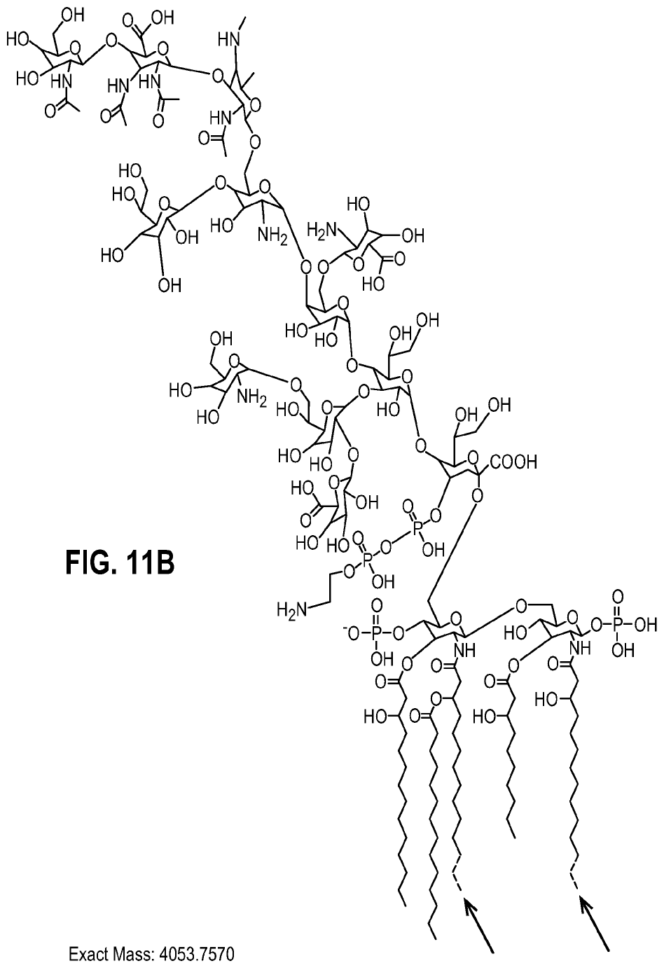

Episomal expression of LpxD from P. aeruginosa (LpxDPa) only had a partial impact on the LPS structure of the OMV from recombinant B. pertussis (FIG. 11), with just 36% of the total LPS displaying a reduced length at either both of the C2 and C2′ acyl chains (24%) or only one of the C2 or C2′ acyl chains (12%).

Genomic expression of LpxD_{Pa} increased the impact on LPS, wherein the length of both C2 and C2′ acyl chains was reduced in 70% of LPS, and the length of only one of C2 or C2′ was reduced in 30% of LPS (FIG. 9). Removal of the remaining activity of LpxD_{Bpe} led to 100% chain length reduction at both positions (ΔLpxA_{Bpe}/LpxA_{Bpa}/ΔDNT/LpxD_{Pa}/ΔLpxD_{Bpe}, FIG. 10).

□ArnT/PaqL Recombinant Strain

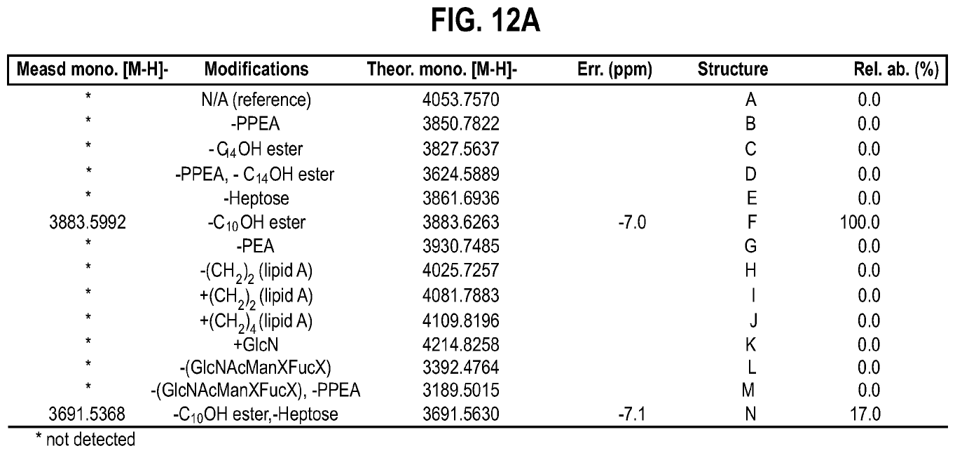

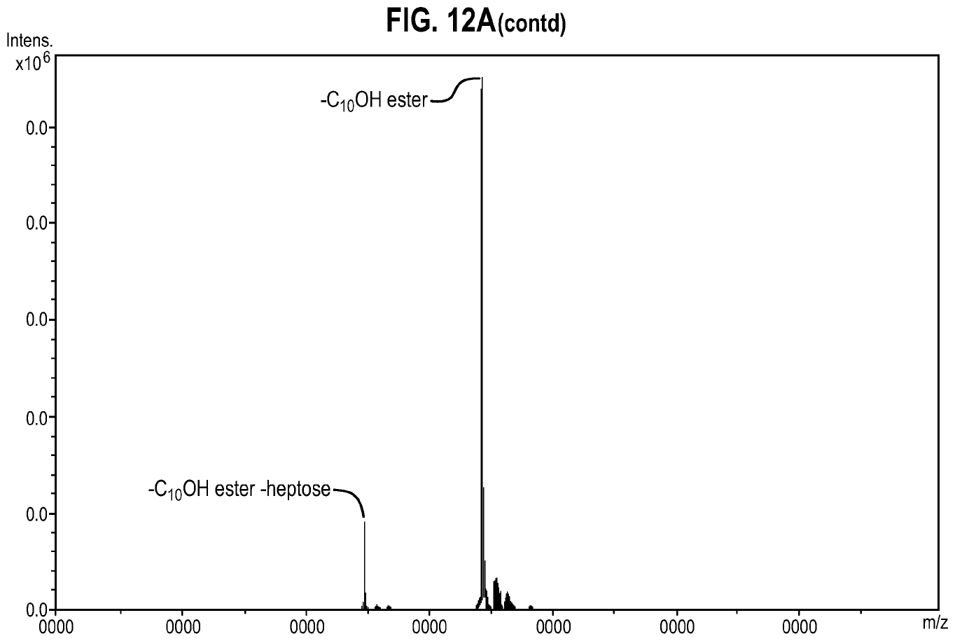

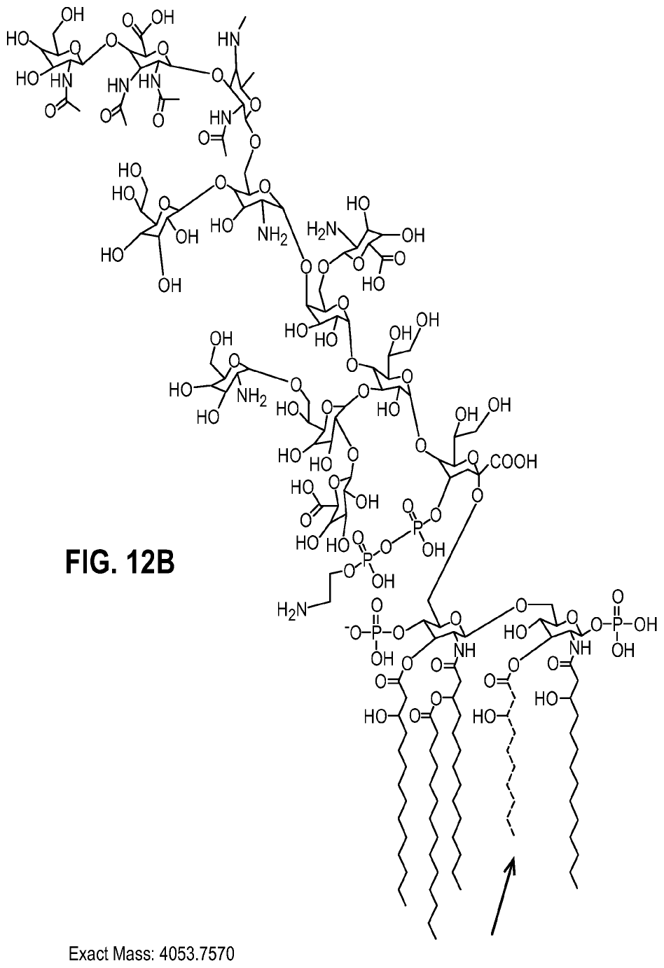

Inactivation of ArnT and genomic integration of PagL led to 100% removal of the C10 ester at C3 (FIG. 12). For reference, the structural analysis of OMVs from wild type B. pertussis Tomaha I Ptg is shown in FIG. 13.

LpxE Recombinant Strains

For LpxE from F. novicida (strain B2982, 9.8), LpxE_{Fn}, episomal expression resulted in 25% of monophosphoryl lipid A. Genomic expression of LpxE_{Fn} in the wild-type background increased the fraction of monophosphoryl lipid A to 91%. However, expression of the same construct in the LpxA mutant background surprisingly only produced 24% monophoshoryl lipid A. Similar results were observed with LpxE from R. leguminosarum (strain B2983, 9.9).

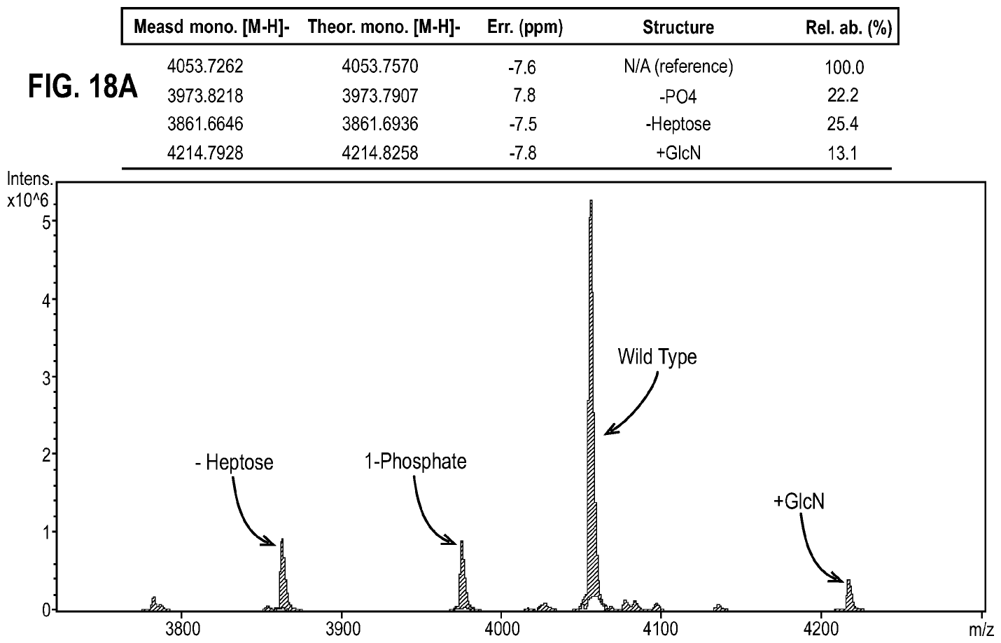

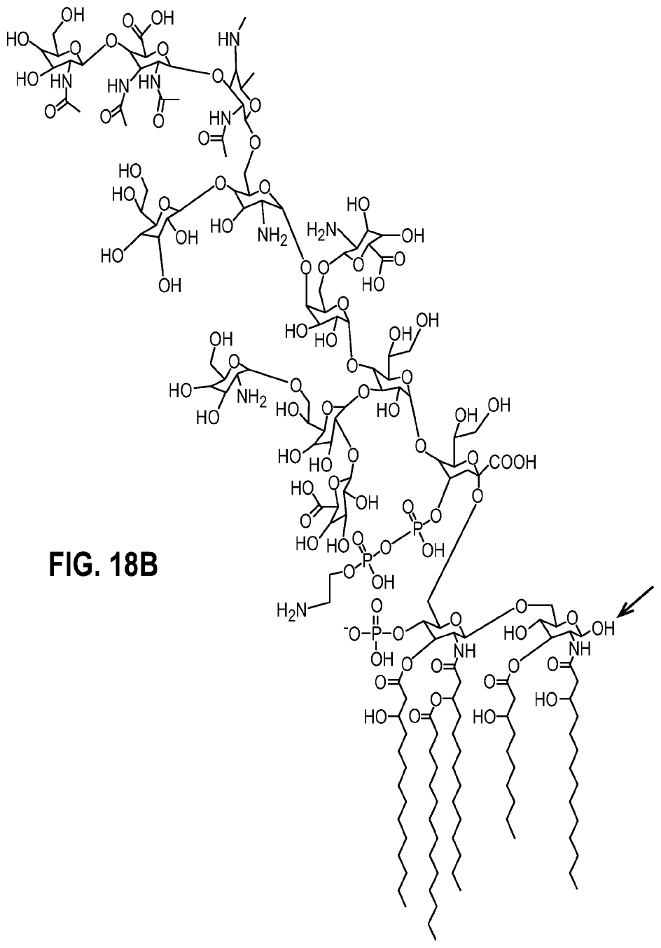

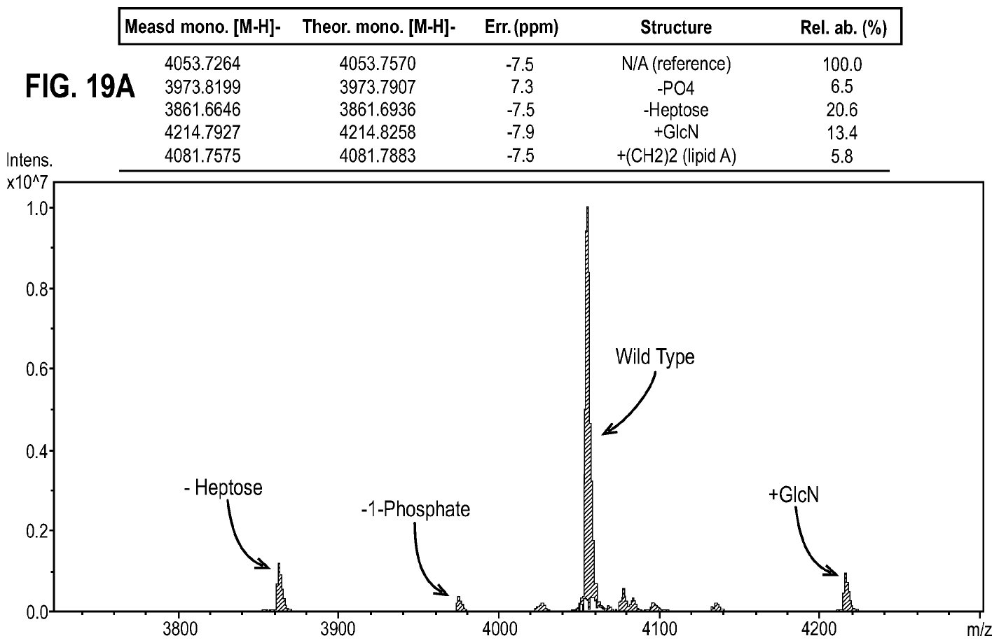

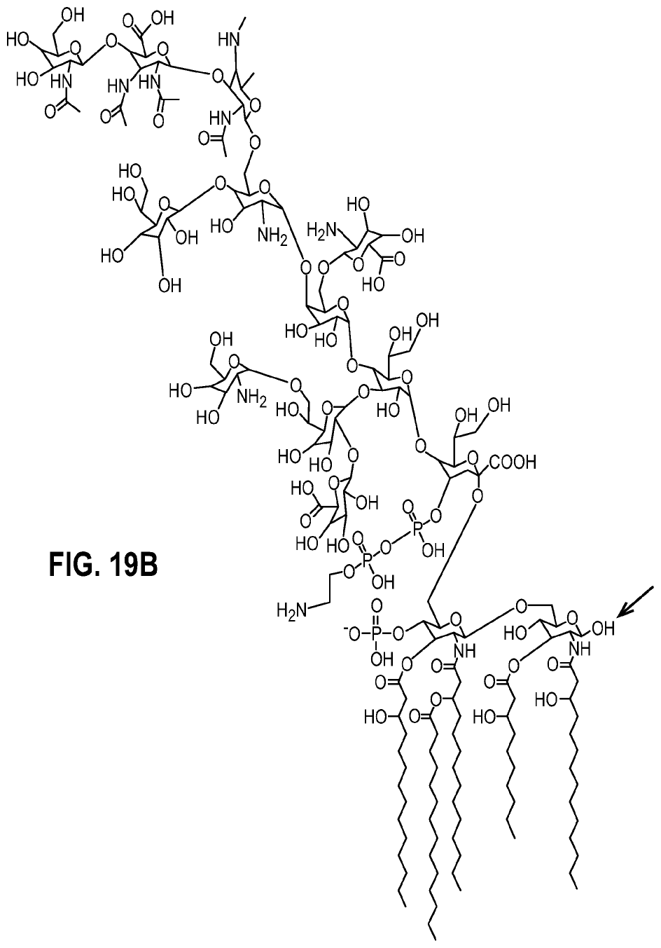

Structural data for the recombinant strains expressing LpxE_{F}n and LpxE_{R}c in the wild-type background is provided in FIGS. 18 and 19 (characterisation data for other combinations not specifically shown).

LpxF Recombinant Strains

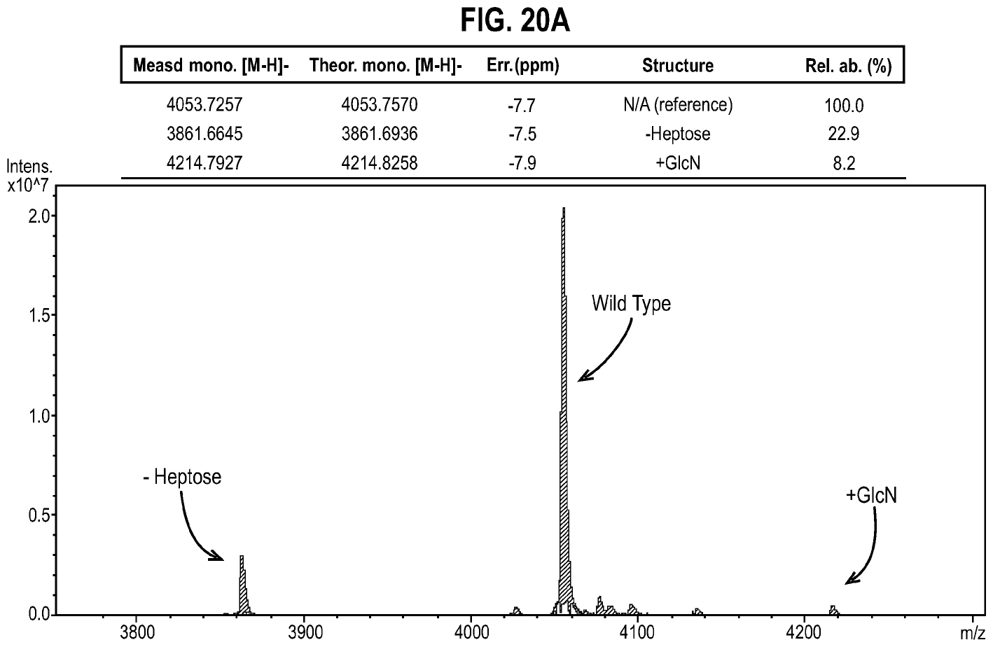

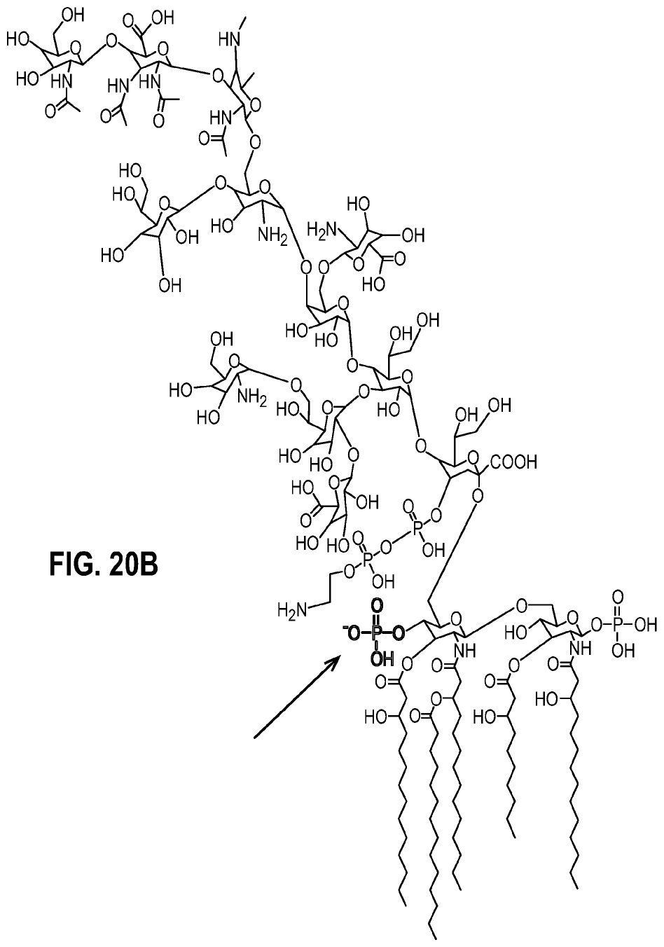

With regard to LpxF from F. novicida (strain B2981, 9.10), in a limited set of experiments, no identifiable impact on LOS structure was observed (FIG. 20).

In Vitro Evaluation of TLR4 Signalling Activation on HEK-hTLR4 Cells

Stimulation of the TLR4 signalling cascade can lead to the activation of NF-κB and/or activator protein 1 (AP-1) transcription factors, which contribute to the expression of several pro-inflammatory cytokines e.g. IL-1, IL-6, IL-8 and TNF-α and thus play a key role in inflammation.

To analyse whether the signalling activation of the TLR4 pathway decreased when using purified OMVs from recombinant B. pertussis, the TLR4 dependent transcriptional activity in HEK-Blue™-hTLR4 cells (Invivogen) was determined.

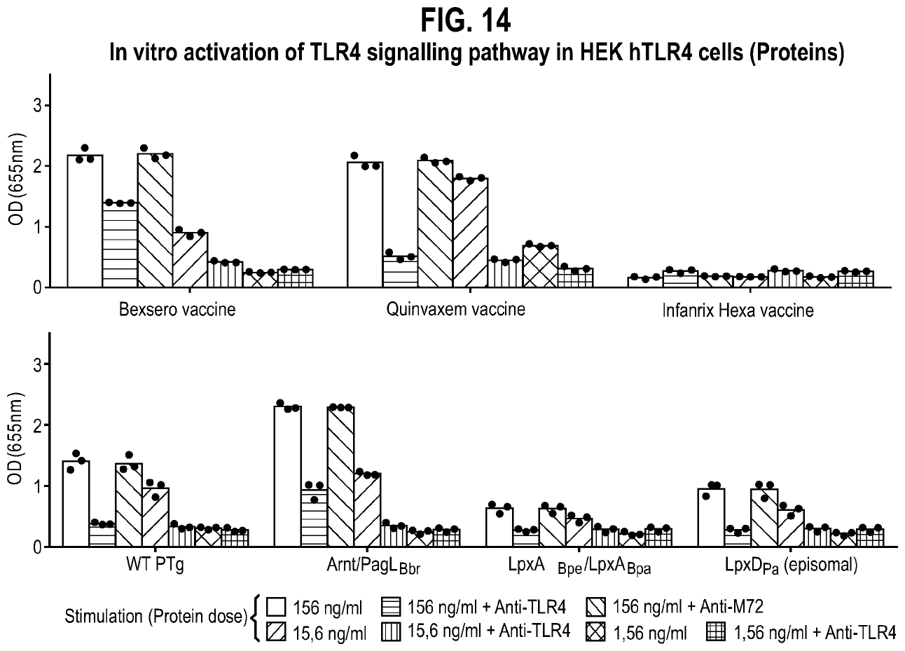

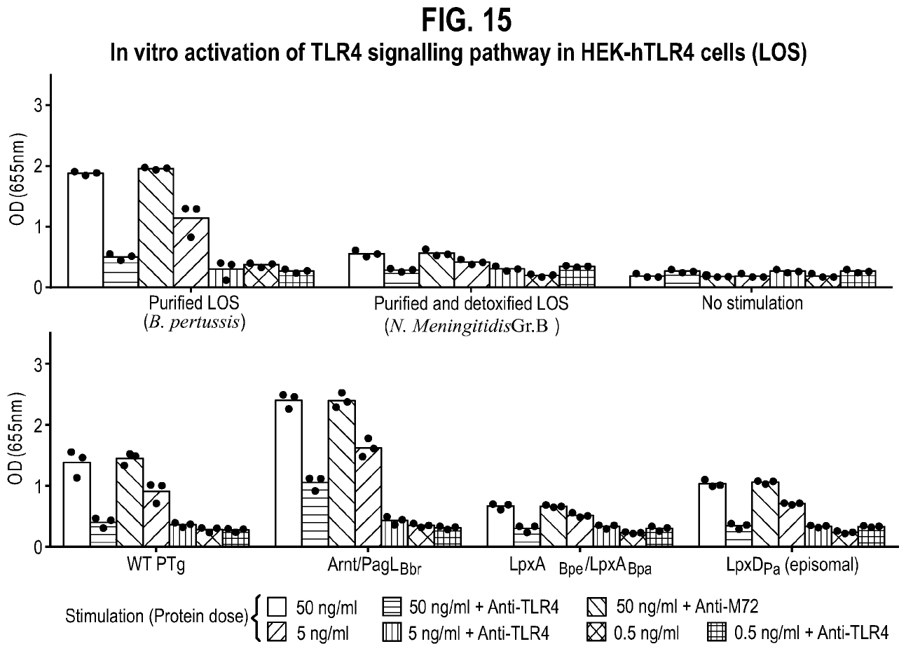

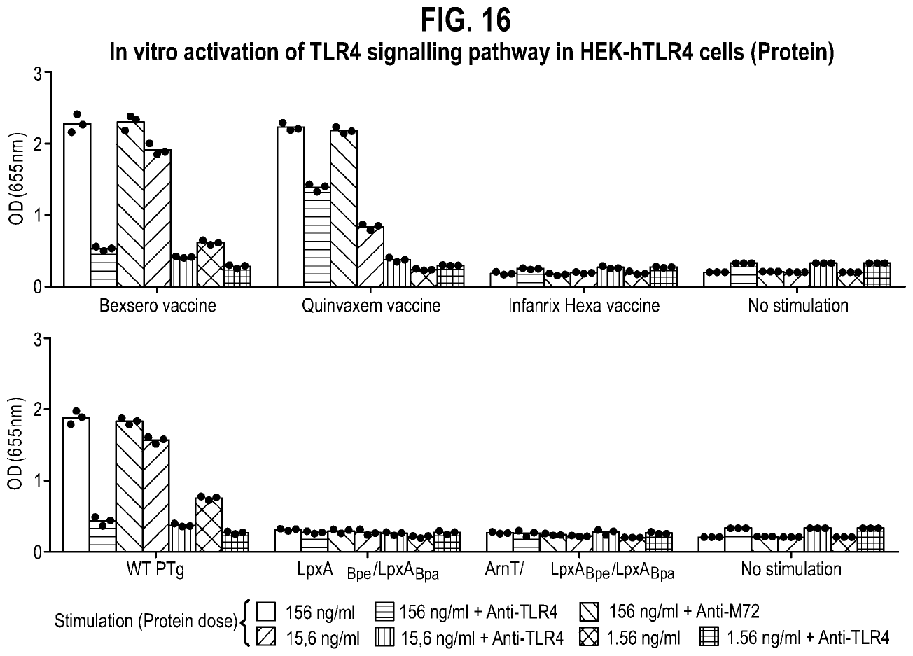

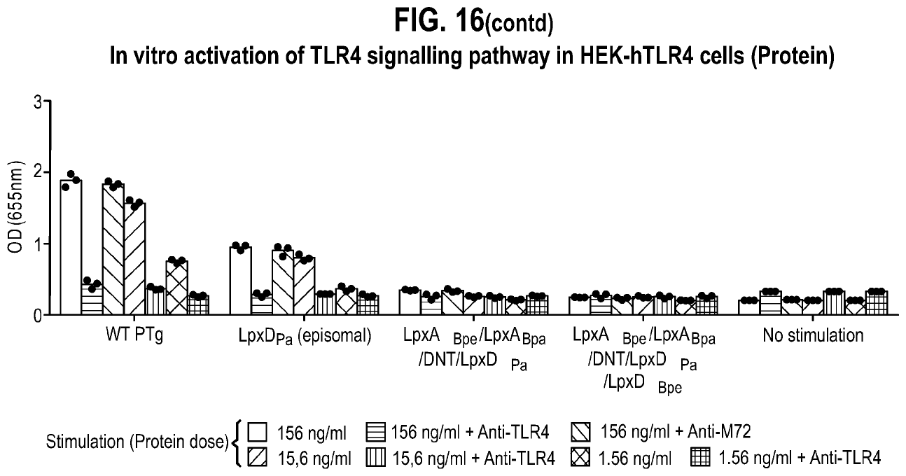

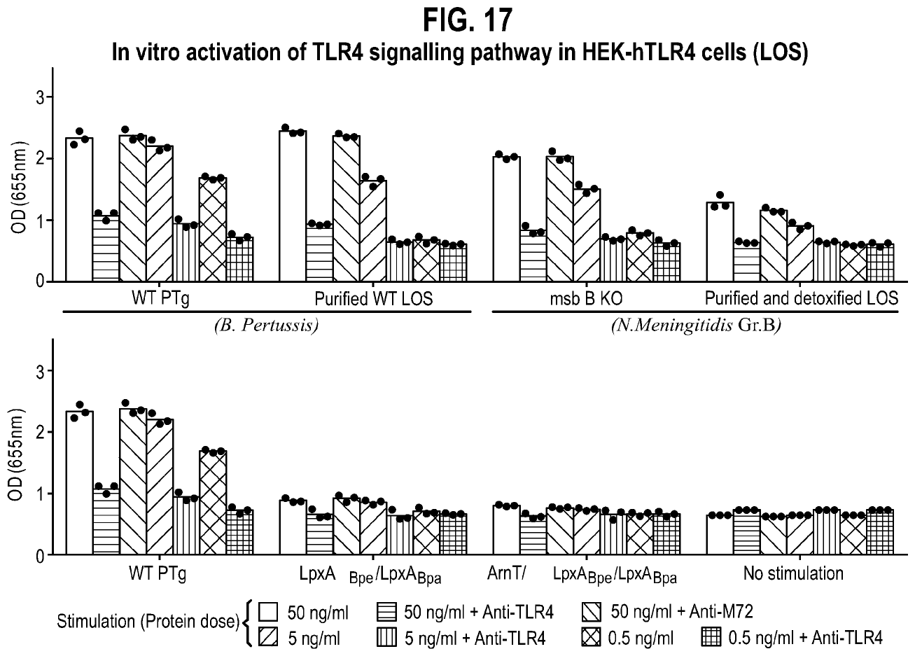

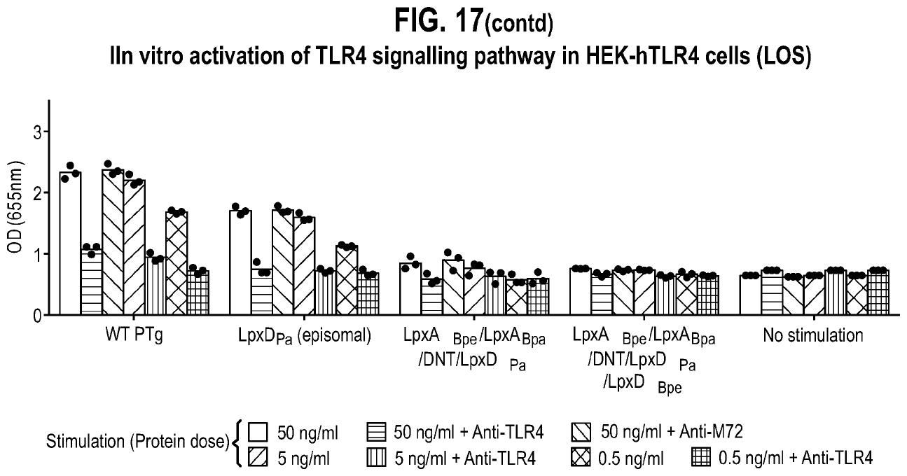

Upon stimulation with a TLR4 ligand, activation of NF-kB and AP-1 induces the secretion of alkaline phosphatase, which can be detected in the supernatant using the QUANTI-Blue™ colorimetric assay. TLR4 receptor response was determined measuring NF-κB- and AP-1-dependent SEAP production in supernatant measured at 655 nm. For each sample, three different dilutions were tested in triplicate. The proportion of signal attributable to TLR4 stimulation is visualized through specific blocking of the TLR4 receptor with an anti-TLR4 antibody, with an unrelated antibody (anti-M72) used as control. The capacity of the OMV obtained from the various recombinant B. pertussis strains to activate the TLR4 signalling was compared to that of the OMVs obtained from the Tohama I PTg strain (FIGS. 14 to 17).

For assays normalized by protein content, BEXSERO (MenB vaccine containing MenB OMV), QUINVAXEM (wP vaccine) and INFANRIX HEXA (aP vaccine) were included as controls. For assays normalized by LPS content, a purified LPS from B. pertussis, a detoxified (LpxL1 mutant) purified LPS from Neisseria and a negative control without stimulation were included.

The HEK-hTLR4 data showed a marked decrease in the activation of the TLR4 signalling cascade by OMVs from the LpxA standalone mutant ΔLpxA_{Bpe}/LpxA_{Bpa}. The activation profile was comparable (±0.5 OD) to that of INFANRIX HEXA i.e. low activation of the TLR4 signalling cascade.

Similar results were obtained with all the recombinant B. pertussis strains with genomic insertion of LpxA_{Bpa}, namely ΔArnT/ΔLpxA_{Bpe}/LpxA_{Bpa}, ΔLpxA_{Bpe}/LpxA_{Bpa}/ΔDNT/LpxD_{Pa}, and ΔLpxA_{Bpe}/LpxA_{Bpa}/ΔDNT/LpxD_{Pa}/ΔLpxD_{Bpe}.

In accordance with their partial effect on the LPS structure, those LpxD mutants in which LpxD was episomally expressed induced a more modest decrease in the TLR4 signalling cascade activation. More specifically, the OD₆₅₅ values observed with the LpxD mutants were higher (≥0.5 OD₆₅₅) than the OD₆₅₅ values observed in the non-stimulated cells and/or to the OD₆₅₅ values observed with cells stimulated with aP vaccine, but lower than the OD₆₅₅ values observed with the cells stimulated with OMV derived from wild type gram-negative bacterium. The reduction was observed in assays using both normalized protein and LPS.

The effect of the ΔArnT mutation in strain ΔArnT/ΔLpxA_{Bpe}/LpxA_{Bpa} cannot be easily derived since the decrease in TLR4 stimulation provided by the ΔLpxA_{Bpe}/LpxA_{Bpa} mutation already saturates the read-out and no further decrease can be observed. With OMV from strain ΔArnT/PagL_{Bbr} on the other hand, an increase in TLR4 stimulation is observed. Given such an increase is not observed in both ΔArnT mutant strains, this increase is hypothesized to be caused by the removal of the C3 acyl chain through PagL_{Bbr} activity.

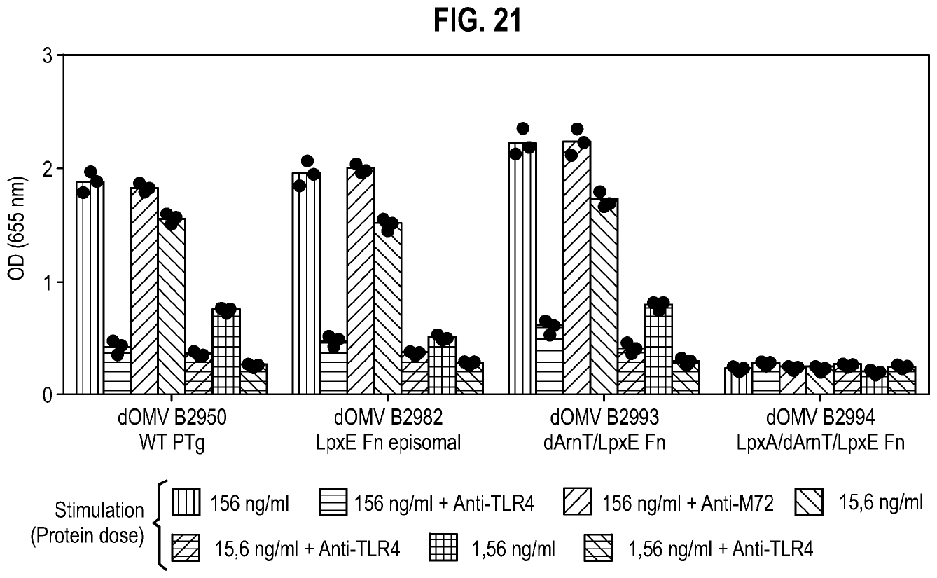

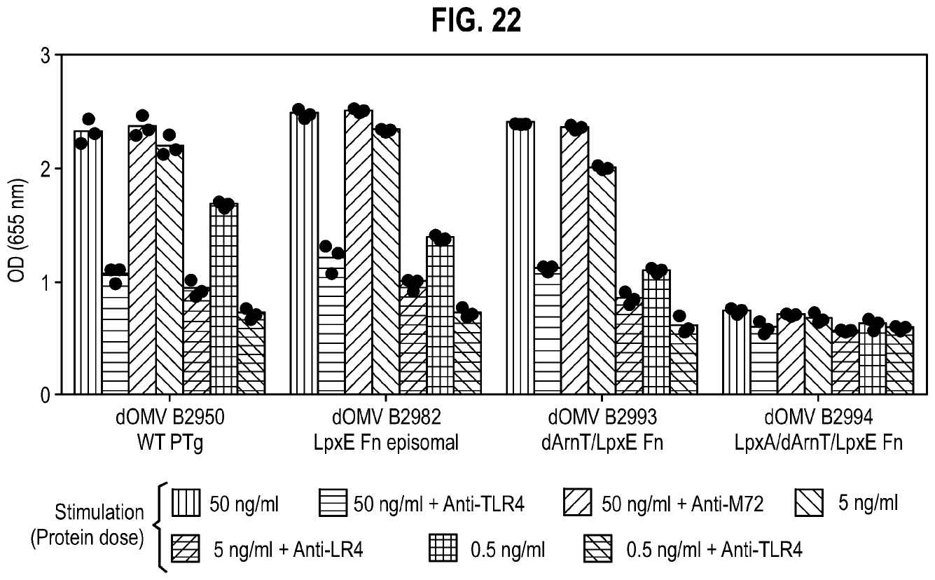

Interestingly, whilst the LpxE mutants altered the structure of lipid A, in the assays used, no significant decrease in TLR4 stimulation was detected (FIGS. 21 and 22).

Efficacy Study Conducted in the Nasopharynx Colonisation Model in Balb/c Mice

The goal of this study was to evaluate the impact of dOMV candidate vaccines in co-administration with DTPa in the nasopharynx (NP) colonization model in comparison with mice vaccinated with DTPa or DTPw vaccines, or with previously challenged mice (convalescent mice) against the wild-type Bordetella Pertussis Tohama I strain.

Experimental Design:

Twenty-five 6 weeks old female BALB/c mice were assigned to one of the groups listed in Tables 6 and 7 below. Table 6 details a first study involving 25 of the mice. All of these mice were previously challenged with 10⁶ CF/10 μl of the wild-type Bordetella Pertussis Tohama I strain (convalescent mice). This study is referred to as “LIMS 20190306”. The second study detailed in Table 7 and is referred to as “LIMS 20190307”.

Both studies were set-up in order to have the challenge on the same day with 10 CF/10 μl of the Bordetella Pertussis Tohama I strain. Therefore, convalescent mice (of LIMS 20190306) were older than vaccinated mice (of LIMS 20190307).

The distributions of mean of number of colony-forming unit (CFU) measured by counting the colony growth on Charcoal-céphalexin agar plates or by quantification by PCR are assumed to be lognormal. The statistical method is, by method (classical or by quantification), an Analysis of Variance (ANOVA) on the log 10 values with 2 factors (vaccine and day) and its interaction.

TABLE 6

| | | Treatment | | | | |
|---|---|---|---|---|---|---|
| | | | Adjuvant | | | |
| | | | dose- | | | |
| | No. | | Al(OH)3 | Immunization | | |
| Group | animals | Vaccine dose | (μg) | schedule | Challenge | Sample collection |
| 1 | 25 | Unvaccinated | | | Tohama | Bleed D29- |
| | | | | | Bp strain | D57-NACA |
| | | | | | D0 D58 | 2 h-4-7-14-21 |
| | | | | | | days after second |
| | | | | | | challenge |

TABLE 7

| | | Vaccines (given in separate sites) | | | | | | | |
|---|---|---|---|---|---|---|---|---|---|
| | | | Dose | | | | | | |
| | | | (Total | | | Volume | | | |
| | | | prot)/ | | | and route | | | |
| | No. of | | dose | | HD/dose | of | Immunization | | Sample |
| Group | animals | Site 1 | Al(OH)3 | Site 2 | Al | injection | schedule | Challenge | collection |
| 1 | 25 | Unvaccinated | — | Unvaccinated | — | — | — | Tohama | Bleed D28- |
| 2 | 25 | — | — | Infanrix ™ | ¼th/ | 1 × 125 μl | D0-21 | Bp strain | NACA 2 h- |
| | | | | AC14B249C1 | 125 μg | (SC) | | D29 | 4-7-14-21 |
| 3 | 25 | — | — | Easy-Five ™ | ¼th/ | 1 × 125 μl | D0-21 | | days after |
| | | | | E5PFS8016 | 62.5 μg | (SC) | | | challenge |
| 4 | 25 | OMV WT- | 25 μg/ | Infanrix ™ | ¼th/ | 2 × 125 μl | D0-21 | | |
| | | (OMV034)/ | 125 μg | AC14B249C1 | 125 μg | (SC) | | | |
| | | Al(OH)3 | | | | | | | |
| 5 | 25 | OMV LpxA- | 25 μg/ | Infanrix ™ | ¼th/ | 2 × 125 μl | D0-21 | | |
| | | (OMV035)/ | 125 μg | AC14B249C1 | 125 μg | (SC) | | | |
| | | Al(OH)3 | | | | | | | |

Results:

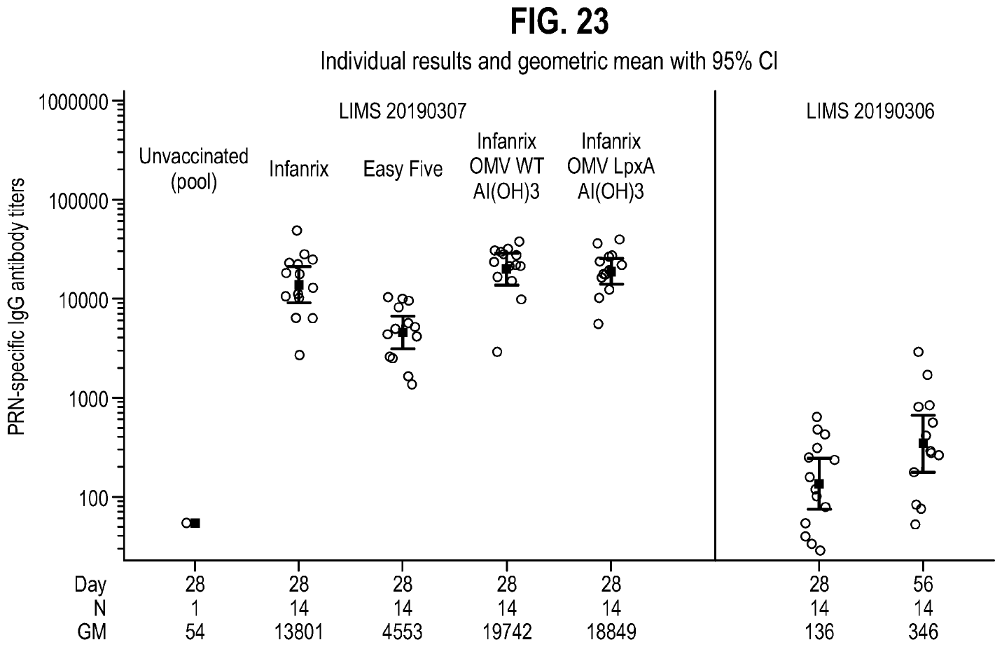

Humoral immune responses are shown in FIG. 23. FIG. 23 presents individual antibody titers anti-PPRN IgG measured by ELISA at day 28 as well as geometric mean titers (GMTs) derived from the ANOVA model and their 95% confidence intervals. Note that group in study 20190306 has been evaluated at days 28 and 56.

For the anti-PRN IgG measured by ELISA at day 28, immunogenicity is observed in all vaccinated groups (Infanrix, Easy Five, OMV LPXA and OMV WT). However, lower immunogenicity (GMR >3) is observed after Easy Five vaccination as compared to the other vaccinated groups. More, large lower immunogenicity (GMT of 136 and 346 respectively) is shown for convalescent mice as compared to vaccinated groups at the 2 timepoint 028 and D56.

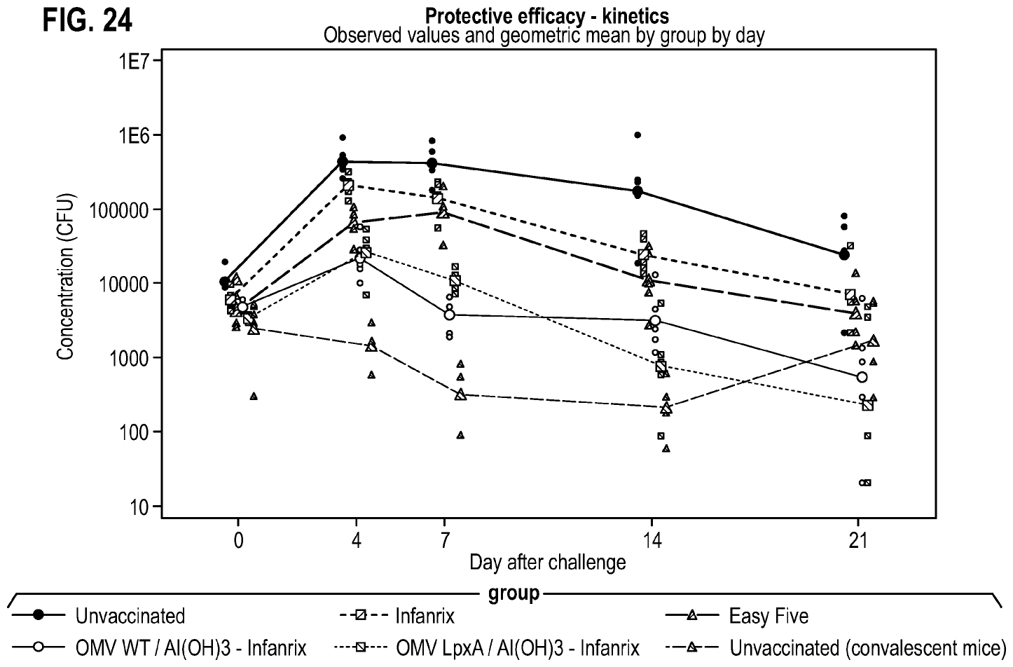

Evaluation of protection concerns bacterial load as defined by the mean of number of colony-forming unit (CFU) per nasal cavity for each collection time (+2h and days 4, 7, 13, 21 after challenge). Descriptive statistics are presented in FIG. 24.

A significant difference of bacterial load was highlighted between the no vaccine group (i.e. the unvaccinated group 1 of Table 7) and all other groups, suggesting some level of protective immune response against nasal colonization after vaccination or re-infection.

More than 10 log CFU reduction (GMR=258) was observed in convalescent animals (of Table 6) as compared to the unvaccinated animal, which confirms the suitability of this group as a positive control.

Altogether this suggests that this model allows detection of reduced nasal colonization.

More than 1 log CFU reduction was observed with OMV LpxA-Infanrix (GMR=61) (group 5 of Table 7) and OMV WT-Infanrix (GMR=48) (group 4 of Table 7) as compared to unvaccinated animals, and as compared to Infanrix alone (GMR=17.7 & 13.8 respectively) but not as compared to the unvaccinated (convalescent mice) group (GMR=4.2 and GMR=5.4 respectively).

Vaccination with both OMV vaccines in co-administration with DTPa similarly reduced the nasal colonization following a challenge with Bordetella Pertussis Tohama I strain as compared to both unvaccinated and Infanrix (DTPa) vaccinated mice. This indicates an added value of OMV on prevention of nasopharynx colonization as compared to DTPa vaccine. This also indicates that detoxification of Lipid A did not negatively impact the reduction of nasal colonization.

Efficacy Study Conducted in the Lung Clearance Model in Balb/c Mice

The primary goal of this study was to evaluate the added value of dOMVvaccines in co-administration with DTPa on protective efficacy mice model against the Bordetella Pertussis PRN(−) strain.

The protective efficacy was evaluated by determining the bacterial load per lung for each collection time (+2h and days 2, 5 and 8 after challenge) by the mean of number of colony-forming unit (CFU−log 10).

Experimental Design:

Twenty 6 weeks old female BALB/c were randomly assigned to one of the study groups detailed in Table 8. The mice were challenged on day 29 with 5×10⁶ CFU/50 μl of the PRN(−) Bordetella Pertussis strain FR5388.

TABLE 8

| | | Vaccines (given in separate sites) | | | | | | | |
|---|---|---|---|---|---|---|---|---|---|
| | | | Dose | | | | | | |
| | | | (Total | | | Volume | | | |
| | Number | | prot)/ | | | and | | | |
| | of | | dose | | HD/dose | route of | Immunization | | Sample |
| Group | animals | Site 1 | Al(OH)3 | Site 2 | Al | injection | schedule | Challenge | collection |
| 1 | 20 | Unvaccinated | — | Unvaccinated | — | — | — | PRN(—) | Bleed |
| 2 | 20 | — | — | Infanrix ™ | ¼th/ | 1 × 125 μl | D0-21 | Bp strain | D28- |
| | | | | | 125 μg | (SC) | | FR5388 | Lung 2 h- |
| | | | | | | | | D29 | 2-5-8 |
| 3 | 20 | — | — | Easy-Five ™ | ¼th/ | 1 × 125 μl | D0-21 | | days after |
| | | | | | 62.5 μg | (SC) | | | challenge |
| 4 | 20 | OMV WT/ | 25 μg/ | Infanrix ™ | ¼th/ | 2 × 125 μl | D0-21 | | Bleed |
| | | Al(OH)3 | 125 μg | | 125 μg | (SC) | | | D28 |
| 5 | 20 | OMV LpxA/ | 25 μg/ | Infanrix ™ | ¼th/ | 2 × 125 μl | D0-21 | | |
| | | Al(OH)3 | 125 μg | | 125 μg | (SC) | | | |
| 6 | 20 | OMV | 25 μg/ | Infanrix ™ | ¼th/ | 2 × 125 μl | D0-21 | | |
| | | LpxA/LpxD/ | 125 μg | | 125 μg | (SC) | | | |
| | | Al(OH)3 | | | | | | | |

Results:

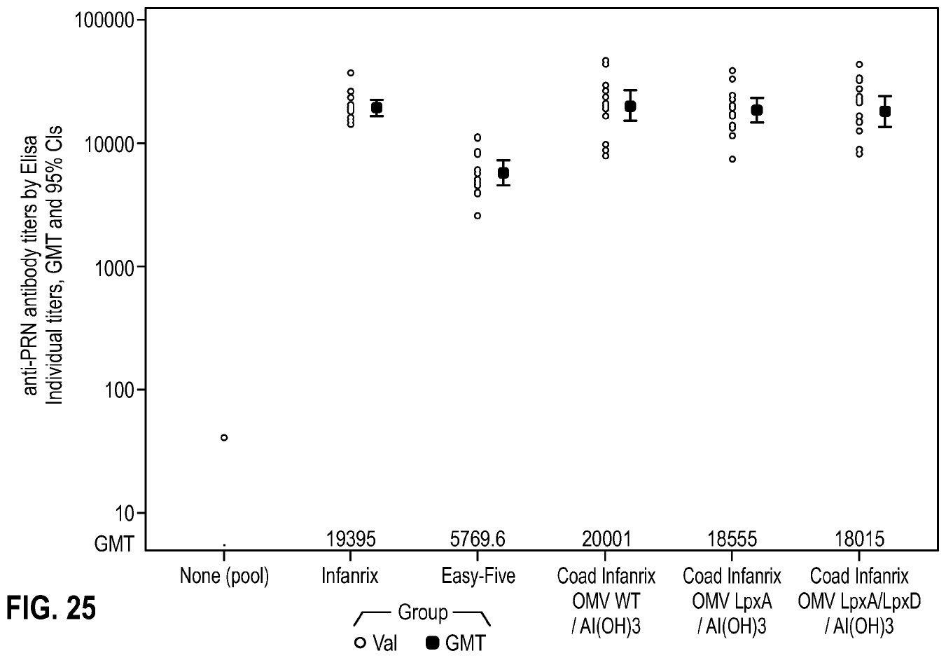

Humoral immune responses are shown in FIG. 25. FIG. 25 presents individual anti-PRN IgG titers measured by ELISA at day 28 (1 day before challenge) as well as geometric mean titers (GMTs) derived from the ANOVA model and their 95% confidence intervals. Vaccine take was confirmed as seen by anti-PRN IgG in all vaccinated groups in FIG. 25. All groups induced similar anti-PRN response except for “Easy-Five” group which has an estimated GMT about 3-fold lower than the other groups. These differences have been detected as statistically significant.

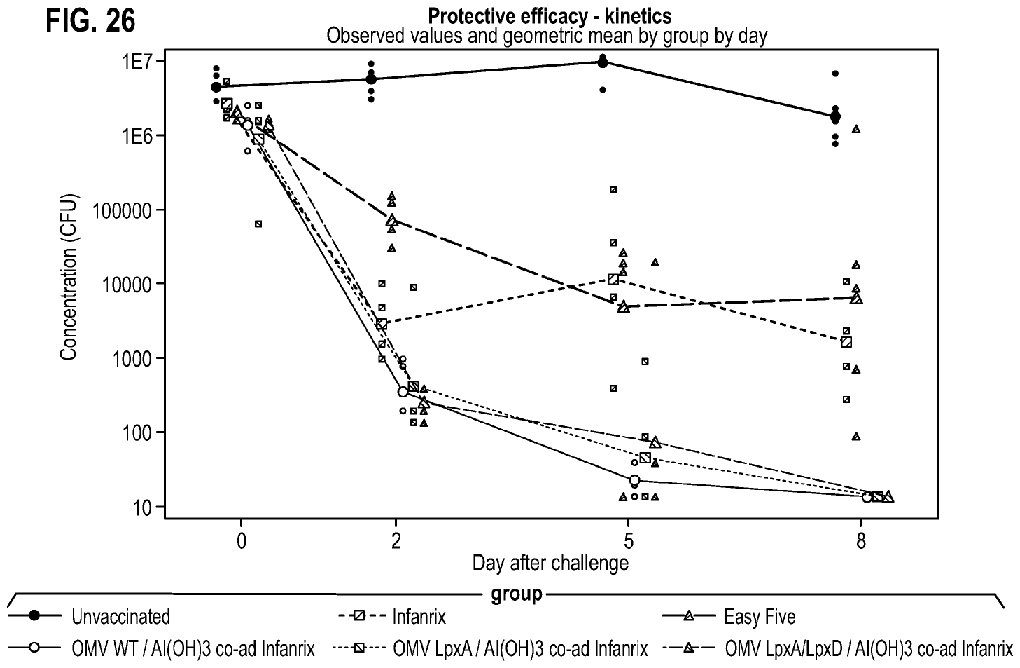

Evaluation of the protection against Pertussis concerns bacterial load as defined by the mean of number of colony-forming unit (CFU) per lung for each collection time (+2h and days 2, 5, 8 after challenge). Descriptive statistics are presented in FIG. 26. As shown in FIG. 26, a significant difference was highlighted between the no vaccine group and the other groups suggesting a protective immune response after vaccination with a Pertussis containing vaccine. Partial protection was observed in group immunized with Infanrix or Easy Five. A significant difference was observed between group immunized with Infanrix and group that also received dOMV in co-administration, indicating an added value of dOMV on top of DTPa vaccine in lung clearance of PRNneg strain.

It was expected to obtain different kinetic profiles depending on the group (e.g. Infanrix and Easy five should be different to all the other groups) and this is reflected by a significant interaction between group and day in the statistical analysis.

Higher CFU reductions are observed following vaccination with OMV (GMR between 130000 and 42000 at day 5 and equal to 127439 at day 8) as compared to unvaccinated group and (GMR less than 2000 at days 5 and 8) as compared to Infanrix or Easy five groups. No difference is observed between these 3 OMV groups. These results are positive as they show that the OMV LpxA and the OMV LpxA/LpxD are as effective as the OMV WT at reducing clearing lung from B. pertussis PRN negative strain colonization.

Stimulation of Human PBCs

Experimental Design:

An experiment was performed to evaluate the reactogenicity biomarker profile (innate cytokines) secretion by stimulated human peripheral blood mononuclear cells (PBMCs). Frozen PBMCs collected from 3 healthy blood donors were thawed and individually seeded in 96-well plate at a concentration of 5E6 cells per ml. The cells were then incubated for 24 hours at 37° C. with protein-equivalent formulations. Eight serial dilutions, with a step of 5-fold between them were prepared as detailed in Table 9. At the end of the incubation, cytokines were measured using multiplex assay in harvested culture supernatants.

Human standard and controls were prepared for the testing. In this test, the following cytokines were tested: IL-6, IL-10, TNF-α (“TFNa”), IL-1α (“IL-1a”), IL-1β (“IL-1b”) and MIP-1 α (“MIP1a”).

TABLE 9
Stimulation conditions

| | | Final | Final |
|---|---|---|---|
| | Stock protein | concentration | concentration |
| | Concentration | of dose #1 | of dose #8 |
| | (μg/ml) | (μg/ml) | (μg/ml) |
| Bexsero | 50 | 1.25 | 0.000016 |
| Quinvaxem | 50 | 1.25 | 0.000016 |
| Infanrix Hexa | 50 | 1.25 | 0.000016 |
| Vaxelis | 50 | 1.25 | 0.000016 |
| Easy Five | 50 | 1.25 | 0.000016 |
| dOMV WT non | 730 | 1.25 | 0.000016 |
| adsorbed (PTg WT) | | | |
| dOMV WT adsorbed | 200 | 1.25 | 0.000016 |
| on Al(OH)3 (PTg WT) | | | |
| dOMV LpxA non | 310 | 1.25 | 0.000016 |
| adsorbed | | | |
| dOMV LpxA adsorbed | 200 | 1.25 | 0.000016 |
| on Al(OH)3 | | | |
| dOMV LpxA/LpxD | 870 | 1.25 | 0.000016 |
| non adsorbed | | | |
| dOMV LpxA/LpxD | 200 | 1.25 | 0.000016 |
| adsorbed on Al(OH)3 | | | |

Results:

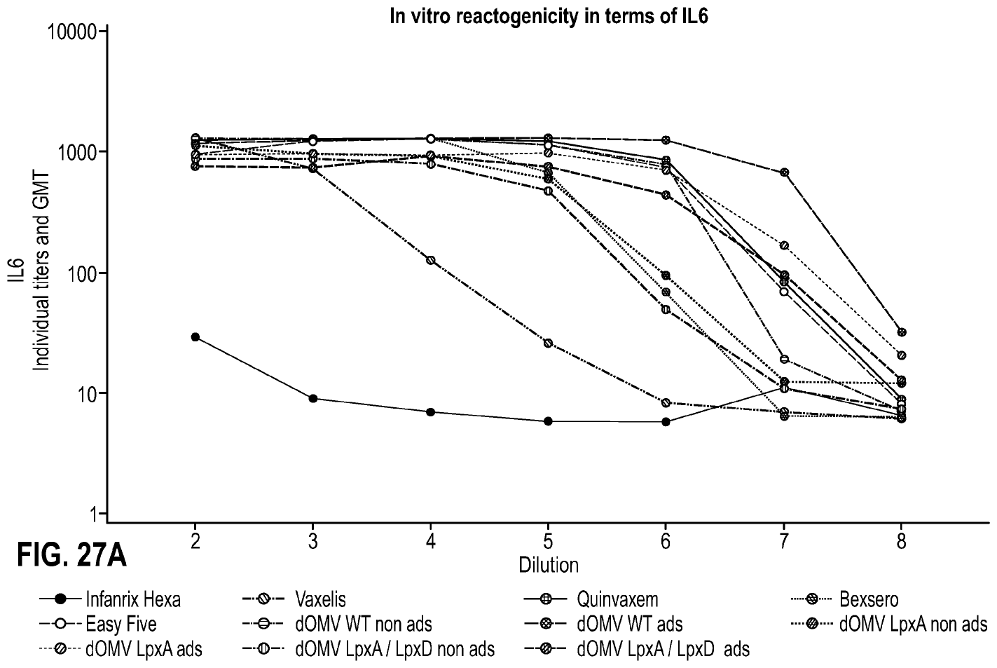

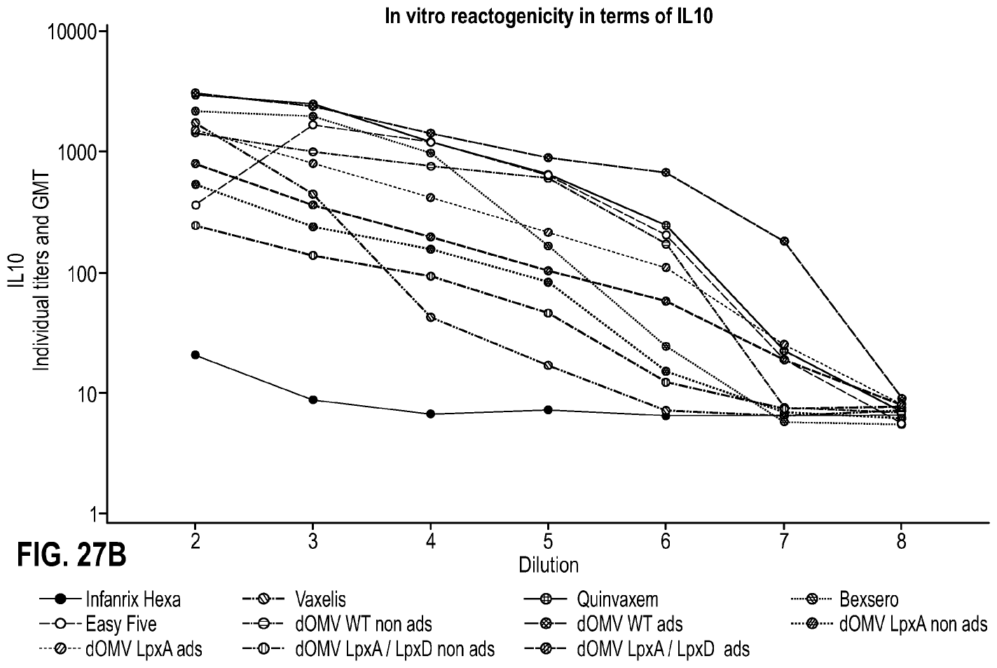

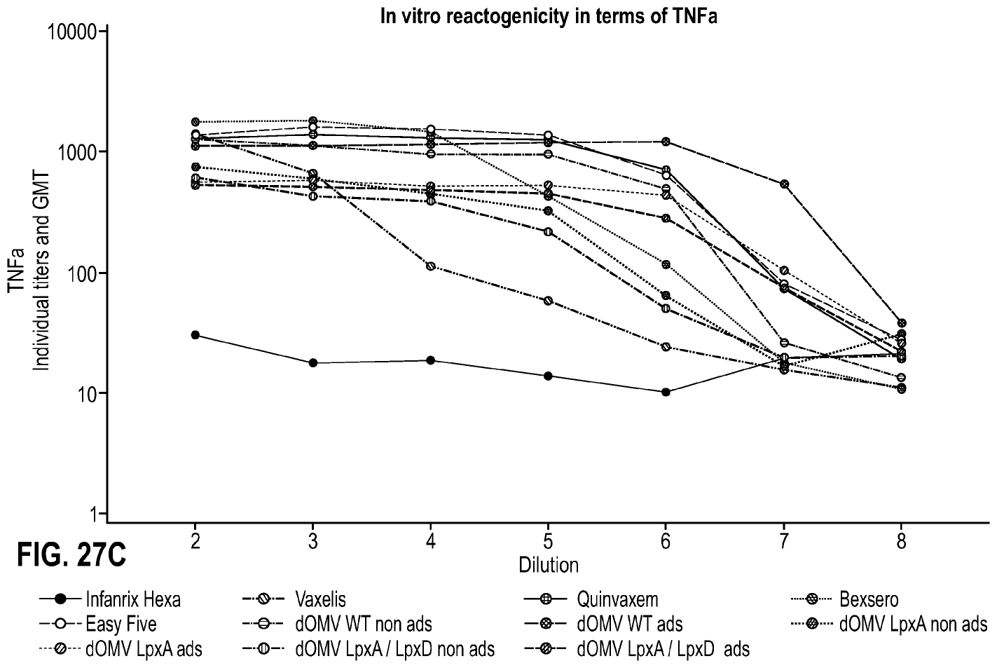

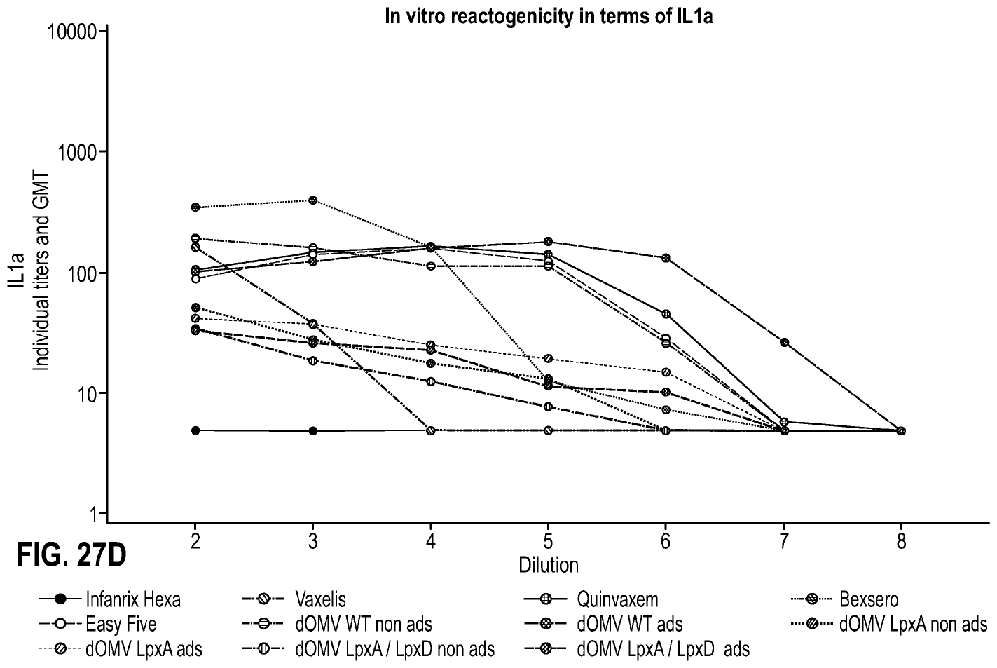

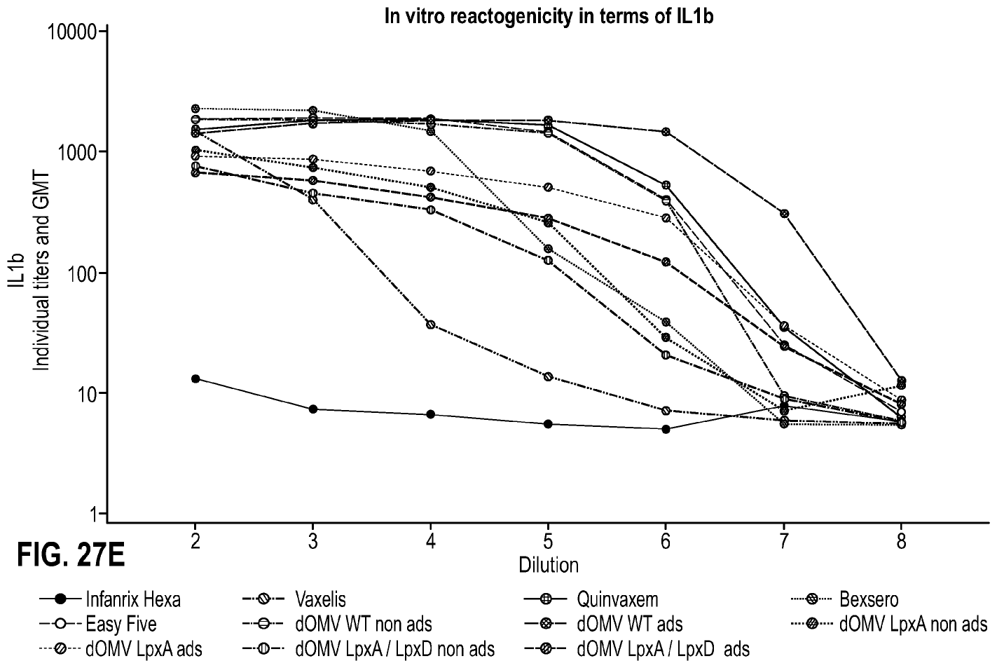

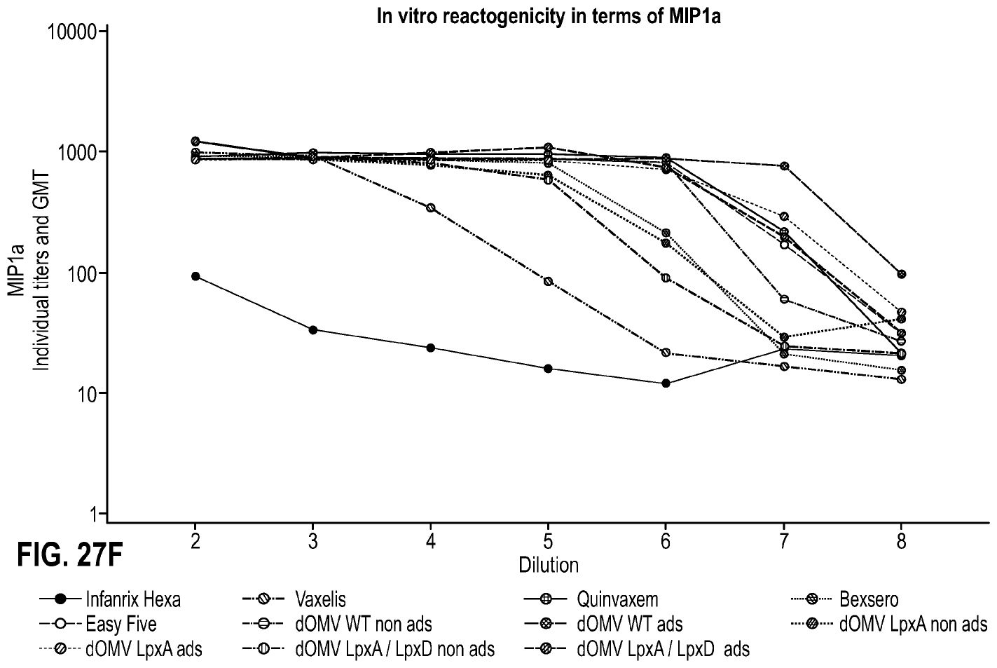

Six cytokines (i.e.: IL-6, IL-10, TNF-α (“TFNa”), IL-1α (“IL-1a”), IL-1β (“IL-1b”) and MIP-1 α (“MIP1a”)) known to be associated with reactogenicity mechanisms were measured in culture supernatants for the different serial dilutions. FIG. 27 corresponds to the mean of the 3 donors by stimulation and by cytokine (excluding dilution #1, which altered cell viability in these experimental conditions).

FIG. 27 shows that OMV LpxA showed reduced innate cytokine secretion by human PBMCs as compared to OMV wild type (WT), suggesting a reduced reactogenicity of OMV harboring the LpxA mutation.

From Kruskal-Wallis Area under the curve (AUC) analysis grouping cytokines and dilutions (but still excluding dilution #1) on all dilutions (see FIG. 27 and Table 9), it was observed that the dOMV WT adsorbed on Al(OH)₃ was the only condition that induced a statistically significantly higher innate cytokines than Infanrix Hexa. No statistical differences was observed between the other conditions. Non-adsorbed dOMV displaying mutation on LpxA or LpxA/LpxD showed a trend of reduced innate cytokine secretion by human PBMCs as compared to OMV WT, suggesting a reduced reactogenicity of OMV harboring the LpxA or LpxA/D mutations. The level of innate cytokine secretion induced by OMV LpxA was in the same range as Bexsero and tended to be lower than a whole cell vaccine.

It will be understood that the invention has been described by way of example only and modifications may be made whilst remaining within the scope and spirit of the invention as defined in the appended claims.

### REFERENCES

[1] Acevedo R. et al., Bacterial outer membrane vesicles and vaccine applications. Front. Immunol. 2014 Mar. 24; 5:121.
[2] Maeshima N., Fernandez R. C. (2013) Recognition of lipid A variants by the TLR4/MD-2 receptor complex. Frontiers in Cellular and Infection Microbiology 3:3.
[3] WO2018/167061
[4] Sweet C. R., Preston A., Toland E., Ramirez S. M., Cotter R. J., Maskell D. J., Raetz C. R. (2002) Relaxed acyl chain specificity of Bordetella UDP-N-acetylglucosamine acyltransferases. Journal of Biological Chemistry 277(21):18281-90.
[5] Bartling C. M., Raetz C. R. (2009) Crystal structure and acyl chain selectivity of Escherichia coli LpxD, the N-acyltransferase of lipid A biosynthesis. Biochemistry 48(36):8672-83
[6] Steeghs L, Berns M, ten Hove J, de Jong A, Roholl P, van Alphen L, Tommassen J, van der Ley P. (2002) Expression of foreign LpxA acyltransferases in Neisseria meningitidis results in modified lipid A with reduced toxicity and retained adjuvant activity. Cell Microbiol. 2002 September; 4(9):599-611.
[7] EP0011243
[8] Fredriksen et al. (1991) Production, characterization and control of MenB-vaccine “Folkehelsa”: an outer membrane vesicle vaccine against group B meningococcal disease. NIPH Ann. 14(2):67-80
[9] WO2004/019977
[10] U.S. Pat. No. 6,558,677
[11] WO2005/004908
[12] WO2011/036562
[13] Gennaro (2000) Remington: The Science and Practice of Pharmacy. 20th edition, ISBN: 0683306472.
[14] Vaccine Design (1995) eds. Powell & Newman. ISBN: 030644867X. Plenum.
[15] WO90/14837.
[16] Module 1 of WHO's The immunological basis for immunization series (Galazka)
[17] Lyng (1990) Biologicals 18:11-17
[18] NIBSC code: 69/017
[19] Kuhmlann & Rieger (1995) Immunol Infect Dis 5:10-4
[20] Sesardic et al. (2002) Biologicals 30:49-68
[21] NIBSC code: 98/552
[22] Lindberg (1999) Vaccine 17 Suppl 2:S28-36
[23] Buttery & Moxon (2000) J R Coll Physicians Lond 34:163-168
[24] Ahmad & Chapnick (1999) Infect Dis Clin North Am 13:113-133, vii
[25] Goldblatt (1998) J. Med. Microbiol. 47:563-567
[26] EP-A-0477508
[27] U.S. Pat. No. 5,306,492
[28] WO1998/42721
[29] Conjugate Vaccines (eds. Cruse et al.) ISBN 3805549326, particularly vol. 10:48-114
[30] Lees et al. (1996) Vaccine 14:190-198
[31] WO1995/08348
[32] WO1998/42721
[33] U.S. Pat. No. 4,882,317
[34] U.S. Pat. No. 4,695,624
[35] EP-B-0477508
[36] Mol. Immunol. 1985, 22, 907-919
[37] EP-A-0208375
[38] WO2000/10599
[39] Gever et al. (1979) Med. Microbiol. Immunol. 165:171-288
[40] U.S. Pat. No. 4,057,685
[41] U.S. Pat. No. 4,673,574
[42] U.S. Pat. No. 4,761,283;
[43] U.S. Pat. No. 4,808,700
[44] U.S. Pat. No. 4,459,286
[45] U.S. Pat. No. 4,965,338
[46] U.S. Pat. No. 4,663,160
[47] Research Disclosure, 453077 (January 2002)
[48] Anderson (1983) Infect Immun 39(1):233-238
[49] Anderson et al. (1985) J Clin Invest 76(1):52-59
[50] Kanra et al. (1999) The Turkish Journal of Paediatrics 42:421-427
[51] Ravenscroft et al. (2000) Dev Biol (Basel) 103: 35-47
[52] WO96/40242
[53] Verez-Bencomo V, Fernández-Santana V, Hardy E, Toledo M E, Rodriguez M C, Heynngnezz L, Rodriguez A, Baly A, Herrera L, Izquierdo M, Villar A, Valdés Y, Cosme K, Deler M L, Montane M, Garcia E, Ramos A, Aguilar A, Medina E, Torano G, Sosa I, Hernandez I, Martinez R, Muzachio A, Carmenates A, Costa L, Cardoso F, Campa C, Diaz M, Roy R. A synthetic conjugate polysaccharide vaccine against Haemophilus influenzae type b. Science. 2004 Jul. 23; 305(5683):522-5.
[54] WO2018/020046
[55] Vanlandschoot et al. (2005) J Gen Virol 86:323-31
[56] WO2007/054820
[57] WO2003/066094
[58] Module 6 of WHO's The immunological basis for immunization series (Robertson)
[59] WO2008/028956
[60] WO2008/028957
[61] Liao et al. (2012) J Infect Dis. 205:237-43
[62] Stibitz S, Black W, Falkow S. The construction of a cloning vector designed for gene replacement in Bordetella pertussis. Gene. 1986; 50(1-3):133-140.
[63] Goffin P, Slock T, Smessaert V, De Rop P, Dehottay P. A versatile, non genetically modified organism (GMO)-based strategy for controlling low-producer mutants in Bordetella pertussis cultures using antigenic modulation. Biotechnol J. 2015 August; 10(8):1269-80.
[65] Karbarz M. J., Kalb S. R., Cotter R. J., Raetz C. R. (2003) Expression cloning and biochemical characterization of a Rhizobium leguminosarum lipid A 1-phosphatase. Journal of Biological Chemistry 278(41):39269-79.
[66] Wang X., Karbarz M. J., McGrath S. C., Cotter R. J., Raetz C. R. (2004) MsbA transporter-dependent lipid A 1-dephosphorylation on the periplasmic surface of the inner membrane: topography of Francisella novicida LpxE expressed in Escherichia coli. Journal of Biological
[67] Wang X., McGrath S. C., Cotter R. J., Raetz C. R. (2006) Expression cloning and periplasmic orientation of the Francisella novicida lipid A 4′-phosphatase LpxF. Journal of Biological Chemistry 281(14):9321-30.
[68] WO2013/130779
[69] WO2015/179270

1. A recombinant Bordetella pertussis bacterium which comprises:
(i) at least one genomic LpxA gene encoding an LpxA protein, wherein the LpxA protein comprises a mutation at position 170 and/or a mutation at position 229 relative to SEQ ID NO: 1; and/or
(ii) at least one genomic insertion of a heterologous LpxD gene.

2. The recombinant Bordetella pertussis bacterium of claim 1 which comprises at least one genomic LpxA gene encoding an LpxA protein, wherein the LpxA protein comprises a substitution at position 170 and/or a substitution at position 229 relative to SEQ ID NO: 1.

3. The recombinant Bordetella pertussis bacterium of claim 1 wherein the LpxA protein comprises a Serine residue at position 170 and/or an Alanine residue at position 229 relative to SEQ ID NO: 1.

4. The recombinant Bordetella pertussis bacterium of claim 3, wherein the LpxA protein comprises (i) a Serine residue at position 170 and (ii) an Alanine residue at position 229 when numbered in accordance with SEQ ID NO:1.

5. The recombinant Bordetella pertussis bacterium of claim 4 which comprises an LpxA gene that encodes an LpxA protein having at least 80% amino acid sequence identity to SEQ ID NO: 1 or 2.

6. The recombinant Bordetella pertussis bacterium of claim 4 which comprises an LpxA gene that encodes an LpxA protein having the amino acid sequence of SEQ ID NO: 2.

7. The recombinant Bordetella pertussis bacterium of claim 1, wherein the heterologous LpxD gene encodes an LpxD protein having at least 90% amino acid sequence identity with SEQ ID NO:3 or SEQ ID NO: 4.

8. The recombinant Bordetella pertussis bacterium of claim 6, wherein the heterologous LpxD gene encodes an LpxD protein having at least 95% amino acid sequence identity with SEQ ID NO:3 or SEQ ID NO: 4.

9. The recombinant Bordetella pertussis bacterium of claim 7, wherein the heterologous LpxD gene encodes an LpxD protein having an amino acid sequence selected from the group consisting of SEQ ID NO:3 or SEQ ID NO: 4.

10. The recombinant Bordetella pertussis bacterium of claim 1, wherein the endogenous LpxA gene is inactivated and/or the endogenous LpxD gene is inactivated.

11. The recombinant Bordetella pertussis bacterium of claim 1 which produces lipid A wherein (i) at least 30% of the C3′ acyl chains are from about C10 to about C12 in length; and/or (ii) at least 30% of the C2′ acyl chains are from about C10 to about C12 in length; and/or (iii) at least 30% of the C2 acyl chains are from about C10 to about C12 in length.

12. The recombinant Bordetella pertussis bacterium of claim 1 that produces Lipid A with reduced endotoxic activity compared to that of the Lipid A produced by the parental strain, particularly when measured using TLR4 stimulation assays, particularly human TLR4 stimulation assays.

13. The recombinant Bordetella pertussis bacterium of claim 1, wherein the growth curve of a bacterial population of said bacteria is comparable to the growth curve of a population of the parental strain.

14. An isolated outer membrane vesicle (OMV) derived from the recombinant Bordetella pertussis bacterium of claim 1 which comprises modified Lipid A incorporated into the membrane wherein substantially all of C3′ acyl chains of the Lipid A have a length of C10 and/or wherein substantially all of C2 and C2′ acyl chains of the Lipid A have a length of C12.

15. The isolated outer membrane vesicle according to claim 14 which is substantially free of dermonecrotic toxin and/or which contains endogenous genetically detoxified pertussis toxoid.

16. An immunogenic composition comprising at least one isolated OMV according to claim 14 and a pharmaceutically acceptable excipient.

17. The immunogenic composition of claim 16, further comprising at least one additional antigen selected from the group consisting of (1) pertussis toxoid (PT), (2) FHA, (3) pertactin (PRN), (4) FIM2/FIM3, (5) adenylate cyclase, (6) diphtheria toxoid (DT), (7) tetanus toxoid (TT), (8) inactivated polio virus (IPV), (9) hepatitis B surface antigen and (10) Hib PRP.

18. A method of inducing an immune response in a suitable animal, which comprises administering to said animal the OMV of claim 14.

19. A method of vaccinating a suitable animal, which comprises administering to said animal a vaccine composition comprising the OMV of claim 14.

20. A method of modulating the reactogenicity of the Lipid A of a Bordetella pertussis bacterium, comprising stably integrating into the genome of the bacterium at least one gene selected from the group consisting of LpxA and LpxD, wherein:
(i) the LpxA gene encodes an LpxA protein having at least 80% sequence identity with SEQ ID NO: 1 or 2 and wherein the protein comprises Serine at position 170 (S170) and/or Alanine at position 229 (A229) when numbered in accordance with SEQ ID NO: 1 or 2; and/or
(ii) the LpxD gene encodes an LpxD protein having at least 90% amino acid sequence identity with SEQ ID NO:3 or SEQ ID NO: 4.


SEQUENCE LISTING (24 sequences)

SEQ ID NO: 1 - protein, 264 aa, Bordetella pertussis
MSGNIHPTAVVDPAAQIDSSVVIGPYSVVGPGVSIAAGTEVGAHCVLDGVTSIGRDNRFYRFCSIGGMPQDKKYSGEPTRLVIGDRNTVREFTTFNTGTVQDGGVTSIGDDNWIMAYVHIAHDCHIGNNTILANSVQLGGHVQVGDWAIVGGLTGVHQFAKIGAHSMTGGNSSLMQDAPPFVLAAGNPCRPVGVNVEGLKRRGFSAAAISALRDAYKSIYRRGLSLDEGRAELRARQQAEPDVAEHLQTMLDFLDASTRGIIRP

SEQ ID NO: 2 - protein, 264 aa, Bordetella parapertussis
MSGNIHPTAVVDPAAQIDSSVVIGPYSVVGPGVSIAAGTEVGAHCVLDGVTSIGRDNRFYRFCSIGGMPQDKKYSGEPTRLVIGDRNTVREFTTFNTGTVQDGGVTSIGDDNWIMAYVHIAHDCHIGNNTILANSVQLGGHVQVGDWAIVGGLTGVHQFAKIGAHSMTGSNSSLMQDAPPFVLAAGNPCRPVGVNVEGLKRRGFSAAAISALRDAYKSIYRRGLSLDEARAELRARQQAEPDVAEHLQTMLDFLDASTRGIIRP

SEQ ID NO: 3 - protein, 333 aa, Comamonas testosteroni
MSVLLGQILDALGGELVGGEREMPISRIAPLDSAGHGDLSFLSNPRYRQQLAASQAACVIVAPTMRDEAAQRGACIVTADPYAYFARATQWWKAHQQGGRPRGIHASAVVDATAQVHESAYVGPQCVVEAGAVIGADTVLKSRVTISQGCVLGERCILHPGVVIGADGFGFAPSAGQWEKIEQLGAVRIGNDVEIGANTCVDRGALDDTVIEDGVKIDNLVQIAHNVHIGAHTVIAGNTGIAGSARIGKRCQIGGAANILGHLTIADGTVISPTSMVTRSLPKAGFYTGIFPLQENEQWEKNAATFRQLYTLRERVKKLEQALAEGRQSNNGN

SEQ ID NO: 4 - protein, 353 aa, Pseudomonas aeruginosa
MMSTLSYTLGQLAAHVGAEVRGDADLPIQGLATLQEAGPAQLSFLANPQYRKYLPESRAGAVLLTAADADGFAGTALVVANPYLAYASLSHLFDRKPKAAAGIHPTAIVAADAEVDPSASVGAYAVIESGARIGAGVSIGAHCVIGARSVIGEGGWLAPRVTLYHDVTIGARVSIQSGAVIGGEGFGFANEKGVWQKIAQIGGVTIGDDVEIGANTTIDRGALSDTLIGNGVKLDNQIMIAHNVQIGDHTAMAACVGISGSAKIGRHCMLAGGVGLVGHIEICDNVFVTGMTMVTRSITEPGSYSSGTAMQPAAEWKKSAARIRQLDDMARRLQQLEKRLAAVTSSGDASSDA

SEQ ID NO: 5 - protein, 178 aa, Bordetella bronchiseptica
MQFLKKNKPLFGIVTLALACATAQAQPTQGGVSLHYGIGDHYQRVTLNYETPTLWSHQFGGNWGRLDLTPELGASYWWADGSRSPGHVWQASAIPMFRWWTGERFYIEAGIGATVFSSTSFADKRIGSAFQFGDHIGLGFLLTPSNRIGLRYSHFSNAGIKEPNPGLDIVQLTYTYQF

SEQ ID NO: 6 - protein, 239 aa, Francisella novicida
MLKQTLQTNFQGFKDIFKKPKLHNHKLPRYLQLKYTFIPLLILVIFAYYNLDTPVENYIKHSMPNIVGVIFGKITDVGKAEYILIICGVIVLARLFTDSQKLSANTRAMFDKVSAYAGFILATVAISGILGQILKMIIGRARPKFFLEYGSHYFQHFHAPGYDFASMPSGHSITVGAMFIAFFYIFPKLRYFWYLLIVVFAGSRIMVGSHYPSDVIFGVAFGCYCTAYIYYWMRNREII

SEQ ID NO: 7 - protein, 226 aa, Rhizobium leguminosarum
MPPLRWQACLLITLNAVILSMLLFDAPIGASEAPAPVKHLGEILTGFGDSAWLIYTSILLFFQGRAGYKLVKTARSKAQALYVSWIGAYLFITVVFSGLLANLLKRAIGRARPDHFHDYGMFSFTPFSGHAAFESFPSGHSTTVGAFFAAFALLFPRYRIAFIACAIWLGMTRVMVGAHYPSDVIAGLAFGAWFSLLTAIVFARCGLLFKLAPDGWPLSKRLFRTA

SEQ ID NO: 8 - protein, 222 aa, Francisella novicida
MARFHIILGLVVCFFAWIFFLIFPNLDIQFAGHFYNSSAHQFIGGYDGFLGFLHWFARFFPIFFSIIVILFLLGSLFIDKFKIKYRKAIFFIAVCLWIGPGLVVNYVFKDHWGRPRPVMVEQFNGDKIFQPPFVISSQCDKNCSFVCGDASMGFWLFAFMPLLATRKKKLVAFIAAVVAGGGLGLMRMSQGGHFFSDVVFCGIFVYISTWVVYALMYRKKEY

SEQ ID NO: 9 - DNA, 795 bp, Bordetella parapertussis
atgtcaggaaacatccatcccaccgccgtggtcgatccggcggcacagatcgacagttcggtcgtgatcggcccatacagcgtggtggggccgggagtcagcattgccgcgggcaccgaggtcggcgcgcactgcgtgctcgacggcgtgaccagcatcgggcgcgacaaccgcttctaccgcttttgctccatcggcggcatgccgcaggacaagaagtacagcggcgaaccgacgcgactggtcatcggcgaccgcaacacggtgcgcgagttcaccacgttcaacaccggcacggtgcaggacggcggcgtgaccagcatcggcgacgacaactggatcatggcctacgtgcacatcgcgcacgactgccatatcggcaacaacaccatcctggccaactcggtgcagctgggcggccacgtgcaggtgggcgactgggccatcgtgggcggcctgaccggcgtgcaccagttcgccaagatcggcgcgcacagcatgaccggcagcaacagctcgctgatgcaggatgcgcccccgttcgtgctggcggcgggcaatccgtgccggcccgtgggcgtcaatgtcgaaggcttgaagcggcgcggtttctcggcggcggccatctcggcgctgcgcgatgcgtacaagtcgatctaccggcgcggactgtcgctggacgaggcgcgcgccgagctgcgcgcgcgccagcaggccgagccggatgtcgccgagcatctgcagaccatgctggacttcctggacgcttcgacgcgcggcatcatccggccatga

SEQ ID NO: 10 - DNA, 1002 bp, Comamonas testosteroni
gtgagcgttctattgggacagattctcgatgcgctcggtggagaactggtaggtggcgagcgcgagatgcctatctcgcgcatcgctccactggactctgcgggccacggtgatctgagctttttaagcaatccccgctatcgccagcaactggccgcctcacaggcggcctgtgtcattgtggcgccgacgatgcgtgatgaagcagcgcagcgtggcgcctgcattgtcacggctgatccttatgcctatttcgcgcgagccacccagtggtggaaggcgcatcagcagggcggcaggccgcgcggcatccatgcgagtgcggtggtggatgccacggcccaggtgcatgaaagtgcctatgtggggccgcaatgcgtggtggaggcaggcgcggtcatcggtgccgatacggtgctgaaatcgcgcgtcaccatcagccagggctgtgtgctgggcgagcgctgcattctgcatcccggtgtggtcatcggtgccgacggcttcggcttcgcgccctcggcgggccaatgggaaaagatagagcagttgggggccgtgcgcatcggcaacgatgtggaaatcggtgccaacacctgcgtggatcgtggggcacttgatgacaccgtcattgaagatggcgtcaagatcgataatctggtgcagattgcccacaatgtgcacattggtgcgcatacggtgattgccggcaataccggtattgccggcagtgcgcgcatcggcaagcgctgccagattggtggagccgccaatattctggggcatttgacgattgcggatggcacggtgatttcgccgacctccatggtcactcgctccttgcccaaggcaggtttttatacaggcatctttccgttgcaagagaacgagcaatgggaaaagaacgctgcaaccttcaggcagttgtacacgctccgggagcgggtcaagaagcttgaacaagcgctggctgagggccggcaaagcaacaacggaaattga

SEQ ID NO: 11 - DNA, 1062 bp, Pseudomonas aeruginosa
atgatgagtaccttgtcctacaccctgggccagttggccgcgcatgtcggcgcggaagtccgtggcgacgcggacctgccgatccagggcctggcgaccttgcaggaggccggaccggctcagctgagcttcctggccaacccgcagtatcgcaagtacctgcccgaaagccgtgccggcgccgtcctgctgacggctgccgacgccgacgggttcgccggtaccgcgctggtggtggccaacccctacctggcctatgccagcctgtcgcacctgttcgatcgcaaaccgaaggcggccgccggtatccatcctaccgccatcgtcgcggcggacgccgaggtcgatccgagcgccagcgtcggcgcctatgcggtgatcgagagtggcgcccgcatcggcgctggcgtcagcatcggcgcgcactgcgtgatcggagcccgtagcgtgatcggcgaaggcggctggctggcgccgcgagtcacgctctaccacgacgtcaccatcggcgcgcgggtgagtatccagtcgggcgcggtgatcggcggcgaaggtttcggtttcgccaacgagaagggcgtctggcagaagatcgcccagatcggcggggtgaccatcggtgacgacgtcgagatcggcgcgaacaccaccatcgaccgtggagcactgtccgatacgctgatcggcaacggtgtgaagctggacaaccagatcatgatcgcgcacaacgtgcagatcggcgatcataccgccatggccgcttgcgtcggcatttccggcagcgccaagatcggcaggcattgcatgctcgccggcggcgttggcctggtcggccatatcgagatctgcgacaacgttttcgtgaccggcatgaccatggtgacccgctcgatcaccgagccgggatcgtattcctccggtaccgccatgcaaccggcggccgaatggaaaaaaagcgccgcgcgcattcgtcaactggacgacatggcgcgtcgacttcagcagctggaaaaacgcctggctgccgtgacctcaagcggtgacgcttcatctgatgcgtaa

SEQ ID NO: 12 - DNA, 1002 bp, Artificial Sequence
  LpxD Comamonas testosterone (codon optimised)
atgagcgtgctgctgggccagatcctcgatgcgctgggcggcgaactggtgggcggcgagcgcgaaatgcccatcagccgcatcgcgccgctggatagcgcgggccatggcgatctgtcgtttttaagcaacccccgctaccgccaacaactggccgccagccaagccgcttgtgtgattgtcgccccgaccatgcgcgacgaagccgcccagcgcggcgcttgtatcgtgaccgccgatccgtacgcctatttcgcccgcgccacccagtggtggaaagcccaccagcaaggcggccgcccgcgcggcatccacgccagcgccgtggtcgatgccaccgcccaagttcacgagagcgcgtatgtcggcccccagtgcgtcgtcgaggccggcgccgtgatcggcgcggacaccgtgctgaagagccgcgtcaccatcagccaaggttgcgttttaggcgagcggtgtattttacatccgggcgtggtgatcggggccgatggcttcggcttcgcccccagcgctggccaatgggaaaagatcgagcagctgggcgccgtgcgcatcgggaatgacgtggagatcggcgccaacacgtgcgtggaccgcggcgcgctggacgacacggtcatcgaggacggcgtgaagatcgacaatttagtgcagatcgcccacaacgtccacatcggcgcgcacaccgtcatcgccggcaacaccggcattgccggctcggcccgcattggcaagcgctgccagattggcggggccgccaacattttaggccatttaaccatcgccgatgggaccgtcatcagcccgacctcgatggtcacccgctcgctgcccaaggcgggcttctacaccggcatcttcccgctccaagaaaacgagcagtgggagaagaatgcggccaccttccgccagctgtacactttacgcgagcgcgtgaagaaactggagcaagctctggccgaggggcgccagagcaacaacggcaactga

SEQ ID NO: 13 - DNA, 1062 bp, Artificial Sequence
  LpxD Pseudomonas aeruginosa (codon optimised)
atgatgagcactttaagctacactttaggccagctggccgcccatgtgggcgccgaggtccgcggcgacgccgatctgcccattcaaggtttagccacgctgcaagaagccggcccggcgcagctgagctttttagcgaacccccagtaccgcaagtacctcccggagtcgcgcgccggcgccgtgctgctgaccgccgcggacgccgacggcttcgccggcaccgctttagtcgtggccaatccctatttagcgtacgcctctttatcgcacctcttcgatcgcaaaccgaaggccgccgcgggcatccacccgaccgcgatcgtggccgccgatgccgaagtggacccgagcgcgagcgtgggggcctatgccgtcatcgagagcggcgcccgcatcggcgccggcgtgagcatcggcgcgcattgcgtgattggcgcgcgcagcgtcattggcgaaggcggctggctggcgccgcgcgtgactttataccatgacgtgaccatcggcgctcgcgtcagcattcagagcggcgccgtcatcgggggcgagggctttggcttcgccaacgagaagggcgtctggcagaaaatcgcccaaatcggcggcgtcaccatcggcgacgacgtggagattggcgccaacaccaccatcgatcgcggcgcgctgtcggacaccctcatcggcaatggcgtcaagctggacaaccagattatgatcgcgcacaacgtgcagatcggcgaccataccgccatggccgcttgtgtgggcattagcggcagcgccaagatcggccgccactgcatgctggccggcggcgtgggtttagtgggccatatcgagatttgcgacaacgtgttcgtcaccggcatgaccatggtgacccggagcatcacggagccgggctcgtacagcagcggcaccgcgatgcaaccggccgccgagtggaagaagtcggccgcccgcatccgccaactggatgacatggcgcgccgtttacaacagctggagaagcgcctcgccgccgtgaccagcagcggcgatgccagcagcgacgcctga

SEQ ID NO: 14 - DNA, 93 bp, Artificial Sequence
  N-term insert for genomic integration into B. pertussis
accatctgcaagtaccagggccgggccctggtcgacggccaactggtcgccgaagccaagctgatgtgcgcgatccgtagcctggaagagtaa

SEQ ID NO: 15 - DNA, 491 bp, Artificial Sequence
  C-term insert for genomic integration into B. pertussis
gcctgcgcataggcatggtcgccggcgagccttcgggcgacctgctggccgggcgcatcatcgccggcctgcaggcccgggcacccggcgtgcactgcgccggcataggcgggccccagatggcggcgcgcggcttcgaggcctggcaccccatgcatgcgctcacggtgttcggctacatcgatgcgttcaagcggattcccagcctgctgtcgacctacggcgacgtcaagcggcgcctgctggccgagccgccgtcggtgttcgtgggcatcgacgcgccggatttcaacctgcgcctggagcaccagttgcgccaggccggtacccccaccgtgcatttcgtcggtccctcgatctgggcctggcgctacgaacgcatcaacaagatccgcgccgccgtctcccacatgctggtgctgtttccgttcgaggaagcgctctatcgcaaggaaggaatcccggtcacctatgtcggccatccgctggcc

SEQ ID NO: 16 - DNA, 537 bp, Bordetella bronchiseptica
atgcaatttctcaagaaaaacaagcccctgttcggcatcgttacactggctctggcatgtgccaccgcccaggcgcagcccactcagggcggggtcagcctgcattacggtattggcgaccactatcagcgcgtcacgctgaactacgaaacccccacgctctggagccaccagttcggcggcaattggggccgcctggacctgacccccgaactgggcgcgtcatactggtgggccgacggctcgcgctcgcccggccacgtgtggcaggccagcgccattccgatgttccgctggtggaccggcgagcgcttttacatcgaggccggcatcggcgccacggttttcagcagcaccagcttcgccgacaagcgcatcggttcggccttccagtttggcgaccatatcgggctgggcttcctgctgacgcccagcaaccgcatcggcctgcgctattcgcacttctccaacgccggcatcaaggaaccgaaccccggcctcgatatcgtgcagctgacctatacgtaccagttctga

SEQ ID NO: 17 - DNA, 296 bp, Artificial Sequence
  Porin promoter (BP0840 promoter; BX640413.1174494-174789)
tccttgagtgaactggggggtcggcgatttccagtttctcaaatccggttcggatgaaccatgcatacaacctattgaatcttcacagttagcccgcgcgcgattccggattaaggggacaggatgttgcaacttaccaacaatggggcgggaaaccggcttttttctgagcctgaccatagccagtcctgccgatttttgatgcaatagcgtcaacttcccttcgcggagttagggggggcggcaggccggtatgtgctgcattgctgctcttgtcactcaaatcaacggagatt

SEQ ID NO: 18 - DNA, 1620 bp, Bordetella pertussis
atgactctcgctacccgatccatgcaaccccatgcggtttcccctggccaggccaggtcctggcccctgcccgcggcgggctggctgcttctcgcggtcggcgtctggctggccttcctgtcctggatgcggcccctggcgctgcccgacgaagggcgctacgccggcgtggcctgggacatgctgcgcaacggctcgttcgcggtgccgctcatcgacggcatgccgtatttccacaagccgccgctgtactactggctggccgagctgtcgttccgcctgttcggcgtcaacgaatgggcggcgcgcctgccctcggcgctggccgcctgggccagcgccgtcgcgctgtacctgttcgtgcgccgccatcgcgacgccgcgtccgcgacgctctgcgtgctggtgctggccacgctgccgctgtttttcggcggcgcccagtacgccaacatggatatgctggtggccggcatgatcacgctgtgcgtgctggccggcgccgacacggcgctgcgtgtccgcggcgggcaggcctggcgcgccatggcgctggccaccggcgtgtgcgccgcgctggccatgctggccaaggggctgatcgggctggtgctgccgggcgcgatcctgctggcctggctggcgtggcggcgcgactggcgcggcctgcgggccttgctgtggccgcccgccatcctggcgttcgcggtggtggccgtgccctggttctggctgatgcaggtacgctacccgggctttttccagtacttcttcgtccaccagcatttcgagcgtttcgcgcagaccggcttcaacaatgtgcagccgttctggttctacctgcccgtcatcgcgggcctggcattgccgtggtcgttgtgggccggcggcttgctgcgcaagcaattctgggccgccgacgccgacccggacggcttgcgccgcctggcgctggtctggctggcggtcatcgtggcgtttttctccatgccgcagtccaagctggtcggctacatcatgccggtgctgccgccgctggccttcctgctggccgaagtggtgatgggcgccttgcgcgacccggcggtggcgcgggccacgcgccgcatggcgcgcgtcagcgccctggtggcggtggccatctgcgtgaccgcggtgttcgtggcgtcgttcaacgcccgcggcagttcgcgcgagctggccctgtcgctgcgcggcgagctgcggccggacgacacgctggtggccctgcatacctatccgttcgacctgcagctgtatgcccatgcggcccggccgatgtgggtggtcgacgactggagcaaccccgaaattcccaagcgcgacaattggcgcagggagctctacgacgcggtccagttcgagccggccctgggcgagcggctgctggtctcggcagatactttccagcagcgcctgtgccaggcgccggagggcagccgttactgggtctggggcacggcggccgacgaagaggcgtacgcgccgctgcgcggccaggcggcgcgctttgccgacgcgcggcgcagcctctggctggtgctggtggacgaagccttcaaggggcgtgtctgtgacggaacgcccacaggcggctag

SEQ ID NO: 19 - DNA, 4395 bp, Bordetella pertussis
gtggataaagatgaatcggcattgcggcaacttgtcgacatggcgcttgtaggctacgacggcgtggtggaagagttgctggcgctgccctcggaagagtccggggatcttgcgggtggacgggccaaacgagagaaggccgaattcgcgctgtttagcgaagcgcccaacggggatgaacccatcggccaggatgctcgtacatggttctattttcccaagtaccgcccggtggcggtttccaatttgaagaaaatgcaggtggcgattcgtgctcgtctcgagcctgagtcattgatcctgcagtggttgatcgcactcgatgtctatctcggcgtgttgatcgccgccttgtcccgcactgtgatcagcgacctggtattcgagtatgtcaaggcgcgctacgagatctactacctactgaatcgcgtgccgcatccgctggcgacggcgtacctcaagcgccgccgccagcgtccggtggatcgttcgggacgtctgggctcggtgttcgaacacccgctatggttcgcctacgacgaattggccggcaccgtcgatctggatgccgacatctacgagcaggcgctcgccgaaagcatcgaacgccgcatggacggagagccggacgacggcagtctggataccgccgaacacgacgtatggcgtttgtgccgcgacggcatcaatcggggcgagcaggcgattttccaggcatccggcccatatggcgtggtcgcggatgcagggtatatgcgcaccgttgccgatctggcgtatgccgatgcattggcggattgcctgcatgcccaactgcgcatccgggcgcaaggctcggtcgatagcccgggagacgagatgccacgcaaacttgatgcgtgggaaatcgccaagtttcatctggccgcgacccagcaggcgcgggttgatctgctcgaggcggcgttcgcgctcgactacgccgccttgcgcgatgtgcgcgtctacggcgattaccgcaatgcgctggctcttcggttcatcaagcgcgaggccttgcggttgctgggggcgcggcgcggcaacgcctctacgatgccggcggttgcggctggcgagtacgacgaaatcgtggccagtggagcagccaatgacgcggcttatgtatccatggccgccgcgttgatcgcgggcgtgctctgcgatctcgagagcgcgcagcgcacgttgcccgtcgtattggccaggtttcggccccttggcgtgcttgcgcgattcagaaggctggagcaggaaaccgcgggcatgctgcttggcgaccaggagccggagcctcggggcttcatcagttttaccgattttcgcgatagcgacgcgttcgccagctacgcggagtatgcggcccagttcaacgactatatcgatcaatacagcatactcgaggcgcagcggctggcgcggattctggccctgggctcgcggatgacggtcgatcaatggtgccttcccctgcagaaagtacggcactacaaggtgctgacatcgcagccagggctgatcgcgcgtggaatcgaaaatcacaacaggggcattgaatattgcctggggcggccgccgctgaccgatctgccgggtcttttcaccatgttccagctccatgattccagctggctgttggtatcgaacatcaacggtgagctttggtctgatgtccttgcgaacgctgaggtgatgcagaatcctacgctggccgcccttgccgagccgcaaggccgctttcgaaccggccgtcgaacgggcgggtggtttctcggcggccccgcgactgaagggccaagtctgcgcgacaactacctcctcaaattgaggcagagcaatccggggctggatgtcaagaaatgctggtatttcgggtatcggcaagagtacaggctgcccgcaggcgcgctgggtgtgccgctattcgctgtttccgttgccctgaggcacagtctggacgacctcgcggcgcacgcgaagtccgctttgtacaaacccagcgaatggcagaagttcgccttctggatcgtgccgttctaccgggaaatattcttttcgacccaggatcgttcctatcgggtcgatgtggggagcatcgtctttgattcgatttccttgcttgcctcggtgttcagcataggagggaagttgggcagtttcacccgtacccagtatggcaacctgcgcaatttcgtggtgcggcaacgcatcgcggggttgagcgggcagcgcttgtggcgttcggtgctcaaggagcttccggcattgatcggagccagcgggttgcgcctgtcgcgttcgctgctcgtcgatctgtatgagatcttcgagcccgtgcctatccgtcggcttgtcgcgggattcgtgagcgccactacggtcggcgggcgtaaccaggccttcctgcggcaggcattctccgctgccagttcctcggccgggcgcacggggggccaattggcaagcgaatggcggatggccggcgtggacgccaccggtttggtcgagtccacgtcgggcggcaggtttgagggcatctacacgcgcggcttgggaccgttgagcgagtgcaccgagcacttcatcgtcgaatccgggaatgcctacagagtcatctgggatgcatacacgcatggttggcgcgtggtcaatgggcgtttgccgccgaggctgacctataccgttccggttcggctgaacgggcaaggccactgggagacgcatctggatgtccctggccggggtggggcgccggaaatcttcggacgcatccgcacgcgtaatctggtcgcccttgccgccgagcaggccgcgccgatgcgccgcctgctcaaccaggcgaggcgtgtggcgctcaggcatatcgatacctgcaggagcaggcttgcgttgccgcgcgctgaatccgatatggacgcggcgatccggattttcttcggagagccggacgccggccttcgccagcgcatcgggcgacgcctgcaggaggtcagggcctatatcggcgatctgagtccggtcaatgacgtgctgtaccgggcgggatatgacctcgacgatgtcgcaacgctgtttaacgcagtggaccggaacacgtcgctgggcaggcaggctcggatggagttgtatctggatgccattgtcgatttgcatgccaggctcggctatgaaaatgcgcgttttgtcgacctgatggcgttccacctgctcagcctgggccatgccgcgacggccagtgaggtcgtggaggccgtttcgccccggctgctgggcaatgtgttcgatatctcgaacgtcgcccagctcgaacgcggtatcgggaatcccgccagcaccggtcttttcgtcatgctgggcgcctattcggaatcgtcgcccgcgatattccagtcgttcgtgaacgatatatttcccgcatggcgacaggcttccggcggggggccgctggtatggaacttcggtcctgccgccatcagcccgacgcgcctggattacgcaaataccgatatcggattgctcaaccatggggatatttccccgctgcgcgccaggccgccattgggcggcaggcgcgacatcgatcttcctccggggctggatatttcgttcgtgcgttacgaccgcccggtgcgcatgtccgcgccgcgtgcgctcgacgccagcgtcttcaggccggtcgacgggcctgtccatggctatattcaatcgtggacgggggctgagatcgaatatgcgtacggcgcgcccgcagcggcacgcgaggtcatgctgaccgacaatgtgcggatcatcagcatcgagaacggcgatgaaggggcgatcggggtgcgcgtgaggctcgacactgttcccgtcgcgacgccgctcatcctgactggcggctccttgagcgggtgcacgacgatggttggggtcaaggagggctacctggccttctaccacactggcaagtcgaccgaactcggggattgggcgactgcgcgcgaaggcgtacaggcgctttaccaggcccatttggcgatggggtacgcgccgatctcaatcccggcgccgatgcgcaatgacgacttggtcagcatcgccgcgacctacgaccgggcggtcattgcctatctgggcaaggatgtgccgggaggcggcagtacgcgcataacgcgtcacgatgagggggcgggcagtgtggtttcgttcgactacaacgcagcagtccaggcgtcggccgttccccgcctcggccaggtatacgttctgatttccaacgacggacaaggggcgcgagccgtcctgctggcggaggatctggcctgggcaggcagcggcagtgccttggatgtgttgaacgaacgattggtgacgttgtttccggcgccggtctga

SEQ ID NO: 20 - DNA, 1427 bp, Artificial Sequence
  Construct for ArnT KO
gtgtgccagctgggaaatcatcgtcgtcgacgacggcagcaccgacgataccgccgagctgatggcgcaatggtccgcggtcgagggcatccgctacgtgcagctgtcgcgcaatttcggcaaggaggccgccctgacggccggcctggaagccgcggacggcgacgccgtcatctgtctggacgccgacatgcagcatccgcccgaactgataggcgacatgctggcggcctggcgcaacggcgccgacatggtctacgcggtgcggcgccagcgcgacgacgagccgtggttcaagcgtgtcggcgcccgcgcgttctaccgcctgctgagcacggcgcgcggcgtcgaggtgccgccgcacgcgggcgacttccgcctgatggaccgccgcgtggtcgaggccctggtggcgctgcccgaacgcacccgcttcatgaaggggctgtatgcctgggtgggcttcaagtcgcaggccgtgccctacaccccgcaagcgcgccgccatggcgccagccacttcagcgcctggaagctgtttcgcctggcctgcgacggcctgaccgccttcaccacctggccgctgcggctggtcagcctcatcggcgtgctgttcgcgctgctgtcgctgtcctacggcggttacctggtggccgactatctcatcagcggcaacgcggtgtccggctggaccaccatcgtcaccgcgctgctgttctttgccggcatcaacctgatttcgctgggcgtggtgggcgagtacgtggcccggatcttcgacgaagtgaaaggccggcccctgttcatcgcccgtcaacgccgcgggcgcgccaagcgcgcggccaaggcgcgttcccaatgactctcgctacccgatccatgcaaccccatgcggtttcccctggccaggccaggtcctggcccctgcccgcggcgggctggctgcttctcgcggtcggatagctcgagtgacggaacgcccacaggcggctagcgacgaatgtcaggaaggccagcccgatcaacaccagggccagcagcacgtcatagggcagcgacgtggcgtgcagcagccaggcatatgacaactcattgatggcgaaacccagggctgacacaaggaagaaacgccgggcggctatggtccaggatgccgcaaggtggcggaaagtgagtctgaaatgaccagtgaacgatacgacgaaggccaacagccagcccaccacgttggcccccagcgggggcaggccggcccattccacgcaggcgaccgcgacggcccaatgggtggcggccgcggcgcatccgacggcaatgaaccaggcaatctgtcgcagcaggccgcgcatgtcgaagaggtgtctcccttgaggaaccaggccggcgatgtccgttgccggcgcatcgcggtgtgcggcgatgatttt

SEQ ID NO: 21 - DNA, 2705 bp, Artificial Sequence
  Construct for replacing DNT with LpxD P. aeruginosa
cgccgccagcatttccgaggcgctcggctacgagcccggcgtcaacggtatccacgcgccagcggctgcctgagtccggcctggcctggcgataaggcggtgcgtgcaggcgtggctgcgatcgcaccgtaaccgttggtgcggcgtcctgatctccggagcgttcggctgggagatggctgcgcatccgatgggccggttcttgtgtgtttgctagcctgactaacaggcgacctggggtcgtttccggtttccggtcaggggcaaacaaacgtggataaagatgaatcggcattgcggcaacttgtcgacatggcgcttgtaggctacgacggcgtggtggaagagttgctggcgctgccctcggaagagtccggggatcttgcgggtggacgggccaaacgagagaaggccgagttcgcgctgtttagcgaagcgcccaacggggatgaacccatcggccaggactgagagctctccttgagtgaactggggggtcggcgatttccagtttctcaaatccggttcggatgaaccatgcatacaacctattgaatcttcacagttagcccgcgcgcgattccggattaaggggacaggatgttgcaacttaccaacaatggggcgggaaaccggcttttttctgagcctgaccatagccagtcctgccgatttttgatgcaatagcgtcaacttcccttcgcggagttagggggggcggcaggccggtatgtgctgcattgctgctcttgtcactcaaatcaacggagattggtaccatgatgagcactttaagctacactttaggccagctggccgcccatgtgggcgccgaggtccgcggcgacgccgatctgcccattcaaggtttagccacgctgcaagaagccggcccggcgcagctgagctttttagcgaacccccagtaccgcaagtacctcccggagtcgcgcgccggcgccgtgctgctgaccgccgcggacgccgacggcttcgccggcaccgctttagtcgtggccaatccctatttagcgtacgcctctttatcgcacctcttcgatcgcaaaccgaaggccgccgcgggcatccacccgaccgcgatcgtggccgccgatgccgaagtggacccgagcgcgagcgtgggggcctatgccgtcatcgagagcggcgcccgcatcggcgccggcgtgagcatcggcgcgcattgcgtgattggcgcgcgcagcgtcattggcgaaggcggctggctggcgccgcgcgtgactttataccatgacgtgaccatcggcgctcgcgtcagcattcagagcggcgccgtcatcgggggcgagggctttggcttcgccaacgagaagggcgtctggcagaaaatcgcccaaatcggcggcgtcaccatcggcgacgacgtggagattggcgccaacaccaccatcgatcgcggcgcgctgtcggacaccctcatcggcaatggcgtcaagctggacaaccagattatgatcgcgcacaacgtgcagatcggcgaccataccgccatggccgcttgtgtgggcattagcggcagcgccaagatcggccgccactgcatgctggccggcggcgtgggtttagtgggccatatcgagatttgcgacaacgtgttcgtcaccggcatgaccatggtgacccggagcatcacggagccgggctcgtacagcagcggcaccgcgatgcaaccggccgccgagtggaagaagtcggccgcccgcatccgccaactggatgacatggcgcgccgtttacaacagctggagaagcgcctcgccgccgtgaccagcagcggcgatgccagcagcgacgcctgaaagcttttgaacgaacgattggtgacgttgtttccggcgccggtctgaatcgcattccgcgaaacggcgggcgcgcaggcctcgcccgatcggccggccgggccgcgcgatgcgcggcccggccgcattgggacgcggcctttattgcttttccacgcccgccttcttgaacagctcggcccattgcggcacctgctgcttgaccttggcttgcagcgcctcgggattagcctcgttggtcagcacctcggcgcccagctgcttcatgcgctcctggaacttgggatcggccaggccggcctgcaggctcttgaccagcttgtcgaccaccggcttgggcgtgcccttgggggcccacatgccgtgccagatacccacttcgaaacccttgtagccggactcgtccatggtgggcaggtcgggcagggtgggcacgcgcttgaggctggtgaccgcgtaggccttcaccttgccgctggtgatctgctgcgtggtattggtggtctggtcgcacatcaggtcgacctgcttgcccagcagatcgttcatggccggcgccgtgcccttgtagggaatggtcagcaggttgacgcccagcgcctcgaccagcatggtgccgcacaagtggctggccgcgccgatgccggcattggccagtgaaatcttgtcggcgttcttcttgacgtactcggccagttccttgatgttgttgggcgggaaatcgccgcgggcaatgatggtcatcggcacgtccaccaccaggccgatgggctcgaagctggtgtagggctcgtagccggggttcttgtacagcgagggcgcggtcgagaagccggcgtgcatgagcaggatcgagtagccgtc

SEQ ID NO: 22 - DNA, 720 bp, Artificial
  LpxE F. novicida (optimized for expression in B. pertussis)
atgctgaagcagaccctccagacgaacttccaaggtttcaaggacatcttcaagaagccgaagctgcacaaccacaagctgccccgctacctccagctgaagtacaccttcatccctttactgattttagtgatcttcgcgtactacaacctcgatacccccgtcgagaactacatcaagcacagcatgccgaacatcgtcggcgtcatctttggcaagatcacggacgtcggcaaggccgagtacattttaatcatctgcggcgtgatcgttttagcccgtttattcaccgacagccagaagctgagcgcgaacacgcgcgcgatgttcgacaaggtctcggcctatgccggcttcattttagccacggtggccatctcgggcattctgggccagattttaaagatgatcatcgggcgcgcgcgccccaagttctttttagagtatggcagccactacttccaacacttccacgcccccggctacgactttgccagcatgcccagcggccactcgatcacggtcggcgccatgttcatcgccttcttctacatcttcccgaagctgcgctatttctggtatttactcatcgtcgtgttcgccggctcgcgcatcatggtcggctctcactatccgagcgacgtcatcttcggcgtcgcgttcggctgctactgcaccgcctatatctactactggatgcgcaatcgcgagatcatctaa

SEQ ID NO: 23 - DNA, 735 bp, Artificial
  LpxE R. leguminosarum (optimized for expression in B. pertussis)
atgcgcgccttctgggcctctttagaccgccgctggcgccgcagcggcgtgggcatgccgccgctgcgctggcaagcatgtttatttgtcactttaaacgcggtgattttaagcatgctgctgttcgacgcgccgatcggcgcgagcaaggcccccgccccggtgaagcatctgggggaactgctgacgggcttcggcgactcggcttggctgatctacaccagcatcttattattcttccaaggtcgcgccggctacaagctgctgaaaacggcccgctcgaaggcccaagccttatatgtcagctggatcggcgcctatctgttcttcaccgtggtcttcagcggtttactggccaatctgctgaagcgcgccatcggccgcgcccgcccggaccatttccacgactacggcatgttcagcttcgccccgttctcgggccacagcgccttcgagagcttcccgagcggccatagcaccaccgtgggcgcctttttcgccgcctttgctttactgttcccgcgctaccgcgtggcgttcatcgcttgtgccatctggctgggcatgacccgcgtgatggtgggcgcccattacccgagcgatgtcatcgccggtttagcctttggcgcttggttctctttactgaccgccatcgtgttcgcgcgctgtggtttactgttcaagctggccccggacggctggccgctcgcgaagcgtttattccgcaccgcgtaa

SEQ ID NO: 24 - DNA, 669 bp, Artificial
  LpxF F. novicida (optimized for expression in B. pertussis)
atggcccgcttccacatcattttagggctggtggtgtgcttcttcgcttggatcttctttttaatcttcccgaatttagacatccagtttgccggccacttctacaactcgagcgcccatcagttcatcggcggctacgacgggtttctgggctttttacactggttcgcgcgcttcttccctatcttcttcagcatcatcgtgattttatttttactgggctctttattcattgacaagttcaagatcaagtaccggaaggcgatctttttcatcgccgtgtgtttatggatcggccccggtttagtcgtcaactacgtcttcaaggaccactggggccgcccgcgcccggtcatggtcgagcagttcaacggcgacaagatcttccagccgcccttcgtgatctcgagccagtgcgacaagaactgcagcttcgtctgcggcgacgccagcatgggcttctggctgttcgcgttcatgccgctgctcgccacccgcaagaagaagctggtggccttcatcgccgccgtggtcgccggcggcggtttaggcctcatgcgcatgagccaaggcggccacttctttagcgacgtcgtgttttgcggcatcttcgtgtacatcagcacttgggtggtctacgccctcatgtaccgcaagaaggaatactaa